(19) United States
(12) Patent Application Publication
(10) Pub. No.: US 2009/0124566 A1
(43) Pub. Date: May 14, 2009

(54) METHODS AND COMPOSITIONS FOR THE TREATMENT OF ERYTHROCYTE DISEASES

(75) Inventors: Jen-Tsan Chi, Chapel Hill, NC (US); Gregory Lamonte, Durham, NC (US); Shao-Yin Chen, Taipei City (TW)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: DUKE UNIVERSITY, Durham, NC (US)

(21) Appl. No.: 12/134,574

(22) Filed: Jun. 6, 2008

Related U.S. Application Data
(60) Provisional application No. 60/942,508, filed on Jun. 7, 2007.

(51) Int. Cl. A61K 31/7088; C12Q 1/68

(52) U.S. Cl. 514/44; 436/6

(57) ABSTRACT

Methods to determine the susceptibility of a subject to erythrocyte diseases are provided. The methods comprise determining the miRNA compositions of erythrocytes from the subject. The present invention has discovered that erythrocytes comprise microRNA (miRNA) populations and the populations can be profiled or analyzed and used to determine the susceptibility for disease. The miRNA compositions can also be used to determine the severity of erythrocyte disease, and the features and clinical phenotypes of the erythrocyte disorders. Also provided are pharmaceutical compositions comprising erythrocyte miRNAs and methods for the treatment of a subject with an erythrocyte disease. In other embodiments of the invention, the miRNAs can be used to increase the life-span of erythrocytes through the introduction of an erythrocyte miRNA.

### CROSS-REFERENCE TO RELATED APPLICATIONS

This application claims the benefit of U.S. Provisional Application No. 60/942,508, filed Jun. 7, 2007, herein incorporated by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named 344774SEQLIST.txt, created on Jun. 6, 2008, and having a size of 4.5 kilobytes and is filed concurrently with the specification. The sequence listing contained in this ASCII formatted document is part of the specification and is herein incorporated by reference in its entirety.

### FIELD OF THE INVENTION

The invention relates to the involvement of microRNAs in erythrocyte diseases.

### BACKGROUND OF THE INVENTION

Human red blood cell (erythrocyte) diseases, including anemia and malaria, present huge economic and social challenges to the worldwide human race. Our ability to confront these challenges is hindered by our limited understanding of the diseased phenotypes of erythrocytes. For example, even though sickle cell disease (SCD) is an anemia disorder whose mutation was established in 1949 (Pauling et al. (1949) Science 110: 543-548), current characterization of SCD erythrocytes still cannot fully explain the molecular basis for their enormous clinical heterogeneity and abnormal interaction with malaria parasites. This gap in our understanding highlights a need for greater understanding of all the genetic components of erythrocytes and a conceptual framework for capturing and analyzing all genetic information on a genomic scale. Although recent advances in microarray technology and analytic tools have led to an explosion of knowledge in many human diseases, the application of these tools to erythrocyte diseases has been limited by the long-held belief that mature erythrocytes lack most RNA expression.

### BRIEF SUMMARY OF THE INVENTION

Methods to determine the susceptibility of a subject to erythrocyte diseases are provided. The methods comprise determining the miRNA compositions of erythrocytes from the subject. The present invention has discovered that erythrocytes comprise microRNA (miRNA) populations and the populations can be profiled or analyzed and used to determine the susceptibility for disease. The miRNA compositions can also be used to determine the severity of erythrocyte disease, and the features and clinical phenotypes of the erythrocyte disorders. Additionally, the miRNA composition can be used to predict the stage of the erythrocyte disease and to monitor disease progression and treatment. Also provided are pharmaceutical compositions comprising erythrocyte miRNAs and methods for the treatment of a subject with an erythrocyte disease by administering erythrocyte miRNA sequences or compounds that inhibit the activity of erythrocyte miRNAs. Further provided are kits comprising reagents sufficient for the detection of particular erythrocyte miRNAs.

In other embodiments of the invention, the miRNAs can be used to increase the life-span of erythrocytes. In this manner, an erythrocyte miRNA is introduced into an erythrocyte. Introduction of the miRNA leads to an increase in survival.

In yet other embodiments of the invention, erythrocyte miRNAs that can predict the susceptibility to or severity of an erythrocyte disease, that is indicative of the presence of an erythrocyte disease, or that can distinguish subtypes of an erythrocyte disease are identified using methods comprising obtaining erythrocytes from a population of subjects, wherein said population comprises subjects having the erythrocyte disease, determining the composition of miRNA present in said erythrocytes from each subject within the population, and performing an analysis of the miRNA composition from each subject within the population.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 demonstrates that mature human erythrocytes contain abundant miRNA. FIG. 1A illustrates the purification scheme used to obtain mature erythrocytes and reticulocytes from human blood. FIG. 1B presents the size distribution of RNAs of three erythrocyte samples and one peripheral blood mononuclear cell (PBMC) sample were assessed with Agilent Bioanalyzer with indicated ribosomal RNA bands. FIG. 1C (left) shows the miRNA expression pattern of three mature erythrocyte samples compared to that of two samples of the erythroleukemia K562 cell line and (right) the expression of erythrocyte-specific miRNAs in the CD34+ erythroid progenitor cells at indicated stages of erythroid differentiation in a previously published study (Lu et al. (2005) Nature 435: 834). FIG. 1D shows a Northern blot showing the expression of U6 and indicated miRNAs in K562 cells and erythrocytes, with the migration position of U6 and mature miRNAs indicated.

FIG. 2 shows that erythrocyte miRNAs show differentiation- and disease-specific expression. FIG. 2A demonstrates that the miRNA expression of whole blood was similar to erythrocytes and grouped together in one branch away from leukocytes with unsupervised hierarchical clustering. FIG. 2B provides the miRNA expression pattern assessed with TaqMan real-time assay used to identify 83 reticulocyte-specific (pink) miRNAs. FIG. 2C shows that the erythrocytes obtained from normal control (red) vs. sickle cell disease (dark red) have distinct miRNA expression patterns with several differentially expressed miRNAs indicated. FIG. 2D demonstrates that the average expression value of the 83 reticulocyte-specific miRNAs in the SCD erythrocytes is significantly higher than that of normal erythrocytes.

FIG. 3A shows the separation of all HbSS samples into two indicated subtypes based on the unsupervised analysis of global miRNA expression. FIG. 3B presents the reticulocyte percentage of type I vs. II HbSS patients. FIG. 3C demonstrates that the expression of miR-221, -23b is associated with high vs. low HbF expression. FIG. 3D shows the linear relationship between miR-221 and HbF expression levels.

FIG. 4 demonstrates that miR-320 is essential for the terminal differentiation of normal reticulocytes. FIG. 4A shows that SCD reticulocytes exhibit defective terminal differentiation. Purified normal (circle) and sickle (square) reticulocytes were placed in differentiation culture media containing autologous plasma. The percentage of remaining CD71+ cells after 24 and 48 hours was significantly higher for SCD samples than normal samples. FIG. 4B provides the target sequence (bolded) of the CD71 3′UTR of the five indicated species (SEQ ID NO: 1-5) aligned with the seed sequence of miR-320 (SEQ ID NO: 6). FIGS. 4C and 4D provide the relative expression of miR-320 among the normal vs. sickle erythrocytes. FIGS. 4E and 4F present the percentage of remaining CD71+ cells after in vitro differentiation at indicated time points after the transfection with either scrambled LNA or miR-320 knock down LNA. FIG. 4G presents the cell numbers of reticulocytes and erythrocytes 24 hours after transfection with indicated treatments.

FIG. 5 shows the accumulation of small-sized RNA during the intraerythrocytic life cycle of P. falciparum. FIG. 5A illustrates the four time points and the corresponding stages of P. falciparum. FIG. 5B presents the Bioanalyzer analysis of parasite RNAs at indicated time points. FIG. 5C presents the normalized intensity of small-sized RNAs at different time points.

FIG. 6 presents an analysis of human erythrocyte miRNA in P. falciparum. The expression of indicated genes in the samples is shown with DNA microarrays (FIG. 6A), Northern blots (FIG. 6B) and RT-PCR (FIG. 6C). FIG. 6D shows the assessment of the translocation potential of transfected biotinylated miR-181a (left) and miR-451 (right) with immunohistochemistry with microRNA (green), parasite membrane (red) and parasite nuclei (blue). FIG. 6E presents the expression level of miR-451 in the normal (red) vs. SCD erythrocyte (brown). FIG. 6F shows the alleviation of malaria resistance in HbSS erythrocyte by blockage with miR-451 but not miR-181a.

FIG. 7A presents the transfection efficiency of erythrocyte cells assessed by the fluorescently labeled oligonucleotides detected by FACS. FIGS. 7B and 7C show the level of parasitemia in the erythrocytes transfected with indicated miRNAs before (left) and after (right) six rounds of propagation. Both miR-223 and miR-451 led to a significant drop in parasitemia.

FIG. 8 provides a PAM analysis of miRNAs separating into two subtypes of SCD. FIG. 8A shows the error plot of PAM analysis with the predictive accuracy at 100% with 6 miRNAs. FIG. 8B presents the expression pattern of the top 13 selected miRNAs separating SCD subtypes. The expression pattern of miR-144, 142_{—}5p in the SCD erythrocyte in microarray (FIG. 8C) as well as further confirmed with real-time RT-PCR (yellow bar) (FIG. 8D).

FIG. 9A presents a comparison of MCV and RNA concentration over time of mature erythrocytes. FIG. 9B shows MCV increases seen for old RBCs enriched with certain miRNAs. FIG. 9C shows that RBC longevity changes due to overexpression of miR-181a.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

It is to be noted that the term “a” or “an” entity refers to one or more of that entity; for example, “a miRNA” is understood to represent one or more miRNAs. As such, the terms “a” (or “an”), “one or more,” and “at least one” can be used interchangeably herein.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

### I. General Overview

Methods to determine the susceptibility of a subject to erythrocyte diseases, including malaria, anemia, and the like are provided. The methods comprise determining the miRNA compositions of erythrocytes from said subject and relating the composition to susceptibility to disease, severity of disease, and the features and clinical phenotypes of the erythrocyte disorders. Additionally, the miRNA composition can be used to predict the stage of the erythrocyte disease and to monitor disease progression and treatment. The present invention is based on the finding that unique miRNA compositions or populations are present in erythrocytes of a patient and these populations can be used to determine susceptibility to erythrocyte disease, severity of erythrocyte disease, and the features and clinical phenotypes of erythrocyte disorders. Additionally, data provided herein demonstrates that particular miRNAs affect the development and progression of various erythrocyte diseases. Thus, the present invention also provides methods for the treatment of subjects with erythrocyte diseases and for preventing the development of the same by administering particular miRNAs or compounds that inhibit the activity of specific miRNAs to the subjects. Pharmaceutical compositions comprising these miRNAs and compounds are also provided herein as well as kits useful for the detection of the miRNAs disclosed herein.

We have found that although mature erythrocytes lack most large-sized RNA, they possess abundant and diverse microRNAs (miRNAs), a class of small-sized RNAs with important regulatory functions (Bartel, D. P. (2004) Cell 116: 281-297). These miRNAs are likely to play important regulatory roles during erythropoeisis. Greater knowledge of the miRNA composition of erythrocytes is likely to provide a unique window on the developmental history and various adaptations during erythropoeisis. The discovery of these expressed miRNAs allows us to use genomic tools and advanced bioinformatics to elucidate the biological role of miRNAs in the erythrocyte, particularly their contribution to the erythrocytes' phenotypic variations. Erythrocytes are the target cells for the asexual stage of malaria parasites. We have found that certain miRNAs are translocated from erythrocytes into malaria parasites and affect malaria growth in erythrocytes. While not bound by any particular mechanism of action it is believed that a different miRNA composition may contribute to the decreased susceptibility to malaria seen in SCD erythrocytes.

Human erythrocyte diseases such as malaria and anemia affect hundreds of millions of people worldwide and impose huge economic and social burdens on the human race. Studies of erythrocyte diseases have led to important knowledge of human genetics and cell biology. For example, sickle cell disease (SCD) was the first characterized single gene disorder, with the identification of the mutation in sickle hemoglobin (HbS) as an A->G nucleotide substitution in the sixth codon of the β-globin gene that results in the substitution of valine for glutamic acid (Pauling et al. (1949) Science 110: 543-548). This mutation reduces the protein solubility and enhances its tendency for polymerization during deoxygenation, resulting in sickling, poor deformability, and the occlusion of microvascular circulation by these polymer-containing erythrocytes (Embury et al. (1994) Sickle Cell Anemia: Basic Principles and Clinical Practice Lippincott Williams & Wilkins. 901 p). SCD is characterized clinically by hemolytic anemia, episodic painful events, chronic organ deterioration and various acute complications (stroke, acute chest syndrome)—many of these clinical manifestations can be attributed to the HbS polymerization (Hillery et al. (2004) Microcirculation 11: 195-208; Serjeant, G. R. (1997) Lancet 350: 725-730; and Stuart et al. (2004) Lancet 364: 1343-1360). Various treatment strategies of SCD, such as HbF induction, are thought to act through the prevention of HbS polymerization (Pace et al. (2006) Dev. Dyn. 235: 1727-1737; and Swank et al. (1998) Curr. Opin. Genet. Dev. 8: 366-370).

It has become clear that the HbS mutation alone cannot account for all symptoms seen in SCD patients. Despite the constant nature of the mutations in homozygous HbSS, the most severe form of SCD, these patients display remarkably variable clinical courses in terms of incidence of painful events, the severity of anemia, the incidence of acute complications (such as stroke and acute chest syndrome), and the frequency of end-organ damage (e.g. heart disease, leg ulcers, stroke) (Gill et al. (1995) Blood 86: 776-783; Castro et al. (1994) Blood 84: 643-649; and Vichinsky et al. (1997) Blood 89: 1787-1792).

While many studies have tried to correlate the risk of specific end organ damage with clinical and laboratory parameters of disease (such as baseline Hb level) and genetic polymorphisms (Steinberg et al. (2006) Curr. Opin. Hematol. 13: 131-136), why such clinical variability occurs among patients with SCD still remains largely a mystery.

Different HbSS patients can have quite a wide range of steady Hb values. Some patients are only able to maintain Hb values of 5.5-6.5 g/dL, while some others maintain Hb values >9 g/dL (West et al. (1992) J. Clin. Epidemiol. 45: 893-909). Such variability is observed even in the absence of α-thalassemia or persistence of fetal Hb (HbF). Therefore, while erythrocyte production rates may vary, it must be assumed that erythrocytes with similar content of HbS nevertheless may have different rates of hemolysis and, consequently, survival. Such differences may arise from variation in the ability of erythrocytes to maintain normal hydration, to avoid adherent interactions, or to modulate other factors that enhance or decrease their survival. Hemolysis leads not only to shortened RBC survival and an incompletely compensated anemia, but also to NO scavenging, superoxide formation, endothelial dysfunction, vasculopathy, increased chance of certain end-organ damages, (Wemmie et al. (2002) Neuron 34: 463-477) and increased risk of death (Fairbrother et al. (2002) Science 297: 1007-1013). The unique clinical characteristics and pathophysiology of these patients has led to the proposal that they belong to a hemolytic subphenotypes of sickle cell disease (Wemmie et al. (2002) Neuron 34: 463-477; and Holtzclaw et al. (2004) Am. J. Respir. Crit. Care Med. 169: 687-695). It remains unknown what accounts for this variability since all HbSS erythrocytes bear the same mutation. Identifying the SCD patients with particular clinical risks will allow us to institute personalized preventive measures to improve the clinical outcomes.

Many epidemiologic studies suggest that the high frequency of HbS alleles amongst people living in malaria-endemic regions reflects the positive selective pressure imposed by the malaria parasite Plasmodium falciparum (Allison, A. C. (1954) Br. Med. J. 1: 290-294; Livincstone, F. B. (1971)Annu. Rev. Genet. 5: 33-64; and Aidoo et al. (2002) Lancet 359: 1311-1312). When infected with P. falciparum, fewer individuals with sickle cell traits (HbSA) die of the infection as compared to normal individuals (Allison, A. C. (1954) Br. Med. J. 1: 290-294; Livincstone, F. B. (1971) Annu. Rev. Genet. 5: 33-64; and Aidoo et al. (2002) Lancet 359: 1311-1312). The mechanism of this resistance remains the subject of considerable debate, but many believe it occurs at the erythrocyte stage of the infection which is due to either extrinsic factors outside of sickle erythrocytes such as enhanced anti-malaria immunity and clearance (Shear et al. (1993) Blood 81: 222-226; Marsh et al. (1989) Trans. R. Soc. Trop. Med. Hyg. 83: 293-303; Abu-Zeid et al. (1992) Trans. R. Soc. Trop. Med. Hyg. 86: 20-22; Bayoumi et al. (1990) Immunol. Lett. 25: 243-249; and Williams et al (2005) PLoS Med. 2: e128) or intrinsic properties of sickle erythrocytes, as manifested by reduced parasite growth or increased erythrocyte sickling (Friedman, M. J. (1978) Proc. Natl. Acad. Sci. USA 75: 1994-1997; Pasvol et al. (1978) Nature 274: 701-703; Luzzatto et al. (1970) Lancet 1: 319-321; and Roth et al. (1978) Science 202: 650-652). It is important to note, however, that malaria resistance is also observed in several other genetic erythrocyte diseases that lack the hallmark erythrocyte sickling (Nagel et al. (1989) Blood 74: 1213-1221).

The establishment of an in vitro culture system of P. falciparum infection in erythrocytes allowed the investigation of the mechanism by which the intrinsic erythrocyte defects enhanced host resistance to malaria (Jensen et al. (1977) J. Parasitol. 63: 883-886). Even though there are some concerns about the possibility of establishing culture conditions faithfully mimicking in vivo biology (Nagel et al. (1989) Blood 74: 1213-1221; and Roth et al. (1989) Science 246: 1051), many studies have clearly shown significant reduction in the growth and replication of P. falciparum in HbSA (from sickle trait individuals) and HbSS erythrocytes compared with normal cells (Friedman, M. J. (1978) Proc. Natl. Acad. Sci. USA 75: 1994-1997; and Pasvol et al. (1978) Nature 274: 701-703). These results suggest the cellular environments themselves of HbSA and HbSS erythrocytes are hostile to the P. falciparum. Importantly, the recapitulation of malaria resistance in vitro allows us to determine how various experimental manipulations will affect the parasite growth to test our hypotheses. Several hypotheses have been proposed to explain the unfavorable cellular environment, including low K+ content (Friedman et al. (1979) Exp. Parasitol. 47: 73-80), heme toxicity (Orjih et al. (1985) Am. J. Trop. Med. Hyg. 34: 223-227; and Orjih et al. (1981) Science 214: 667-669), oxidant damage (Friedman, M. J. (1979) Nature 280: 245-247) and polymerized hemoglobin (Eaton et al. (1987) Blood 70: 1245-1266). But, it is still unknown what cellular characteristics in the sickle erythrocytes are responsible for their relative resistance to Plasmodium infection observed in vitro. Understanding the molecular basis of this resistance will not only lead to better explanation of this powerful selection force of malaria, but also may potentially lead to novel approaches to prevention and treatment of malaria.

Similar to SCD, human cancers also exhibit exceedingly complex phenotypes. The characteristics of an individual tumor have an enormous phenotypic complexity—a result of multiple mutations in oncogenes and tumor suppressors, varying environmental conditions, and a huge range of inherited germline variations. While the effect of any one of these genetic alterations and environmental factors may be quite subtle, their combined effects can make an important contribution to the tumor phenotype, leading to immense natural heterogeneity in tumor phenotypes, disease outcomes, and response to therapies. Traditional analysis cannot capture the complexity of the actual disease process and results in broad categorizations that are often imprecise for the individual tumor or patient. Advances in genomic technologies, such as microarray, over the past several years have provided an opportunity for more precise characterizations of the tumor and the patient (Alizadeh et al. (2000) Nature 403: 503-511; Huang et al. (2003) Lancet 361: 1590-1596; Perou et al. (2000) Nature 406: 747-752; Golub, T. R. (2001) N. Engl. J. Med. 344: 601-602; Golub, T. R. (2004) Cancer Cell 6: 107-108; Golub, T. R. (1999) Science 286: 531-537; van de Vijver et al. (2002) N. Engl. J. Med. 347: 1999-2009; and van't Veer et al. (2002) Nature 415: 530-536). Not only do these measures allow for an assay of the activity of essentially all genes within the genome, the much more powerful aspect is the ability to use the information to identify patterns, or profiles, of gene activity that characterize a given phenotype. Several relevant important themes coming from the genomic analysis have become instructive: 1) the global gene expression can be used for “class discovery” to identify previously unknown subtypes, such as was done in large-cell lymphoma (Alizadeh et al. (2000) Nature 403: 503-511), breast (Perou et al. (2000) Nature 406: 747-752), and lung cancers (Garber et al. (2001) Proc. Natl. Acad. Sci. USA 98: 13784-13789); 2) it is possible to select a small subset of genes from a whole array to distinguish tumor subtypes with significant differences in clinical phenotypes for personalized treatment plans, such as breast (van de Vijver et al. (2002) N. Engl. J. Med. 347: 1999-2009; and van't Veer et al. (2002) Nature 415: 530-536), and lung cancers (Potti et al. (2006) N. Engl. J. Med. 355: 570-580; and Chen et al. (2007) N. Engl. J. Med. 356: 11-20); 3) the genes identified using microarrays can be translated into clinically-useable tests based on real-time PCR (Lossos et al. (2004) N. Engl. J. Med. 350: 1828-1837) or other assays; 4) the analysis of global miRNA expression pattern has led to the identification of important clinical subtypes and molecular features of human cancers (Lu et al. (2005) Nature 435: 834-838; Calin et al. (2005) N. Engl. J. Med. 353: 1793-1801; and Voorhoeve et al. (2006) Cell 124: 1169-1181).

Although this analytic framework for complex tumor phenotypes has made significant impact on many other human diseases, its application to red blood cell disorders has been limited. The transcriptome analysis of erythroid biology has focused on the change of global gene expression in cultured cells undergoing differentiation (Keller et al. (2006) Physiol. Genomics 28: 114-128; Addya et al. (2004) Physiol. Genomics 19: 117-130; and Heo et al. (2005) Mol. Cells. 20: 57-68) or exposed to hydroxyurea (Wang et al. (2002) Br. J. Haematol. 119: 1098-1105) and the gene expression pattern of white blood cells in various erythrocyte diseases (Holtzclaw et al. (2004) Am. J. Respir. Crit. Care. Med. 169: 687-695; Nagel et al. (1989) Blood 74: 1213-1221; Jison et al. (2004) Blood 104: 270-280; Rybicki et al. (2003) Blood Cells Mol. Dis. 31: 370-380; Goerttler et al. (2005) Br. J. Haematol. 129: 138-150; Ebert et al. (2005) Blood 105: 4620-4626; Qian et al. (2005) Oncol. Rep. 14: 1189-1197; Pellagatti et al. (2004) Br. J. Haematol. 125: 576-583; and Ueda et al. (2003) Br. J. Haematol. 123: 288-296). Since it is commonly believed that reticulocytes are the last stage of erythroid development which still retains mRNA expression, several studies have focused on the global expression pattern of reticulocytes (Goh et al. (2004) Nucleic Acids Res. 32: D572-574; Miller, J. L. (2004) Blood Cells Mol. Dis. 32: 341-343; Lee et al. (2001) Blood 98: 1914-1921; and Gubin et al. (1999) Genomics 59: 168-177). One important gap in these studies is the lack of attention to the mature erythrocyte, the main cell type affected by these red blood cell diseases. Given the long-held view that mature erythrocytes do not contain nucleic acids, the transcriptome analysis of mature erythrocytes has been lacking. Several studies have attempted to use proteomic and metabolomic techniques to characterize the whole composition of protein in mature erythrocytes (Pasini et al. (2006) Blood 108: 791-801; Brand et al. (2004) Nat. Struct. Mol. Biol. 11: 73-80; Thadikkaran et al. (2005) Proteomics 5: 3019-3034; and Jamshidi et al. (2006) Blood Cells Mol. Dis. 36: 239-247) or anemia disorders (Orrú et al. (2007) Mol. Cell. Proteomics 6: 382-393; and Kakhniashvili et al. (2005) Exp. Biol. Med. (Maywood) 230: 787-792). These approaches hold great long term potential, but their technical development and analytic methods still lag behind those of DNA microarrays.

Human mature erythrocytes are terminally differentiated cells that have lost nuclei and all cellular organelles. These cells make up more than 90% of the cell population in the peripheral blood and are end-products of a highly regulated differentiation process that involves the gradual loss of cellular organelles, a decline in nucleic acid content, and a step-wise acquisition of erythrocyte characteristics (Hoffman et al. (2004) Hematology: Basic Principles And Practice: Churchill Livingstone). One striking feature of erythroid differentiation is that the nuclei are extruded from cells as they differentiate into reticulocytes, the immediate precursor of mature erythrocytes. Cytoplasmic RNA and translation activities are still detectable in reticulocytes, but fall below the detection limit as reticulocytes terminally differentiate to become mature erythrocytes (Goh et al. (2004) Nucleic Acids Res. 32: D572-574). The prevailing view that mature erythrocytes lack most RNAs primarily comes from their inability to stain with RNA-binding dyes (such as thiazole orange or methylene blue), which is the basis of the clinical utility of these dyes to distinguish reticulocytes from mature erythrocytes (Lee, L. G. (1986) Cytometry 7: 508-517). Given the potential limitation and biases of these approaches to detect RNA molecules, certain RNA species may not be identified and characterized. In the present invention, we have found that human mature erythrocytes, although largely lacking in ribosomal and large-sized RNA, contain diverse and abundant miRNAs not recognized before. Having discovered the presence of these miRNA in mature erythrocytes, one of skill in the art can apply DNA microarray technology and other molecular techniques to capture and extract the biological information to understand erythrocyte phenotypes during physiological and pathological adaptations. Thus, miRNA compositions that indicate the susceptibility or presence of a disease state, particularly sickle cell disease, anemia disorders, malaria, and other red blood cell (erythrocyte) diseases are encompassed.

The Regulatory Roles of miRNAs—in General and in Erythroid Biology

mRNAs are non-coding RNA of 19-25 nt in size which mediate post-transcriptional regulation of their target mRNA through the formation of non-canonical base-pairing with the 3′UTR. MiRNAs have been shown to modulate the post-transcriptional control of their target mRNA, thereby regulating a wide variety of biological processes (e.g. differentiation, apoptosis, oncogenic transformation) (Bartel D P (2004) Cell 116: 281-297). The global miRNA expression pattern correlates with important biological and clinical phenotypes of human cancers in many studies (Lu et al. (2005) Nature 435: 834-838; Calin et al. (2005) N. Engl. J. Med. 353: 1793-1801; Voorhoeve et al. (2006) Cell 124: 1169-1181; Esquela-Kerscher et al. (2006) Nat. Rev. Cancer 6: 259-269; and Yanaihara et al. (2006) Cancer Cell 9: 189-198). Several miRNAs have been also implicated in the process of erythropoeisis—miR-221/mir-222 are involved in downregulating kit receptor, an important step essential for successful erythrocyte differentiation (Felli et al. (2005) Proc. Natl. Acad. Sci. USA 102: 18081-18086). Current predictive algorithms (Lewis et al. (2005) Cell 120: 15-20; and Krek et al. (2005) Nat. Genet. 37: 495-500) identify the 3′UTR of many important regulatory genes (e.g. GATA-1, EKLF) in erythropoeisis as containing many potential targeted sites for miRNAs.

Since erythroid cells lose their nuclei and active transcription during the reticulocyte stages of their development, it is believed that post-transcriptional regulation of remaining mRNA plays a very important role, such as the regulation of iron regulatory proteins (Rouault, T. A. (2006) Nat. Chem. Biol. 2: 406-414) and 15-lipoxygenase (Ostareck et al. (2001) Cell 104: 281-290; Ostareck et al. (1997) Cell 89: 597-606; and Gebauer et al. (2001) Cold Spring Harb. Symp. Quant. Biol. 66: 329-336). The constant spring thalassemia (α^{cs}) disorder is caused by a point mutation in the globin 3′UTR that affects the stability of its mRNA (Waggoner et al. (2003) Exp. Biol. Med. (Maywood) 228: 387-395). The induction of HbF expression by sodium butyrate has been shown to occur at the translation level (Weinberg et al. (2005) Blood 105: 1807-1809). Given our current understanding of miRNAs, it is believed that they play a regulatory role in post-transcriptional regulation in the erythroid cells. Given the easy access to a large number of cells, studying miRNAs in the mature erythrocyte may provide a unique window into understanding the regulatory roles of miRNAs during the physiological and pathological manifestation of erythrocyte diseases.

### II. Methods

The present invention is drawn to methods for determining the susceptibility of a subject to an erythrocyte disease, for determining the severity of an erythrocyte disease, or for monitoring an erythrocyte disease based on the composition of miRNA present in the erythrocyte of a subject. Methods for the treatment of a subject with an erythrocyte disease and for the identification of erythrocyte miRNAs associated with particular erythrocyte diseases are also provided herein.

A. Methods for Determining the Susceptibility of a Subject to an Erythrocyte Disease

Methods for determining the susceptibility to an erythrocyte disease in a subject comprise obtaining a sample of erythrocytes from the subject and determining the composition of miRNA present in the erythrocytes wherein the composition of miRNA is predictive of susceptibility to an erythrocyte disease.

By subject or patient is intended an animal, including a mammal, such as a human, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use. In particular embodiments, the subject is a human.

The term “erythrocyte” refers to a mature red blood cell that is CD71⁻. The term “reticulocyte” refers to an immature red blood cell that is characterized by the cellular surface expression of CD71 (CD71⁺). Thus, the term “red blood cell” can refer to either a mature red blood cell (i.e., erythrocyte) or an immature red blood cell (i.e., reticulocyte). Under normal physiological conditions, reticulocytes generally represent a minor fraction of red blood cells throughout the body. Reticulocytes differentiate into mature erythrocytes, which make up the majority of the cells in the blood, typically having a life span of about 120 days.

Erythrocytes can be obtained from a subject using any suitable purification method known in the art to isolate the erythrocytes from whole blood, including but not limited to density gradient purification, FACS, filtration, and antibody depletion. In certain embodiments, the erythrocyte purification scheme described elsewhere herein (see the materials and methods section within the Experimental section) can be used to isolate erythrocytes. In some embodiments, erythrocytes are substantially pure and are substantially free from platelets, leukocytes, or reticulocytes. Thus, in some embodiments, the percentage of reticulocytes found within the purified erythrocyte population comprises less than 10%, less than 5%, less than 4%, less than 3%, less than 2%, less than 1.5%, less than 1%, less than 0.5%, or lower of the total cell population.

Total RNA can be isolated from the purified population of erythrocytes. General methods for RNA extraction are well known in the art and are disclosed in standard textbooks of molecular biology, including Ausubel et al., ed., Current Protocols in Molecular Biology, John Wiley & Sons, New York 1987-1999. In particular, RNA isolation can be performed using the mirVana microRNA isolation kit, which is commercially available from Ambion (Austin, Tex.), according to the manufacturer's instructions. This kit allows the capture of RNAs as small as 10 nucleotides. In some embodiments, the isolated total RNA can be size-fractionated using methods known in the art to enrich the population of RNAs for RNAs of a small size. In certain embodiments, the isolated (and in some embodiments, size-fractionated) RNA population is enriched for RNAs that have a length of less than 400 bp, less than 300 bp, less than 200 bp, less than 100 bp, less than 50 bp, less than 40 bp, less than 30 bp, less than 25 bp, or less than 20 bp.

Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the miRNA being detected. The nucleic acid probe can be of sufficient length and specificity to specifically hybridize under stringent conditions to a miRNA of the present invention, or any derivative DNA or RNA. Hybridization of a miRNA with the probe indicates that the miRNA in question is present.

In one embodiment, the miRNA is immobilized on a solid surface and contacted with a probe, for example by running the isolated RNA on an agarose gel and transferring the RNA from the gel to a membrane, such as nitrocellulose. In an alternative embodiment, the probes are immobilized on a solid surface and the RNA is contacted with the probes, for example, in an array. A skilled artisan can readily adapt known miRNA detection methods for use in detecting the level of miRNAs useful for the present invention.

An alternative method for determining the level of a miRNA in a sample involves the process of nucleic acid amplification, for example, by RT-PCR (U.S. Pat. No. 4,683,202), ligase chain reaction (Barany (1991) Proc. Natl. Acad. Sci. USA 88:189-93), self sustained sequence replication (Guatelli et al. (1990) Proc. Natl. Acad. Sci. USA 87:1874-78), transcriptional amplification system (Kwoh et al. (1989) Proc. Natl. Acad. Sci. USA 86:1173-77), Q-Beta Replicase (Lizardi et al. (1988) Bio/Technology 6:1197), rolling circle replication (U.S. Pat. No. 5,854,033), or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, miRNA levels are assessed by quantitative RT-PCR. In some embodiments, a multiplexing quantitative PCR assay, such as a stem-loop RT-PCR assay, such as that described previously (Lao et al. (2006) Biochem. Biophys. Res. Commun. 343:85-89) and elsewhere herein (see Experimental Example 1), is used to assess the levels of miRNAs. For PCR analysis, well known methods are available in the art for the determination of primer sequences for use in the analysis.

MicroRNA microarrays provide one method for the simultaneous measurement of the expression levels of multiple miRNAs. Each array consists of a reproducible pattern of capture probes attached to a solid support. Labeled RNA is hybridized to complementary probes on the array and then detected by laser scanning. Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the miRNA expression profile for a large number of miRNAs in a sample.

Techniques for the synthesis of these arrays using mechanical synthesis methods are described in, for example, U.S. Pat. No. 5,384,261. Although a planar array surface is generally used, the array can be fabricated on a surface of virtually any shape or even a multiplicity of surfaces. Arrays can be nucleic acids on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992. Arrays can be packaged in such a manner as to allow for diagnostics or other manipulation of an all-inclusive device. See, for example, U.S. Pat. Nos. 5,856,174 and 5,922,591.

In a specific embodiment of the microarray technique, oligonucleoties with sequences that are complementary to miRNAs are applied to a substrate in a dense array. The microarrayed oligonucleotides, immobilized on the microchip, are suitable for hybridization under stringent conditions. Fluorescently labeled miRNAs can be generated through incorporation of fluorescent nucleotides using any method known to one of skill in the art. In particular embodiments, the miRNAs are labeled using the mirVana miRNA labeling kit that is commercially available from Ambion and amine-reactive dyes according to the manufacturer's instructions. Alternatively, in other embodiments, the miRNAs are labeled with the mercury LNA Array Labeling Kit from Exiqon (Vedbaek, Denmark) according to the manufacturer's instructions. Labeled miRNAs applied to the chip hybridize with specificity to each spot comprising a complementary oligonucleotide on the array. After stringent washing to remove non-specifically bound RNA, the chip is scanned by confocal laser microscopy or by another detection method, such as a CCD camera. Quantitation of hybridization of each arrayed element allows for assessment of corresponding mRNA abundance.

With dual color fluorescence, separately labeled miRNAs generated from two sources of RNA are hybridized pairwise to the array. The relative abundance of the miRNAs from the two sources corresponding to each specified miRNA is thus determined simultaneously. The miniaturized scale of the hybridization affords a convenient and rapid evaluation of the expression pattern for large numbers of miRNAs. Such methods have been shown to have the sensitivity required to detect low levels of miRNA, and to reproducibly detect at least approximately two-fold differences in the expression levels (Schena et al. (1996) Proc. Natl. Acad. Sci. USA 93: 106-49). Microarray analysis can be performed by commercially available equipment, following manufacturer's protocols, such as by using the Affymetrix GenChip technology, or Agilent ink-jet microarray technology. The development of microarray methods for large-scale analysis of miRNA levels makes it possible to identify the miRNA composition of erythrocyte samples from various subjects.

To aid in the interpretation of the results from a miRNA microarray, the data can be normalized. “Normalization” may be used to remove sample-to-sample variation. For microarray data, the process of normalization aims to remove systematic errors by balancing the fluorescence intensities of the two labeling dyes. The dye bias can come from various sources including differences in dye labeling efficiencies, heat and light sensitivities, as well as scanner settings for scanning two channels. Some commonly used methods for calculating normalization factor include: (i) global normalization that uses all miRNAs on the array; (ii) normalization RNA normalization, wherein the normalization RNA is constantly expressed; and (iii) internal controls normalization that uses known amount of miRNAs added during hybridization (Quackenbush (2002) Nat. Genet. 32 (Suppl.), 496-501). In one embodiment, the miRNAs disclosed herein can be normalized to at least one normalization RNA, which is an RNA whose level is constant and abundantly expressed across multiple tissues, such as U6 snoRNA. In certain embodiments, the normalization RNA is a miRNA whose level does not change throughout erythroid differentiation, including but not limited to hsa-miR-152. It will be understood by one of skill in the art that the methods disclosed herein are not bound by normalization to any particular normalization RNA, and that any suitable normalization RNA known in the art can be used. In some embodiments, the data is normalized to the geometric mean of a set of multiple normalization RNAs.

Similarly, the level of a particular miRNA can be measured using a real-time RT-PCR assay, such as the stem-loop RT-PCR assay. The real-time PCR data can also be normalized to at least one normalization gene.

By “miRNA composition” or “composition of miRNA” is intended a single miRNA (as defined elsewhere herein) or a population of miRNAs present within a single cell or a population of cells (of the same or different type, e.g., different differentiation state, different disease states, from different tissues, different cell lines). The population of cells from which the miRNA composition is determined can be from the same or different subjects. The composition of miRNA from a cell or population of cells can be determined using any of the techniques referred to immediately above. In some embodiments, the miRNA composition is determined using a microRNA microarray or a multiplexing RT-PCR assay.

The subject can be afflicted with an erythrocyte disease, can be suspected of having an erythrocyte disease, or may be a healthy individual wanting to assess their susceptibility to an erythrocyte disease. As used herein, an “erythrocyte disease” refers to any type of pathological condition that affects red blood cells, including but not limited to, anemia, a sickle cell disease, and malaria.

The presently disclosed methods can be used to assess a subject's susceptibility to an erythrocyte disease. As used herein, the term “susceptibility” refers to the likelihood that the subject has the disease in question (clinical or subclinical), will contract or develop the disease at any point during the subject's lifetime. In some cases wherein a subject's susceptibility to disease refers to the likelihood that the subject has the disease in question, the patient may not be presenting with clinical symptoms typically associated with the disease at the time the subject's susceptibility to the disease is being assessed and thus has a subclinical form of the disease.

In some embodiments, the erythrocyte disease is anemia. Anemia is characterized by abnormally low levels of healthy red blood cells or red blood cells with low levels of hemoglobin or with mutated hemoglobin. Anemias can include, for example, drug-(chemotherapy-) induced anemias, hemolytic anemias due to hereditary cell membrane abnormalities, such as hereditary spherocytosis, hereditary elliptocytosis, and hereditary pyropoikilocytosis; hemolytic anemias due to acquired cell membrane defects, such as paroxysmal nocturnal hemoglobinuria and spur cell anemia; hemolytic anemias caused by antibody reactions, for example to the RBC antigens, or antigens of the ABO system, Lewis system, Ii system, Rh system, Kidd system, Duffy system, and Kell system; methemoglobinemia; a failure of erythropoiesis, for example, as a result of aplastic anemia, pure red cell aplasia, myelodysplastic syndromes, sideroblastic anemias, and congenital dyserythropoietic anemia; secondary anemia in non-hematolic disorders, for example, as a result of chemotherapy, alcoholism, or liver disease; anemia of chronic disease, such as chronic renal failure; and endocrine deficiency diseases. The anemia can be associated with sickle cell disease (SCD). In those embodiments wherein the erythrocyte disease comprises anemia, the composition of miRNAs within the erythrocytes can comprise at least one of miR-144 and miR-142-5p, wherein the level of at least one of miR-144 and miR-142-5p is positively correlated with the susceptibility to anemia (e.g., hemolytic anemia).

A positive correlation defines a relationship between two variables, wherein a change in one variable in one direction (e.g., increase or decrease) results in a change in the second variable in the same direction. For example, the level of a particular miRNA is positively correlated with the severity of a certain erythrocyte disease if an increase in the level of the miRNA is associated with an increase in the severity of the disease and conversely, a decrease in the level of the miRNA is associated with a decrease in the severity of the disease.

On the other hand, a negative correlation defines a relationship between two variables, wherein a change in one variable in one direction results in a change in the second variable in the opposite direction. Thus, the level of a particular miRNA is negatively correlated with the severity of a certain erythrocyte disease if an increase in the level of the miRNA is associated with a decrease in the severity of the disease and conversely, a decrease in the level of the miRNA is associated with an increase in the severity of the disease.

A positive or negative correlation between a particular miRNA or set of miRNAs and the severity or susceptibility of an erythrocyte disease can be determined by analyzing the levels of the miRNA or set of miRNAs within a population of subjects with and without the disease and with various gradations of severity of the disease using, for example, an analysis of microRNA microarray data such as those described elsewhere herein (see Experimental section).

Thus, in some embodiments, increased levels of miR-144 and miR-142-5p can indicate an enhanced susceptibility to anemia when compared to a control. Thus, in those embodiments wherein the erythrocytes of the subject have an increased level of at least one of miR-144 and miR-142-5p over that of a control, the subject has an increased chance of developing anemia or of exhibiting a more severe form of anemia relative to the control. Conversely, a reduction in the level of at least one of miR-144 and miR-142-5p compared to a control can indicate a reduced susceptibility to anemia relative to the control. Thus, the levels of miR-144 and miR-142-5p are positively correlated with susceptibility to anemia.

To determine a subject's susceptibility to an erythrocyte disease, the level of the subject's erythrocyte miRNA can be compared to a control. In some instances, the control can be one or more subjects not having or not suspected of having the erythrocyte disease or the control can be a previously assayed value for the same subject. In other instances, for example, wherein one is classifying a subtype of a particular erythrocyte disease, wherein a subtype is associated with a more severe erythrocyte disease than other subtypes and the subtypes can be classified based on the level of a particular miRNA in these patients, the control would be the average value of the level of the miRNA across a population of patients with the erythrocyte disease that one is attempting to classify. In those embodiments wherein one is determining the severity of a disease relative to a control, a control may comprise one or more patients with the erythrocyte disease that exhibit more or less severe symptoms that would indicate a more or less severe disease.

In some embodiments, the increase in the level of at least one of miR-144 and miR-142-5p over that of a control that indicates an enhanced susceptibility to anemia, or the reduction in the level of at least one of miR-144 and miR-142-5p over that of a control that indicates a reduced susceptibility to anemia can be a fold change greater than 1, including but not limited to, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 10-fold, at least 50-fold, at least 100-fold, at least 1000-fold, or greater when compared to a control.

Also provided herein are methods for determining the susceptibility to malaria in a subject comprising obtaining a sample of erythrocytes from the subject and determining the composition of miRNA present in the erythrocytes wherein the composition of miRNA is predictive of susceptibility to malaria.

In some of these embodiments, the composition of miRNA comprises a miRNA capable of translocating into a malaria parasite. By translocate when referring to a pathogen is intended the transfer of a host material (e.g., miRNA) from the host into a pathogen infecting the host. The miRNA can translocate into the parasite during any stage of infection, particularly later stages of infection. The ability of a miRNA to translocate into the parasite can be determined using assays presented elsewhere herein (see Experimental Example 2). The translocated miRNA can inhibit the growth or survival of the parasite, in which case, the levels of these miRNAs in the erythrocyte are then positively correlated with the susceptibility to malaria. Malaria parasites include the protozoan parasites belonging to the Plasmodium genus, including but not limited to Plasmodium falciparum, Plasmodium vivax, Plasmodium ovale, Plasmodium malariae, Plasmodium reichenowi, that are capable of invading a subject's red blood cells, leading to the clinical, molecular, and cellular hallmarks of malaria. In some embodiments, the malaria parasite comprises Plasmodium falciparum.

In certain embodiments, the miRNA that is capable of translocating into the parasite comprises those miRNAs described elsewhere herein, including miR-451 and miR-223 (see Experimental Example 2). Erythrocytes overexpressing miR-451 or miR-223 exhibit a reduced parasitemia when infected with P. falciparum (see Experimental Example 2). Thus, a subject that comprises an increased level of at least one of miR-451 and miR-223 in the erythrocytes compared to a control has a decreased susceptibility to malaria relative to the control. Conversely, a subject that has a reduced level of at least one of miR-451 and miR-223 in the erythrocytes compared to a control has an enhanced susceptibility to malaria relative to the control. Thus, the levels of miR-451 and miR-223 are negatively correlated with susceptibility to malaria. In particular embodiments, the subject has sickle-cell disease (e.g., HbSS) or sickle-cell trait (HbSA).

In some embodiments, the increase in the level of at least one of miR-451 and miR-223 over that of a control that indicates a reduced susceptibility to malaria, or the reduction in the level of at least one of miR-451 and miR-223 over that of a control that indicates an enhanced susceptibility to malaria relative to the control can be a fold change greater than one, including but not limited to at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 10-fold, at least 50-fold, at least 100-fold, at least 1000-fold, or greater.

B. Methods for Determining the Severity of an Erythrocyte Disease and for Monitoring an Erythrocyte Disease

Methods for determining the severity of an erythrocyte disease or monitoring an erythrocyte disease comprise obtaining a sample of erythrocytes from a subject and determining the composition of miRNA present in the erythrocytes, wherein the composition of miRNA is predictive of the severity or of the disease state of the erythrocyte disease. In some of these embodiments, the erythrocyte disease is sickle-cell disease (SCD). Patients with sickle-cell disease have at least one copy of the sickle hemoglobin (HbS) allele, which is an A->G nucleotide substitution in the sixth codon in the β-globin gene that results in the substitution of valine for glutamic acid (Pauling et al. (1949) Science 110: 543-548) and one other defective mutation, either in a globin gene, such as in individuals who are homozygous for the sickle hemoglobin allele (HbSS) or in another gene that affects the red blood cell, leading to the characteristic sickling of the red blood cells. As used herein, a “sickle cell” includes a cell which is an abnormal, crescent-shaped erythrocyte that contains sickle cell hemoglobin. “Sickling” includes the process whereby a normal-shaped cell becomes crescent-shaped.

Thus, a subject having sickle cell disease includes those subjects that are homozygous for the sickle hemoglobin allele (i.e., have an HbSS genotype, which is the most severe sickle cell disease), an HbSC genotype, resulting in sickle-hemoglobin C disease, or those that have sickle-hemoglobin E disease or hemoglobin S-beta thalassemia. SCD is characterized clinically by hemolytic anemia, episodic painful events, chronic organ deterioration and various acute complications (stroke, acute chest syndrome) (Hillery et al. (2004) Microcirculation 11: 195-208; Serjeant, G. R. (1997) Lancet 350: 725-730; and Stuart et al. (2004) Lancet 364: 1343-1360).

Subjects with one HbS allele and one normal allele (HbSA) have sickle cell trait, which is not regarded as a disease state because the complications are either uncommon or mild. Nevertheless, patients with HbSA still have the problem of polymerization of deoxy-Hb S since serious morbidity or mortality can result from complications with processes that cause hypoxia, acidosis, dehydration, hyperosmolality, hypothermia, or elevated erythrocyte 2,3-DPG. These conditions can transform a silent sickle cell trait into a syndrome resembling sickle cell disease with vaso-occlusion due to rigid erythrocytes (Embury et al. (1994) Sickle Cell Anemia: Basic Principles and Clinical Practice: Lippincott Williams & Wilkins. 901 p).

In some methods for determining the severity of an erythrocyte disease or for monitoring an erythrocyte disease wherein the erythrocyte disease is a sickle cell disease, the composition of miRNA present in the erythrocytes comprises a reticulocyte-specific miRNA, wherein the level of the reticulocyte-specific miRNA is positively correlated with the severity or progression of the sickle cell disease. Reticulocyte-specific miRNAs, as presented elsewhere herein (see Experimental Example 1), are those miRNAs that are present at higher levels in reticulocytes than in erythrocytes. Thus, the levels of a reticulocyte-specific miRNA in a reticulocyte as compared with an erythrocyte can have a fold change of greater than 1, including but not limited to, at least 1.1-fold higher, at least 1.2-fold higher, at least 1.3-fold higher, at least 1.4-fold higher, at least 1.5-fold higher, at least 1.6-fold higher, at least 1.7-fold higher, at least 1.8-fold higher, at least 1.9-fold higher, at least 2-fold higher, at least 2.5-fold higher, at least 3-fold higher, at least 3.5-fold higher, at least 4-fold higher, at least 4.5-fold higher, at least 5-fold higher, at least 10-fold higher, at least 50-fold higher, at least 100-fold higher, at least 1000-fold higher, or greater than an erythrocyte. A representative list of reticulocyte-specific miRNAs is provided in Table 1. In some embodiments, the subject exhibits an increase in the level of a single reticulocyte-specific miRNA and in other embodiments, the subject exhibits an increase in a population of different reticulocyte-specific miRNAs, including but not limited to those provided in Table 1. As noted above, the levels of the miRNAs can be normalized to a normalization RNA.

The severity of a subject's disease generally refers to the level and frequency of disease-associated symptoms and the progression of the disease overall. The severity of a particular subject's disease can be assessed using any type of diagnostic and prognostic procedures known in the art. Thus, a more severe or more progressive sickle-cell disease is one wherein the disease state is more advanced and the patient exhibits more or more frequent clinical manifestations of the disease or a more severe phenotype (e.g., characterized by higher levels of hemolysis).

In some embodiments, methods for determining the severity of a sickle cell disease in a subject with sickle cell disease comprise determining the composition of miRNA present in erythrocytes from the subject, wherein the miRNA composition comprises at least one of miR-144 and miR-142-5p, and wherein the level of at least one of miR-144 and miR-142-5p is positively correlated with the severity or progression of the sickle cell disease. Thus, an increase in the levels of at least one of miR-144 and miR-142-5p compared to a control can indicate a more severe or more progressive sickle-cell disease relative to the control, whereas a decrease in the levels of at least one of miR-144 and miR-142-5p compared to a control can indicate a less severe or less progressive sickle-cell disease relative to the control.

Levels of miR-144 and miR-142-5p can be used to classify subjects with various subtypes of erythrocyte diseases. For example, levels of miR-144 and/or miR-142-5p can classify subjects with SCD (e.g., HbSS) or sickle cell trait (HbSA) into subtypes based on their susceptibility to or severity of anemia (e.g., hemolytic anemia), wherein a relatively high level of at least one of miR-144 and miR-142-5p correlates with a more severe disease.

In other embodiments, methods for determining the severity of a sickle-cell disease or monitoring the progression of the disease in a subject include determining the composition of miRNA present in erythrocytes from the subject, wherein the composition comprises at least one of miR-320, miR-23b, and miR-221, and wherein the level of at least one of miR-320, miR-23b, and miR-221 is negatively correlated with the severity or progression of the sickle-cell disease, such that an increase in the level of at least one of miR-320, miR-23b, and miR-221 compared with a control indicates a less severe or less progressive sickle-cell disease relative to the control. Conversely, a decrease in the level of at least one of miR-320, miR-23b, and miR-221 compared with a control indicates a more severe or more progressive sickle-cell disease relative to the control. As described elsewhere herein (see Experimental Example 1), miR-320 targets the reticulocyte marker CD71 and while not bound by any particular theory or mechanism, a decrease in the level of miR-320 indicates a dysregulation in terminal differentiation of red blood cells and a younger age of circulating erythrocytes. In addition, the inhibition of miR-320 leads to a reduction in cell survival and hemolysis.

Also noted elsewhere herein (see Experimental Example 1), miR-23b and miR-221 positively correlate with HbF expression, which can reduce the severity of the sickle-cell phenotype. Thus, decreased levels of miR-23b and miR-221 are associated with a more severe or more progressive disease relative to a control.

The decrease in the level of at least one of miR-320, miR-23b, and miR-221 that indicates a more severe or more progressive disease or the increase in the level of at least one of miR-320, miR-23b, and miR-221 that indicates a less severe or more progressive disease can be a fold change greater than 1, including but not limited to at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 10-fold, at least 50-fold, at least 100-fold, at least 1000-fold, or greater when compared with a control.

C. Methods for Increasing the Life-Span of Erythrocytes

In other embodiments of the invention, the miRNAs or a miRNA composition can be used to increase the life-span of an erythrocyte. In particular, introducing a miRNA into an erythrocyte leads to an increase in survival. In some embodiments, the erythrocyte is an old erythrocyte. By “old” erythrocyte is intended an erythrocyte near the end of the 120 day life-span, generally at least 100 days, at least 105 days, at least 110 days, at least 115 days. An increase in survival means that the erythrocyte is able to survive beyond the 120 day life-span to at least 130 days, at least 140 days, at least 150 days, at least 160 days, at least 170 days, at least 180 days or longer. A mixture of miRNAs or a single population of miRNAs can be introduced into the erythrocyte. In particular, the miRNA that is introduced into an erythrocyte comprises at least one of miR-181a, Let-7a-1, and miR-17. In certain embodiments, the miRNA comprises miR-181. The sequence of miR-181a, Let-7a-1, and miR-17 are set forth in SEQ ID NOs: 7, 8, and 9, respectively. Thus, in some embodiments, methods for increasing the life-span of an erythrocyte comprise introducing into the erythrocyte a polynucleotide comprising or encoding a miRNA or a precursor thereof, wherein said miRNA has a nucleotide sequence that has at least 80%, at least 85%, at least 90%, at least 95%, at least 96%, at least 97%, at least 98%, at least 99%, or 100% sequence identity to SEQ ID NO: 7. In other embodiments, a polynucleotide is introduced into the erythrocyte, wherein the polynucleotide comprises or encodes a miRNA or a precursor thereof, wherein said miRNA has a nucleotide sequence that has at least 80%, at least 85%, at least 90%, at least 95%, at least 96%, at least 97%, at least 98%, at least 99%, or 100% sequence identity to SEQ ID NO: 8. In further embodiments, a polynucleotide is introduced into the erythrocyte, wherein the polynucleotide comprises or encodes a miRNA or a precursor thereof, wherein the miRNA has a nucleotide sequence that has at least 80%, at least 85%, at least 90%, at least 95%, at least 96%, at least 97%, at least 98%, at least 99%, or 100% sequence identity to SEQ ID NO: 9. In some embodiments, the miRNA or precursor thereof comprises the first 8 nucleotides of the sequence set forth in SEQ ID NO:7, 8, or 9.

The term “polynucleotide” is intended to encompass a singular nucleic acid, as well as plural nucleic acids, and refers to a nucleic acid molecule or construct, e.g., messenger RNA (mRNA), plasmid DNA (pDNA), or short interfering RNA (siRNA). A polynucleotide can be single-stranded or double-stranded, linear or circular. A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. The term “polynucleotide” can refer to an isolated nucleic acid or polynucleotide, wherein by “isolated” nucleic acid or polynucleotide is intended a nucleic acid molecule, DNA or RNA, that has been removed from its native environment. Examples of an isolated polynucleotide include recombinant polynucleotides maintained in heterologous host cells or purified (partially or substantially) polynucleotides in solution. Isolated polynucleotides or nucleic acids according to the present invention further include such molecules produced synthetically. Isolated polynucleotides also can include isolated expression vectors, expression constructs, or populations thereof. “Polynucleotide” also can refer to amplified products of itself, as in a polymerase chain reaction. The “polynucleotide” can contain modified nucleic acids, such as phosphorothioate, phosphate, ring atom modified derivatives, and the like. The “polynucleotide” can be a naturally occurring polynucleotide (i.e., one existing in nature without human intervention), or a recombinant polynucleotide (i.e., one existing only with human intervention). While the terms “polynucleotide” and “oligonucleotide” both refer to a polymer of nucleotides, as used herein, an oligonucleotide is typically less than 100 nucleotides in length.

The methods presented herein require introducing a polynucleotide into an erythrocyte. “Introducing” is intended to mean presenting to a cell the polynucleotide in such a manner that the sequence gains access to the interior of the cell. Such introduction can be carried out by any method known in the art, including but not limited to transfection, electroporation, microinjection, infection with a viral or bacteriophage vector containing the polynucleotide sequences, cell fusion, chromosome-mediated gene transfer, microcell-mediated gene transfer, spheroplast fusion and so on. Numerous techniques are known in the art for the introduction of foreign sequences into cells and may be used in accordance with the present invention, provided that the necessary developmental and physiological functions of the recipient cells are not disrupted. The technique can provide for the stable transfer of the polynucleotide to the cell, so that the polynucleotide is expressible by the cell and preferably heritable and expressible by its cell progeny. In some embodiments, the polynucleotide is introduced into the cell through transfection. Methods for transfecting erythrocytes are known in the art. See, Bacchetti and Graham (1977) Proc Natl Acad Sci USA 74(4): 1590-4; Straus and Raskas (1980) J Gen Virol 48:241-5; and Deitsch et al. (2001) Nucleic Acids Res 29(3):850-3; all of which are herein incorporated by reference. Transfection typically is carried out by mixing a cationic lipid with the material to produce liposomes which fuse with the cell plasma membrane and deposit the genetic material inside the cell.

To increase the life-span of an erythrocyte, a microRNA is introduced into the erythrocyte. As used herein, the terms “microRNA,” “miRNA,” “mature microRNA,” and “mature miRNA” refer to a single-stranded RNA molecule that is about 19 to about 25 nucleotides in length (including about 19, about 20, about 21, about 22, about 23, about 24, and about 25 nucleotides) that effectively reduces the expression level of target polynucleotides and polypeptides encoded thereby. A microRNA may be generated from various precursors, including but not limited to an endogenous primary microRNA transcript (pri-miRNA), an exogenously introduced hairpin RNA (including shRNA molecules), a transcript comprising a local hairpin structure comprising a microRNA that has been encoded by an exogenously introduced plasmid DNA, or an exogenously introduced siRNA that comprises a microRNA sequence.

Endogenous miRNAs are generated through a series of steps beginning with the transcription of a primary miRNA transcript (pri-miRNA). The pri-miRNA transcript is a single-stranded RNA molecule that comprises at least one stem-loop structure. The pri-miRNA transcript is typically thousands of nucleotides long and is often capped, spliced, and poly-adenylated and can be polycistronic, comprising multiple microRNAs of the same or different sequence.

A “stem-loop structure” refers to a polynucleotide having a secondary structure that includes a region of nucleotides which are known or predicted to form a double stranded portion (the stem portion or stem region) that is linked on one side by a region of predominantly single-stranded nucleotides (loop portion or loop region). The term “hairpin” structures are also used herein to refer to stem-loop structures. The stem-loop structures of pri-miRNA and precursor miRNAs (pre-miRNAs) comprise the microRNA sequence, which is complementary (fully or partially) to the target polynucleotide, which base pairs with the microRNA* sequence (also referred to as the “star strand”). The microRNA and microRNA* sequences make up the stem of the stem-loop structure and can be fully or partially complementary to one another.

Primary microRNA transcripts can also be encoded by exogenously introduced plasmid DNA comprising the coding sequence for the primary microRNA transcript operably linked to regulatory sequences that regulate the expression of the transcript.

Alternatively, primary microRNA transcripts can also refer to exogenously introduced hairpin-comprising single-stranded RNAs comprising a microRNA sequence that can be recognized and cleaved by the enzyme Drosha to generate a pre-miRNA.

The primary microRNA transcript is cleaved by the RNase III enzyme Drosha to release the stem-loop structure comprising the microRNA, which is now referred to as the precursor miRNA or pre-miRNA. Thus, the terms “precursor-microRNA,” “pre-miRNA,” and “precursor-miRNA” refer to a nucleic acid having a stem-loop structure with a microRNA sequence incorporated therein. Pre-miRNAs are exported from the nuclear compartment into the cytoplasm by exportin-5, where the pre-miRNAs are further processed into miRNA duplexes by the cytoplasmic RNase III Dicer. The resultant miRNA duplexes (double-stranded RNA) comprise the mature microRNA, which is the strand that will bind to the complementary (fully or partially) target polynucleotide, and the microRNA* strand or “star strand,” which is complementary to the microRNA itself (fully or partially). The mature miRNA is incorporated into RNA-induced silencing complexes (RISC) and guides the RISC to complementary RNA molecules, wherein the RISC either nucleolytically degrades the target messenger RNA (mRNA) or blocks the translation of the target mRNA, thereby inhibiting the expression. Thus, methods that employ the transfection of a miRNA or administration of a miRNA encompass the transfection or administration of any form of a miRNA, including a primary miRNA transcript, a precursor miRNA, an shRNA, an siRNA, or a microRNA itself (single-stranded) or an expression vector that encodes any one of these molecules.

A miRNA can be generated through the processing of exogenously introduced shRNAs or siRNA precursors. The terms “short hairpin RNA” or “shRNA” refer to an RNA molecule comprising at least two complementary portions hybridized or capable of hybridizing to form a double-stranded (duplex) structure sufficiently long to mediate RNAi (generally between approximately 17 and 29 nucleotides in length, including 17, 18, 19, 20, 21, 22, 23, 24, 25, 26, 27, 28, and 29 nucleotides in length, and in some embodiments, typically at least 19 base pairs in length), and at least one single-stranded portion, typically between approximately 1 and 20 or 1 to 10 nucleotides in length that forms a loop connecting the two nucleotides that form the base pair at one end of the duplex portion. The duplex portion can, but does not require, one or more bulges consisting of one or more unpaired nucleotides. In specific embodiments, the shRNAs comprise a 3′ overhang. Thus, shRNAs are precursors of siRNAs and are, in general, similarly capable of inhibiting the levels or translation of a target transcript.

A “short interfering RNA” or “siRNA” comprises an RNA duplex (double-stranded region) and can further comprise one or two single-stranded overhangs, e.g., 3′ or 5′ overhangs. The duplex can be approximately 19 base pairs (bp) long, although lengths between 17 and 29 nucleotides, including 17, 18, 19, 20, 21, 22, 23, 24, 25, 26, 27, 28, and 29 nucleotides, can be used. An siRNA can be formed from two RNA molecules that hybridize together or can alternatively be generated from a single RNA molecule that includes a self-hybridizing portion. The duplex portion of an siRNA can include one or more bulges containing one or more unpaired and/or mismatched nucleotides in one or both strands of the duplex or can contain one or more noncomplementary nucleotide pairs. Thus, the siRNA can mimic the miRNA duplex that is formed following processing by Dicer.

By “target polynucleotide” or “target mRNA” is intended the polynucleotide or mRNA that comprises a complementary sequence (fully or partially) with a microRNA, the expression of which is reduced by the complementary miRNA. The target polynucleotide or mRNA can comprise more than one region that is complementary to a particular microRNA.

The term “expression” has its meaning as understood in the art and refers to the process of converting genetic information encoded in a gene or a coding sequence into RNA (e.g., mRNA, rRNA, tRNA, or snRNA) through “transcription” of a polynucleotide (e.g., via the enzymatic action of an RNA polymerase), and for polypeptide-encoding polynucleotides, into a polypeptide through “translation” of mRNA. Thus, an “expression product” is, in general, an RNA transcribed from the gene (e.g., either pre- or post-processing) or polynucleotide or a polypeptide encoded by an RNA transcribed from the gene (e.g., either pre- or post-modification).

By “reduces” or “reducing” the expression level of a polynucleotide or a polypeptide encoded thereby is intended to mean, the level of the polynucleotide or the encoded polypeptide is statistically lower than the target polynucleotide level or encoded polypeptide level in an appropriate control which is not exposed to the microRNA. In particular embodiments, reducing the target polynucleotide level and/or the encoded polypeptide level according to the presently disclosed subject matter results in less than 95%, less than 90%, less than 80%, less than 70%, less than 60%, less than 50%, less than 40%, less than 30%, less than 20%, less than 10%, or less than 5% of the target polynucleotide level, or the level of the polypeptide encoded thereby in an appropriate control. Methods to assay for the level of the RNA transcript, the level of the encoded polypeptide, or the activity of the polynucleotide or polypeptide are discussed elsewhere herein.

In general, miRNAs reduce the expression of a target polynucleotide by directing the nucleolytic degradation of the target mRNA or inhibition of the translation of the target mRNA via the microRNA-directed RNA-induced silencing complexes (RISC). The choice of mRNA degradation or translational inhibition is dictated somewhat by the complementarity between the microRNA and the complementary region of the target mRNA. For those target mRNAs that comprise region(s) that are fully complementary to a microRNA, the target mRNA will be degraded by the microRNA-directed RISC. On the other hand, for those target mRNAs that comprise region(s) that are only partially complementary to a microRNA, the translation of the target mRNA will be inhibited by RISC. In animals, the translational inhibition of target mRNAs is the preferred mechanism of microRNAs, whereas most microRNA-targeted mRNAs are degraded in plants.

The term “complementary” is used herein in accordance with its art-accepted meaning to refer to the capacity for precise pairing via hydrogen bonds (e.g., Watson-Crick base pairing or Hoogsteen base pairing) between two nucleosides, nucleotides or nucleic acids, and the like. For example, if a nucleotide at a certain position of a first nucleic acid is capable of stably hydrogen bonding with a nucleotide located opposite to that nucleotide in a second nucleic acid, when the nucleic acids are aligned in opposite 5′ to 3′ orientation (i.e., in anti-parallel orientation), then the nucleic acids are considered to be complementary at that position (where position may be defined relative to either end of either nucleic acid, generally with respect to a 5′ end). The nucleotides located opposite one another can be referred to as a “base pair.” A complementary base pair contains two complementary nucleotides, e.g., A and U, A and T, G and C, and the like, whereas a noncomplementary base pair contains two noncomplementary nucleotides (also referred to as a mismatch). Two polynucleotides are said to be complementary to each other when a sufficient number of corresponding positions in each molecule are occupied by nucleotides that hydrogen bond with each other, i.e., a sufficient number of base pairs are complementary.

Two polynucleotide sequences are said to be fully complementary to one another when all the nucleotides within one sequence base pair with all the nucleotides in the complementary sequence. Alternatively, when there are one or more mismatches within the two complementary sequences, the two sequences are considered to be only partially complementary to one another.

The microRNA can be complementary to any region of the target mRNA to effect degradation or translational inhibition. In certain embodiments, the microRNA is complementary to the 3′ untranslated region (3′ UTR) of target mRNAs. In some embodiments, the microRNA is only partially complementary to the target mRNA. In some of these embodiments, only nucleotides 2-8 (counting from the 5′ end) of the miRNA is complementary to the target mRNA. A miRNA or miRNA precursor can share at least about 80%, 85%, 90%, 91%. 92%, 93%, 94%, 95%, 96%, 97%, 98%, 99%, or 100% sequence complementarity with the target transcript for a stretch of at least about 15, 16, 17, 18, 19, 20, 21, 22, 23, 24, 25, 26, 27, 28, 29, or 30 nucleotides. In specific embodiments, the region of precise sequence complementarity is interrupted by a bulge. See, Ruvkun (2001) Science 294: 797-799, Zeng et al. (2002) Molecular Cell 9:1-20, and Mourelatos et al. (2002) Genes Dev 16:720-728.

There is also evidence that in plants, and possibly in animals, in addition to post-transcriptional silencing mechanisms, miRNAs also might direct transcriptional silencing of the genes that encode miRNA target mRNAs through methylation of the gene. In some circumstances, the gene encoding the target mRNA (target gene) is transcriptionally silenced by microRNA-directed chromatin methylation of the target gene (Bao et al. (2004) Dev. Cell 7:653-662; Mette et al. (2000) EMBO J. 19:5194-5201; Hamilton et al. (2002) EMBO J 21:4671-4679; Zilberman et al. (2003) Science 299:716-719).

The ability of a microRNA to reduce the level of the target polynucleotide can be assessed directly by measuring the amount of the target transcript using, for example, Northern blots, nuclease protection assays, reverse transcription (RT)-PCR, real-time RT-PCR, microarray analysis, and the like. Alternatively, the ability of the miRNA to reduce the level or inhibit the translation of the target polynucleotide can be measured directly using a variety of affinity-based approaches (e.g., using a ligand or antibody that specifically binds to the target polypeptide) including, but not limited to, Western blots, immunoassays, ELISA, flow cytometry, protein microarrays, and the like. In still other methods, the ability of the miRNA to reduce the level or inhibit the translation of the target polynucleotide can be assessed by measuring a functional activity of the polypeptide encoded by the transcript or by measuring a signal produced by the polypeptide encoded by the transcript.

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

1. Expression Vectors and Host Cells

As discussed above, the miRNA employed in the methods of the invention can comprise a DNA molecule which when transcribed produces a miRNA or a precursor thereof (e.g., primary transcript, precursor miRNA, miRNA duplex). In such embodiments, the DNA molecule encoding the miRNA or precursor thereof is found in an expression cassette.

The expression cassette comprises one or more regulatory sequences, selected on the basis of the cells to be used for expression, operably linked to a polynucleotide encoding the miRNA or precursor thereof. “Operably linked” is intended to mean that the nucleotide sequence of interest (i.e., a DNA encoding a microRNA or precursor thereof) is linked to the regulatory sequence(s) in a manner that allows for expression of the nucleotide sequence (e.g., in an in vitro transcription/translation system or in a cell when the expression cassette or vector is introduced into a cell). “Regulatory sequences” include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cells and those that direct expression of the nucleotide sequence only in certain host cells (e.g., tissue-specific regulatory sequences). It will be appreciated by those skilled in the art that the design of the expression cassette can depend on such factors as the choice of the host cell to be transformed, the level of expression of the miRNA or precursor thereof, and the like. Such expression cassettes typically include one or more appropriately positioned sites for restriction enzymes, to facilitate introduction of the nucleic acid into a vector.

It will further be appreciated that appropriate promoter and/or regulatory elements can readily be selected to allow expression of the relevant miRNA or precursor thereof in the cell of interest. In certain embodiments, the promoter utilized to direct intracellular expression of a miRNA or precursor thereof is a promoter for RNA polymerase III (Pol III). References discussing various Pol III promoters, include, for example, Yu et al. (2002) Proc. Natl. Acad. Sci. 99(9), 6047-6052; Sui et al. (2002) Proc. Natl. Acad. Sci. 99(8), 5515-5520 (2002); Paddison et al. (2002) Genes and Dev. 16, 948-958; Brummelkamp et al. (2002) Science 296, 550-553; Miyagashi (2002) Biotech. 20, 497-500; Paul et al. (2002) Nat. Biotech. 20, 505-508; Tuschl et al. (2002) Nat. Biotech. 20, 446-448. According to other embodiments, a promoter for RNA polymerase I, e.g., a tRNA promoter, can be used. See McCown et al. (2003) Virology 313(2):514-24; Kawasaki (2003) Nucleic Acids Res. 31 (2):700-7.

The regulatory sequences can also be provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2d ed., Cold Spring Harbor Laboratory Press, Plainview, N.Y.). See, Goeddel (1990) in Gene Expression Technology: Methods in Enzymology 185 (Academic Press, San Diego, Calif.).

In vitro transcription can be performed using a variety of available systems including the T7, SP6, and T3 promoter/polymerase systems (e.g., those available commercially from Promega, Clontech, New England Biolabs, and the like). Vectors including the T7, SP6, or T3 promoter are well known in the art and can readily be modified to direct transcription of miRNAs or precursors thereof. When double-stranded miRNA precursors are synthesized in vitro, the strands can be allowed to hybridize before introducing into a cell or before administration to a subject. As noted above, miRNAs or precursors thereof can be delivered or introduced into a cell as a single RNA molecule including self-complementary portions (e.g., an shRNA that can be processed intracellularly to yield a miRNA), or as two strands hybridized to one another. In other embodiments, the miRNAs or precursors thereof are transcribed in vivo. As discussed elsewhere herein, regardless of if the miRNA or precursor thereof is transcribed in vivo or in vitro, in either scenario, a primary transcript can be produced which can then be processed (e.g., by one or more cellular enzymes) to generate the miRNA that accomplishes gene inhibition.

D. Methods for Treatment

The present invention also contemplates methods for treating a subject in need thereof by administering a microRNA or a compound that inhibits the activity of a microRNA. In some embodiments, the subject in need thereof is a subject desiring a reduced susceptibility to malaria. In certain embodiments, the subject has an erythrocyte disease, including but not limited to a sickle cell disease, anemia, or malaria.

As used herein, the terms “treatment” or “prevention” refer to obtaining a desired pharmacologic and/or physiologic effect. The effect may be prophylactic in terms of completely or partially preventing a particular infection or disease or sign or symptom thereof and/or may be therapeutic in terms of a partial or complete cure of an infection or disease and/or adverse effect attributable to the infection or the disease. Accordingly, the method “prevents” (i.e., delays or inhibits) and/or “reduces” (i.e., decreases, slows, or ameliorates) the detrimental effects of a disease or disorder in the subject receiving the compositions of the invention.

In some embodiments, a subject with anemia or with a sickle cell disease (SCD) is treated via administration of a polynucleotide comprising or encoding a microRNA or a precursor thereof, wherein the microRNA comprises miR-320. As presented herein, miR-320 is expressed at low levels in HbSS red blood cells and is responsible for downregulating the CD71 differentiation marker. Furthermore, inhibition of miR-320 results in a reduction in cell survival. Thus, while not bound by any particular theory or mechanism, overexpression or introduction of miR-320 can reverse the dysregulated differentiation state of SCD erythrocytes and can enhance survival of erythrocytes or enhance the differentiation of erythrocytes. The miR-320 microRNA has the sequence set forth in SEQ ID NO: 10. Thus, methods for treating a subject with anemia or sickle cell disease comprise administering to said subject a polynucleotide comprising or encoding a miRNA or a precursor thereof, wherein said miRNA has a nucleotide sequence having at least 80%, at least 85%, at least 90%, at least 95%, at least 96%, at least 97%, at least 98%, at least 99%, or 100% of the sequence set forth in SEQ ID NO: 10, wherein the polynucleotide when expressed or introduced into a red blood cell enhances the survival of the red blood cell. In some embodiments, the miRNA or precursor thereof comprises the first 8 nucleotides of the sequence set forth in SEQ ID NO: 10.

As noted herein, transfection of an erythrocyte with a microRNA, including but not limited miR-181a, Let-7a-1, and miR-17, can increase the life-span of an erythrocyte. Thus, microRNAs, such as miR-181a, Let-7a-1, and miR-17 can be administered to a subject with anemia or a sickle cell disease to treat the disease. In some of these embodiments, a subject with anemia or sickle cell disease is administered a polynucleotide comprising or encoding a miRNA or a precursor thereof, wherein said miRNA has a nucleotide sequence having at least 80%, at least 85%, at least 90%, at least 95%, at least 96%, at least 97%, at least 98%, at least 99%, or 100% of the sequence set forth in SEQ ID NO: 7, 8, or 9, wherein the polynucleotide when expressed or introduced into an erythrocyte increases the life-span of the erythrocyte. In some embodiments, the miRNA or precursor thereof comprises the first 8 nucleotides of the sequence set forth in SEQ ID NO: 7, 8, or 9.

In other embodiments, a subject with anemia or a sickle cell disease is administered a compound that inhibits the activity of a miR-144 or a miR-142-5p microRNA. The sequences of the miR-144 and miR-142-5p microRNAs are set forth in SEQ ID NO: 11 and 12, respectively. Data provided herein demonstrate that miR-144 and miR-142-5p are expressed at relatively high levels in subtypes of SCD associated with high levels of hemolysis and overexpression of either microRNA led to a significantly decreased MCV and increased MCHC. Without being bound by any theory or mechanism of action, the decreased MCV and increased MCHC in the presence of miR-144 and miR-142-5p could contribute to increased HbS polymerization and sickling of red blood cells. Thus, the administration of a compound that inhibits the activity of miR-144 or miR-142-5p microRNA is useful for the treatment of anemia and sickle cell disease.

The phrase “activity of a miRNA” refers to the ability of a miRNA to reduce the expression of a target polynucleotide or a biological function that has been associated with the miRNA.

Non-limiting examples of such a compound include small molecule inhibitors and polynucleotides. Polynucleotides comprising a sequence that is complementary to the microRNA that is to be inhibited can be administered to a patient. Such polynucleotides will hybridize with the microRNA and inhibit its ability to hybridize with its target polynucleotide, thus preventing the reduction in the expression of the target polynucleotide. These polynucleotides may comprise either DNA or RNA. In some embodiments, a polynucleotide comprising or encoding a nucleic acid sequence that is complementary to a miRNA, wherein said miRNA has a nucleotide sequence having at least 80%, at least 85%, at least 90%, at least 95%, at least 96%, at least 97%, at least 98%, at least 99%, or 100% sequence identity to SEQ ID NO: 11 can be used to inhibit the activity of miR-144. In other embodiments, a polynucleotide comprising or encoding a nucleic acid sequence that is complementary to a miRNA, wherein said miRNA has a nucleotide sequence having at least 80%, at least 85%, at least 90%, at least 95%, at least 96%, at least 97%, at least 98%, at least 99%, or at least 100% sequence identity to SEQ ID NO: 12 can be used to inhibit the activity of miR-142-5p. In some of these embodiments, the polynucleotide is single-stranded. In some embodiments, the miRNA or precursor thereof comprises the first 8 nucleotides of the sequence set forth in SEQ ID NO: 11 or 12.

In some embodiments, the polynucleotide inhibitor comprises a modified nucleotide (deoxyribonucleotide or ribonucleotide) that imparts stability and provides protection from degradation by nucleases. Such modified deoxyribonucleotide moieties comprise, for example, phosphorothioate deoxyribose groups as the backbone unit (Eckstein (200) Antisense Nucleic Acids Drug Dev. 10: 117-121) or an N′3-N′5 phosphoroamidate deoxyribonucleotide moiety, which comprises an N′3-N′5 phosphoroamidate deoxyribose group as the backbone unit (Gryaznov et al. (1994) J. Am. Chem. Soc. 116: 3143-3144).

In other embodiments, the polynucleotide comprises at least one modified ribonucleotide moiety. Non-limiting examples of modified ribonucleotide moieties include a ribonucleotide moiety that is substituted at the 2′ position, such as with an alkyl or alkyloxy group (e.g., 2′-O-methoxyethyl RNA) or a fluoro group (Damha et al. (1998) J. Am. Chem. Soc. 120: 12976-12977). The polynucleotide also may be a locked nucleic acid molecule, which comprises a ribonucleotide that has a methylene bridge between the 2′-oxygen atom and the 4′-carbon atom. See, for example, Kurreck et al. (2002) Nucleic Acids Res. 30: 1911-1918; Elayadi et al. (2001) Curr. Opinion Invest. Drugs 2: 558-561; Orum et al. (2001) Curr. Opinion Mol. Ther. 3: 239-243; Koshkin et al. (1998) Tetrahedron 54: 3607-3630; Obika et al. (1998) Tetrahedron Lett. 39: 5401-5404. In other embodiments, the polynucleotide comprises peptide nucleic acid (PNA) moieties that comprise a base bonded to an amino acid residue as the backbone unit (Nielson (1999) Methods Enzymol. 313: 156-164; Elayadi, et al. (2001) Curr. Opinion Invest. Drugs 2: 558-561; Braasch et al.(2002) Biochemistry 41: 4503-4509, Nielsen et al. (1991) Science 254: 1497-1500). In other non-limiting examples of a modified polynucleotide, the molecule comprises at least one morpholino phosphoroamidate nucleotide moiety (Heasman (2002) Dev. Biol. 243: 209-214), at least one cyclohexene nucleotide moiety (Wang et al. (2000) J. Am. Chem. Soc. 122: 8595-8602, Verbeure et al. (2001) Nucleic Acids Res. 29: 4941-4947), or at least one tricyclo nucleotide moiety (Steffens et al. (1997) J. Am. Chem. Soc. 119: 11548-11549, Renneberg et al. (2002) J. Am. Chem. Soc. 124: 5993-6002.

The present invention also provides methods for the treatment of subjects with malaria, said method comprising administering to the subject a microRNA that when expressed by or introduced into a red blood cell is capable of being translocated into a malaria parasite (e.g., Plasmodium falciparum) infecting the red blood cell and inhibiting the growth or survival of the parasite. In some embodiments, the subject with malaria is administered a polynucleotide comprising or encoding a miRNA or a precursor thereof, wherein said miRNA has a nucleotide sequence having at least 80%, at least 85%, at least 90%, at least 95%, at least 96%, at least 97%, at least 98%, at least 99%, or 100% sequence identity to SEQ ID NO: 13 or 14, wherein the miRNA has the ability to be translocated into a malaria parasite infecting a red blood cell and to inhibit the growth or survival of said malaria parasite. The sequences of the miR-451 and miR-223 microRNAs are set forth in SEQ ID NO: 13 and 14, respectively. In some embodiments, the miRNA or precursor thereof comprises the first 8 nucleotides of the sequence set forth in SEQ ID NO: 13 or 14, wherein the miRNA has the ability to be translocated into a malaria parasite infecting a red blood cell and to inhibit the growth or survival of said malaria parasite.

Methods are also provided for reducing the susceptibility of a subject to malaria, said method comprising administering to the subject a microRNA that when expressed by or introduced into a red blood cell is capable of being translocated into a malaria parasite (e.g., Plasmodium falciparum) infecting the red blood cell and inhibiting the growth or survival of the parasite. In some embodiments, the subject with malaria is administered a polynucleotide comprising or encoding a miRNA or a precursor thereof, wherein said miRNA has a nucleotide sequence having at least 80%, at least 85%, at least 90%, at least 95%, at least 96%, at least 97%, at least 98%, at least 99%, or at least 100% sequence identity to SEQ ID NO: 13 or 14, wherein said miRNA has the ability to be translocated into a malaria parasite infecting a red blood cell and to inhibit the growth or survival of said malaria parasite. In some embodiments, the miRNA or precursor thereof comprises the first 8 nucleotides of the sequence set forth in SEQ ID NO: 13 or 14, wherein the miRNA has the ability to be translocated into a malaria parasite infecting a red blood cell and to inhibit the growth or survival of said malaria parasite

Delivery of a therapeutically effective amount of a polynucleotide or a compound that inhibits the activity of a microRNA can be obtained via administration of a pharmaceutical composition comprising a therapeutically effective dose of this agent. By “therapeutically effective amount” or “dose” is meant the concentration of a microRNA that is sufficient to elicit the desired therapeutic effect.

As used herein, “effective amount” is an amount sufficient to effect beneficial or desired clinical or biochemical results. An effective amount can be administered one or more times.

The effective amount of the polynucleotide or compound will vary according to the weight, sex, age, and medical history of the subject. Other factors which influence the effective amount can include, but are not limited to, the severity of the subject's condition, the disorder being treated, the stability of the polynucleotide or compound, and, if desired, the adjuvant therapeutic agent being administered along with the polynucleotide. Methods to determine efficacy and dosage are known to those skilled in the art. See, for example, Isselbacher et al. (1996) Harrison's Principles of Internal Medicine 13 ed., 1814-1882, herein incorporated by reference.

Toxicity and therapeutic efficacy of such compounds can be determined by standard pharmaceutical in cell cultures or experimental animals, e.g., for determining the LD₅₀ (the dose lethal to 50% of the population) and the ED₅₀ (the dose therapeutically effective in 50% of the population).

The dose ratio between toxic (e.g., immunotoxic) and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the presently disclosed methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical formulation typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical formulation can be administered at various intervals and over different periods of time as required, e.g., multiple times per day, daily, every other day, once a week for between about 1 to 10 weeks, between 2 to 8 weeks, between about 3 to 7 weeks, about 4, 5, or 6 weeks, and the like. The skilled artisan will appreciate that certain factors can influence the dosage and timing required to effectively treat a subject, including but not limited to the severity of the disease, disorder, or unwanted condition, previous treatments, the general health and/or age of the subject, and other diseases or unwanted conditions present. Generally, treatment of a subject can include a single treatment or, in many cases, can include a series of treatments. Further, treatment of a subject can include a single cosmetic application or, in some embodiments, can include a series of cosmetic applications.

Exemplary doses include milligram or microgram amounts of the inventive compound per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) For local administration (e.g., intranasal), smaller doses can be used. It is furthermore understood that appropriate doses of a compound depend upon its potency and can optionally be tailored to the particular recipient, for example, through administration of increasing doses until a preselected desired response is achieved. It is understood that the specific dose level for any particular animal subject can depend on a variety of factors including the activity of the specific compound employed, the age, body weight, general health, gender, and diet of the subject, the time of administration, the route of administration, the rate of excretion, any drug combination, and the degree of expression or activity to be modulated.

One of ordinary skill in the art upon review of the presently disclosed subject matter would appreciate that the presently disclosed compounds, including pharmaceutical compositions thereof, can be administered directly to a cell, a cell culture, a cell culture medium, a tissue, a tissue culture, a tissue culture medium, and the like. When referring to the compounds of the invention, the term “administering,” and derivations thereof, comprises any method that allows for the compound to contact a cell. The presently disclosed compounds can be administered to (or contacted with) a cell or a tissue in vitro or ex vivo. The presently disclosed compounds also can be administered to (or contacted with) a cell or a tissue in vivo by administration to an individual subject, e.g., a patient, for example, by systemic administration (e.g., intravenous, intraperitoneal, intramuscular, subdermal, or intracranial administration) or topical application, as described elsewhere herein.

E. Methods for the Identification of Additional Erythrocyte miRNAs Associated with Erythrocyte Diseases

The present invention also provides methods for the identification of additional miRNAs that are associated with or can distinguish subtypes of an erythrocyte disease. In particular, the methods include the identification of erythrocyte miRNAs that distinguish sickle-cell disease subtypes and other anemia disorders. These methods include the identification of erythrocyte miRNAs which predict the phenotypic subtypes of HbSS patients; the identification of erythrocyte miRNAs which distinguish patients with HbSS, sickle cell trait (HbSA), and other hemolytic conditions not caused by HbS; and the identification of anemia-specific erythrocyte miRNAs. Such methods are described in Experimental Examples 3 and 4. In general, the methods involve obtaining erythrocytes from a population of subjects, wherein said population comprises subjects having said erythrocyte disease, determining the composition of miRNA present in said erythrocytes from each subject within the population, and performing an analysis (e.g., supervised or unsupervised) of the miRNA composition from each subject within the population to identify an erythrocyte miRNA that is predictive of the susceptibility to or severity of an erythrocyte disease, is indicative of the presence of the erythrocyte disease. or that distinguishes subtypes of the erythrocyte disease. The population of subjects can comprise subjects with varying severity of an erythrocyte disease or with different subtypes of the erythrocyte disease to allow for the identification of those miRNAs that can predict the severity of a disease or distinguish subtypes of a disease. In some embodiments, the population of subjects further comprises control subjects, such as healthy subjects who are not known to have or are not suspected of having the disease.

The analysis of the miRNA composition from each subject within the population can be performed using any form of data analysis known in the art, including those methods outlined elsewhere herein (see Experimental Examples 1 and 3). Generally, the miRNA expression data from each subject within the population will be analyzed together to provide the expression profile of miRNAs across the population of subjects.

In some embodiments, the methods further comprise analyzing the function of the miRNA or set of miRNAs in the development or progression of the erythrocyte disease. These methods can include overexpressing the miRNA within an erythrocyte or treating an erythrocyte with a compound that inhibits the activity of the miRNA (such as a polynucleotide with a sequence that is complementary to the miRNA) and assaying the effect.

### II. Compositions

The present invention provides pharmaceutical compositions that can be used for the treatment of an erythrocyte disease and kits for the detection of the levels of particular miRNAs.

1. Pharmaceutical Compositions

The polynucleotides and compounds described herein are useful in mammalian tissue culture systems, in animal studies, and for therapeutic purposes. The presently disclosed compositions can be formulated for delivery, i.e., administering to the subject, by any available route including, but not limited, to parenteral (e.g., intravenous), intradermal, subcutaneous, oral, nasal, bronchial, opthalmic, transdermal (topical), transmucosal, rectal, and vaginal routes. In some embodiments, the route of delivery is intravenous, parenteral, transmucosal, nasal, bronchial, vaginal, and oral.

The presently disclosed pharmaceutical compositions also can include a polynucleotide or compound with a pharmaceutically acceptable carrier. As used herein the term “pharmaceutically acceptable carrier” includes solvents, dispersion media, coatings, antibacterial and antifungal agents, isotonic and absorption delaying agents, and the like, compatible with pharmaceutical administration. Supplementary active compounds also can be incorporated into the compositions.

Pharmaceutical composition for the treatment of an erythrocyte disease include those comprising a pharmaceutical carrier and a composition selected from the group consisting of:
a) a polynucleotide comprising or encoding a miRNA or a precursor thereof, wherein said miRNA has a nucleotide sequence having at least 90% sequence identity to SEQ ID NO: 10, and wherein said polynucleotide when expressed or introduced into a red blood cell enhances the survival of said red blood cell;
b) a polynucleotide comprising or encoding a miRNA or a precursor thereof, wherein said miRNA has a nucleotide sequence having at least 90% sequence identity to SEQ ID NO: 7, 8 or 9, and wherein said polynucleotide when expressed or introduced into an erythrocyte increases the life-span of said erythrocyte;
c) a polynucleotide comprising or encoding a miRNA or a precursor thereof, wherein said miRNA has a nucleotide sequence having at least 90% sequence identity to SEQ ID NO: 13 or 14, and wherein said miRNA has the ability to be translocated into a malaria parasite infecting a red blood cell and to inhibit the growth or survival of said malaria parasite; and
d) a compound that inhibits the activity of at least one of miR-144 and miR-142-5p.

In some embodiments, the compound that inhibits the activity of at least one of miR-144 and miR-142-5 comprises a polynucleotide comprising or encoding a nucleotide sequence that is complementary to a miRNA, wherein said miRNA has a nucleotide sequence having at least 90% sequence identity to SEQ ID NO: 11 or 12.

As one of ordinary skill in the art would appreciate, a presently disclosed pharmaceutical composition is formulated to be compatible with its intended route of administration. Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates; and agents for the adjustment of tonicity, such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The composition should be sterile and should be fluid to the extent that easy syringability exists. In some embodiments, the pharmaceutical compositions are stable under the conditions of manufacture and storage and should be preserved against the contaminating action of microorganisms, such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, isotonic agents, for example, sugars, polyalcohols, such as manitol or sorbitol, or sodium chloride are included in the formulation. Prolonged absorption of the injectable formulation can be brought about by including in the formulation an agent that delays absorption, for example, aluminum monostearate and gelatin.

Sterile injectable solutions can be prepared by incorporating the active compound (e.g., polynucleotide) in the required amount in an appropriate solvent with one or a combination of ingredients enumerated above, as required, followed by filtered sterilization. In certain embodiments, solutions for injection are free of endotoxin. Generally, dispersions are prepared by incorporating the active compound into a sterile vehicle which contains a basic dispersion medium and the required other ingredients from those enumerated above. In those embodiments in which sterile powders are used for the preparation of sterile injectable solutions, the solutions can be prepared by vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions also can be prepared using a fluid carrier for use as a mouthwash. Pharmaceutically or cosmetically compatible binding agents, and/or adjuvant materials can be included as part of the composition. The tablets, pills, capsules, troches, and the like can contain any of the following ingredients, or compounds of a similar nature: a binder, such as microcrystalline cellulose, gum tragacanth or gelatin; an excipient, such as starch or lactose, a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring. Compositions for oral delivery can advantageously incorporate agents to improve stability within the gastrointestinal tract and/or to enhance absorption.

For administration by inhalation, the presently disclosed compositions can be delivered in the form of an aerosol spray from a pressured container or dispenser which contains a suitable propellant, e.g., a gas such as carbon dioxide, or a nebulizer. Liquid aerosols, dry powders, and the like, also can be used.

Systemic administration of the presently disclosed compositions also can be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds also can be prepared in the form of suppositories (e.g., with conventional suppository bases such as cocoa butter and other glycerides) or retention enemas for rectal delivery.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical or cosmetic carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of individuals. Guidance regarding dosing is provided elsewhere herein.

Depending on the route of administration, the agent may be coated in a material to protect it from the action of enzymes, acids and other natural conditions which may inactivate the agent. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

To administer an agent by other than parenteral administration, it may be necessary to coat the agent with, or co-administer the agent with, a material to prevent its inactivation. Enzyme inhibitors include pancreatic trypsin inhibitor, diisopropylfluorophosphate (DEP) and trasylol. Liposomes include water-in-oil-in-water emulsions as well as conventional liposomes (Strejan et al. (1984) J. Neuroimmunol 7:27).

When administering a polynucleotide, the polynucleotide can be injected directly as naked DNA or RNA, by infection using defective or attenuated retrovirals or other viral vectors, or can be coated with lipids or cell-surface receptors or transfecting agents, encapsulated in liposomes, microparticles, or microcapsules, or by administering them in linkage to a peptide which is known to enter the nucleus, by administering it in linkage to a ligand subject to receptor-mediated endocytosis (see, e.g., Wu and Wu (1987) J. Biol. Chem. 262:4429-4432) (which can be used to target cell types specifically expressing the receptors) and so on. In another embodiment, polynucleotide-ligand complexes can be formed in which the ligand comprises a fusogenic viral peptide to disrupt endosomes, allowing the polynucleotide to avoid lysosomal degradation. In yet another embodiment, the polynucleotide can be targeted in vivo for cell specific uptake and expression, by targeting a specific receptor. Alternatively, the polynucleotide can be introduced intracellularly and incorporated within host cell DNA for expression, by homologous recombination (Koller and Smithies (1989) Proc. Natl. Acad. Sci. USA 86:8932-8935; Zijistra et al. (1989) Nature 342:435-438).

2. Kits

The present invention also provides kits useful for determining the susceptibility of a subject to an erythrocyte disease, for determining the severity of an erythrocyte disease, or for monitoring the progression of an erythrocyte disease. These kits comprise reagents (e.g., primers) sufficient for the detection of at least one of miR-144, miR-142-5p, miR-451, miR-223, miR-320, miR-23b, and miR-221, miR-181a, Let-7a-1, miR-17, or any miRNA listed in Table 1.

In another embodiment, the kit comprises a set of oligonucleotide primers sufficient for the detection and/or quantitation of at least one of the miRNAs listed above. The oligonucleotide primers may be provided in a lyophilized or reconstituted form, or may be provided as a set of nucleotide sequences. In one embodiment, the primers are provided in a microplate format, where each primer set occupies a well (or multiple wells, as in the case of replicates) in the microplate. The microplate may further comprise primers sufficient for the detection of one or more normalization RNAs as discussed infra. The kit may further comprise reagents and instructions sufficient for the amplification of miRNAs.

The following examples are offered by way of illustration and not by way of limitation.

### EXPERIMENTAL

### Materials and Methods for Examples 1 and 2

### Human Blood Collection, Mature Erythrocyte Purification and Cell Lines

This study was conducted with the approval of the Duke University Institutional Review Board (IRB), and informed consent from each donor was obtained. Blood was obtained by venipuncture and collected into sodium citrate-containing tubes. In the first purification scheme, leukocytes in whole blood were first removed by filtering through a PALL PurecellNeo filter (Pall Biomedical Co, NY). The cells in the filtrate were separated in a Ficoll-hypaque gradient to eliminate remaining leukocytes and platelets. The remaining erythrocytes underwent further antibody depletion with autoMAC™ following the manufacturer's instructions (MACS; Miltenyi Biotec, Auburn, Calif.) to remove CD45 (leukocyte common antigen, LCA) positive leukocytes and CD71 (transferrin receptor) positive reticulocytes with magnetic beads conjugated with respective antibodies (Miltenyi Biotec). In the second erythrocyte purification scheme, whole blood was first subject to a Ficoll-hypaque gradient to remove platelets and leukocytes (Zen et al. (2004) Am. J. Hematol. 75: 63-72). Packed erythrocytes were subjected to an arabinogalactan density gradient (Larex, St Paul, Minn.), comprising three layers: 1.085, 1.090, and 1.095 g/ml respectively, to separate reticulocytes from mature erythrocytes. Reticulocytes migrate to the low density area, whereas mature erythrocytes migrate to the high density layer and then were collected for RNA extraction. In both approaches, the purity of the purified cell population was further evaluated with FACS for surface expression of CD235a (glycophorin A), CD71 (transferrin receptor), CD16 (FcγRIII) and CD45 (LCA) with fluorescently labeled antibodies (BD Pharmingen, San Diego, Calif.). For further reticulocyte purification for RNA isolation, packed erythrocytes obtained from leukocyte-removal filtrate were incubated with CD71-MACS beads (Miltenyi Biotec). After washing with staining buffer (Hanks' balanced salt solution with 2% FCS and 0.02% NaN3), CD71+ reticulocytes were enriched with autoMACS by POSSEL_S mode, immunostained with FITC-conjugated CD71 antibodies (BD Pharmingen) and positive cells sorted by FACS. New methylene blue N solution containing potassium oxalate, and New Methylene Blue N Zinc chloride double salt was obtained from VWR. The indicated purified cells were mixed with the same volume of staining solution for 30 min, and then cytospun onto microscopic slides for examination. The K562 cell line was a gift from Dr. Murat Arcasoy and was maintained in RPMI supplemented with 10% serum, glutamine and antibiotics.

### RNA Extraction, Electrophoresis, RT-PCR Analysis, and Northern Blot Analysis.

Total RNA was extracted using the mirVana microRNA Isolation Kit (Ambion, Austin, Tex.) according to the manufacturer's protocol. For the RT-PCR assay to evaluate the cell purity, the following primers were used to amplify the indicated gene products HBB-forward: gcaacctcaaacagacacca (SEQ ID NO: 15); HBB-reverse: agctcactcagtgtggcaaa (SEQ ID NO: 16); CD45-forward: acgtaatggaagtgctgcaatg (SEQ ID NO: 17); CD45-reverse: gcatactattatctgatgtcatggagaca (SEQ ID NO: 18).

For northern blot analyses, 20 μg of total RNA were separated on a 15% TBE-urea polyacrylamide gel (Bio-Rad, Hercules, Calif.), and electro-transferred onto Hybond-N+ (Amersham Biosciences, Little Chalfont, UK) membranes. The blot was then probed with end-labeled DNA probes complementary to each indicated microRNA or U6 RNA. Hybridization was carried out in Express-hyb buffer (Clontech, Mountain View, Calif.) at 37° C.

Analysis of microRNA Expression by Microarrays.

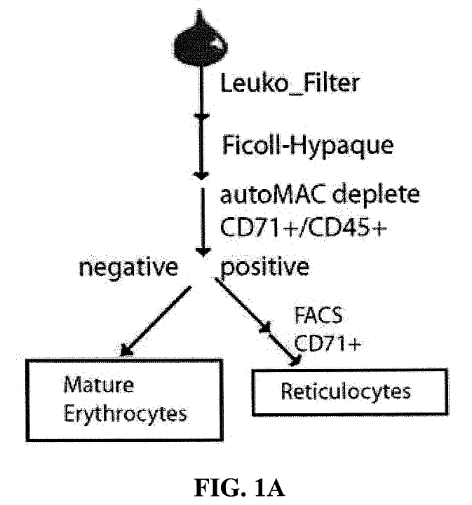

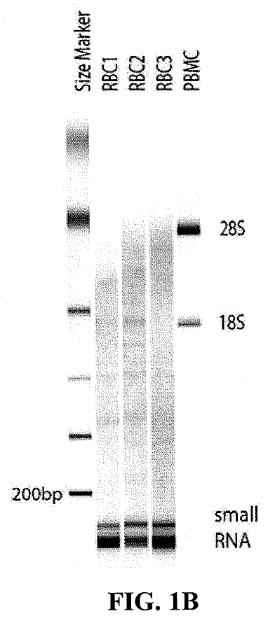

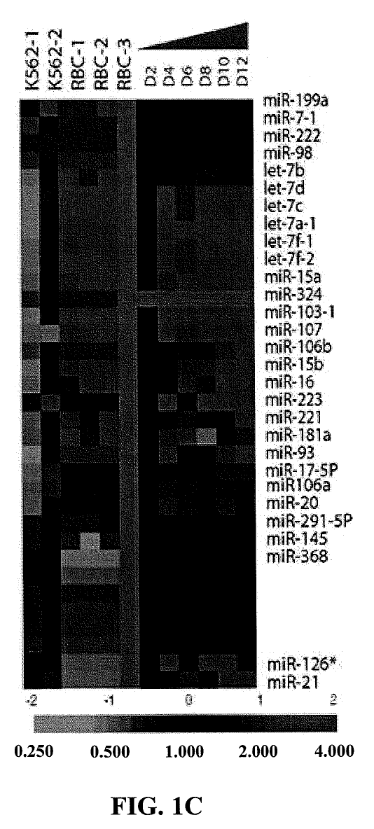

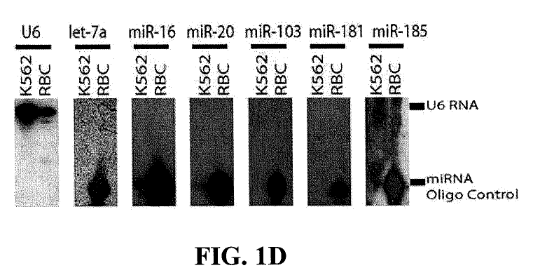

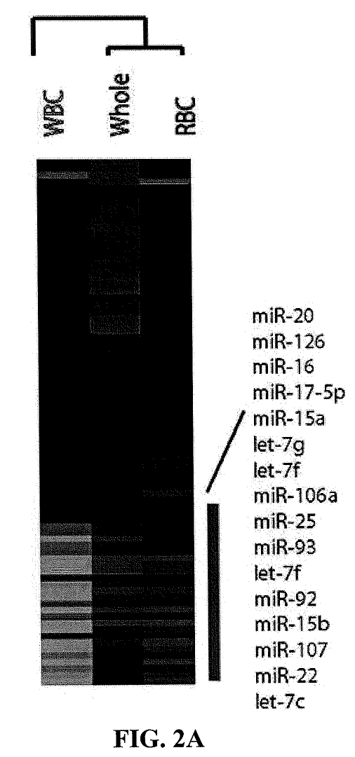

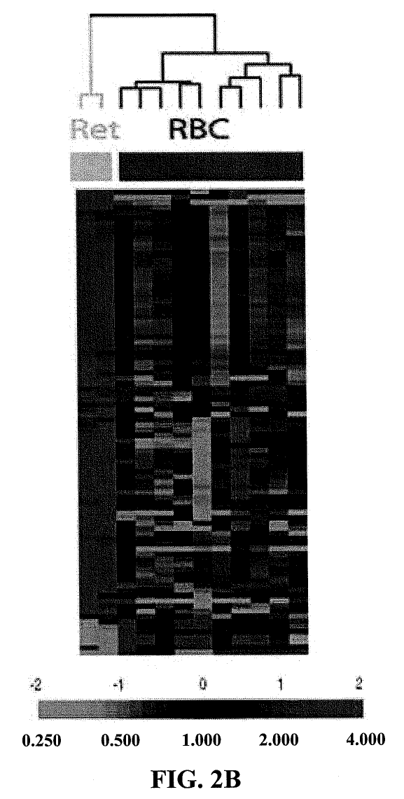

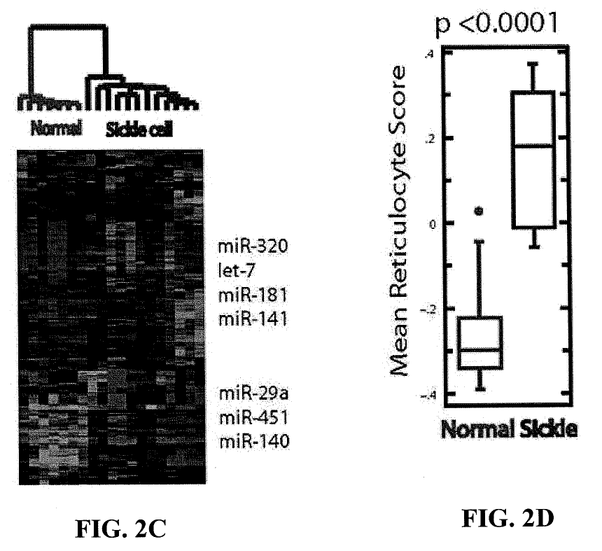

For microRNA expression profiling by microarrays in FIG. 1, 20 μg of total RNA were first size-fractionated and cleaned with the flashPAGE Fractionator (Ambion, Tex.) to collect small RNAs smaller than 40 nucleotides. 40 ng of the fractionated RNAs were then labeled with the mirVana miRNA Labeling Kit (Ambion) and amine-reactive dyes according to the manufacture's instructions. Fractionated small RNAs from erythrocyte, K562, whole blood and leukocytes were labeled with Cy5; while 293T small RNAs were labeled with Cy3 as a common reference for all samples. In experiments that compared sickle erythrocyte expression to normal individuals (FIG. 2C), 10 μg of total erythrocyte RNA per sample was labeled using the miRCURY™ LNA Array Labeling Kit (Exiqon, Vedbaek Denmark). Erythrocyte RNAs were labeled with Cy3; while whole blood RNAs were labeled as common reference with Cy5. The fluorescently labeled miRNAs were mixed with 2× hybridization buffer (Exiqon, Vedbaek Denmark) and heated at 95° C. for 3 min before hybridizing with the printed microRNA microarrays for 12-16 h in a 42° C. water bath in sealed cassettes. Following hybridization, the slides were washed and dried prior to high-resolution scan on a GenePix 4000B Array Scanner (Axon, Union City, Calif.). Each element was located and analyzed using the GenePix Pro 5.0 software package (Axon). These data were submitted into the Duke Microarray Database (DMD) for further analysis similar to the previously reported Stanford Microarray Database (Chi et al. (2006) PLoS Med. 3:e47). Data were first normalized globally per array, such that the average LogRatio was 0 after normalization. All gene elements used for analysis had an average sum intensity 2.5 fold above either channels, and a regression ratio>0.6 before further filtering based on variations among different samples. The global miRNA gene expression patterns of the indicated samples were sorted based on similarity by hierarchical clustering of gene elements that were selected from the total data set with variations to the mean expression level greater than 2 fold in at least 2 cell samples. The expression ratios of the genes were then mean-centered and arranged by hierarchical clustering using Pearson correlation (Eisen et al. (1998) Proc Natl Acad Sci USA 95; 14863-14868). The samples were also arranged by hierarchical clustering to identify their similarity of gene expression.

To obtain the average gene expression value of reticulocyte-specific miRNAs in different erythrocyte samples, we found that 56 miRNAs out of the 83 reticulocyte-specific miRNAs were represented on the miRNA microarrays. The average gene expression values of these gene elements were extracted from each examples and mean expression values were calculated and compared.

Expression Profiling of microRNA Using Multiplexing RT-PCR Assays

The 192-plex RT-PCR assays that were developed for profiling 192 individual human microRNAs were used to perform microRNA profiling with 30 ng of total RNA from each RBC or reticulocyte sample (Lao et al. (2006) Biochem. Biophys. Res. Commun. 343: 85-89). Briefly, step 1 is a multiplexed reverse transcription reaction which reverse transcribes targeted microRNAs into cDNAs in a single reaction using 192 microRNA-specific stem-loop RT primers; step 2 is a multiplexed PCR reaction with 192 sets of microRNA-specific forward primers and a universal reverse primer that amplifies the cDNA products to provide enough samples for step 3. Linear amplification was achieved with 14 PCR cycles. Step 3 is done as simultaneous, individual single-plex TaqMan® real-time PCR reactions to monitor the abundance of each microRNA after the multiplexed RT-PCRs. Real-time PCR was performed on an AB 7900 HT Sequence Detection System in a 384-well plate format, with the temperature regime consisting of a hot start at 95° C. for 10 min, followed by 40 cycles of 95° C. for 15 s, and 60° C. for 1 min. The real-time PCRs for each microRNA were run in duplicates. 189 out of 192 microRNA targets were analyzed by TaqMan assays while three assays, has-miR-16, has-miR-124a and has-miR-130b, were not included in the analysis due to technical issues. According to this assay, 162 miRNAs were detectable with a Ct value less than 35 in 75% of erythrocyte/reticulocyte samples. Hsa-miR-152 was chosen as the endogenous control for normalization across different samples. A −ΔCt (=Ct_microRNA−Ct_miR-152) was calculated as an equivalent of normalized relative gene expression level for microRNAs and used for hierarchical clustering and other analysis. The choice of hsa-miR-152 as endogenous control is based on two reasons: 1) the level of miR-152 does not alter during a study of microRNAs in erythroid differentiation; 2) hsa-miR-152 showed the least standard deviation (Stdev=0.68) across 40 tissues in a global microRNA profiling study using 210 microRNA TaqMan assays (Applied Biosystems, unpublished data).

In Vitro Maturation Assays of Reticulocytes and miR-320 Knockdown

In order to compare terminal differentiation kinetics between normal and SCD reticulocytes, an in vitro maturation assay was set up to assess their ability to undergo terminal differentiation as described previously (Skadberg et al. (2003) Lab. Hematol. 9: 198-206). Leukopack-filtered erythrocytes were incubated with CD71 microbeads and further processed with an autoMACS™ Separator for the isolation of CD71 positive-young reticulocytes. Flow cytometry and new Methylene Blue stain was performed to validate the purity of the isolated cells. The purified reticulocytes were then cultured in 10% fetal bovine serum-containing RPMI supplemented with 50% of autologous plasma at 37° C. in a humidified atmosphere containing 5% CO2.

Locked Nucleic Acid (LNA) oligonucleotides against miR-320, mir-20a and a scrambled control sequence were obtained from Exiqon (Vedbaek Denmark). LNA oligonucleotides were individually diluted by Opti-MEM, and mixed with diluted Lipofectamine 2000 (Invitrogen) for 30 min at room temperature before transfecting into reticulocytes at a final concentration of 33 nM. Transfection medium was replaced 6 hours post-transfection with the differentiation media composed of RPMI containing 10% FCS and 50% autologous plasma. CD71 expression levels of transfected cells were analyzed by FACS at the indicated time after transfection.

### Cloning of CD71 Reporter Constructs and Luciferase Assays

The 3′UTR of CD71 was amplified from a K562 cDNA with a pair of primers (forward: atgtgatacccatagcttcc (SEQ ID NO: 19); reverse: ggcttagatctcatttggag (SEQ ID NO: 20)) and cloned into the XbaI site downstream of the firefly luciferase gene in the pGL3-control plasmid (Promega, Wisconsin). The CD71 3′UTR miR-320 mutant reporters were constructed with QuikChange® II Site-Directed Mutagenesis Kits (Stratagene, Calif.), which created three base pair changes in the miR-320 seed sequence-targeted regions (underlined) (CTAGATGTCTTTAGGCAGGATCCTTTAA; SEQ ID NO: 21 to replace CTAGATGTCTTTAGGCAGCAGCTTTTAA; SEQ ID NO: 22). To assess the effect of miR-320 on CD71 3′UTR activity, expression constructs encoding miR-320 and miR-451 were inserted into a CMV-based pcDNA3 cloning vector (Invitrogen, CA). The following primers were used to amplify the expression constructs from the genomic DNA of K562 cells, which were then cloned into the XhoI and EcoRI site of pcDNA3: miR-320 (forward: ccgaattccaggaaccagacagggacgc (SEQ ID NO: 23); reverse: ccctcgagccgactcttaagtccaggtc (SEQ ID NO: 24)) and miR-451 (forward: ccgaattcacagtgcttttcaagccatgc (SEQ ID NO: 25); reverse: ccctcgagatcctcctgccttggcctctg (SEQ ID NO: 26)). The functions of these expression constructs have been confirmed by their ability to specifically repress pGL3 luciferase “sensor” constructs comprising two copies of the perfect matched sequences. K562 cells were cotransfected with 1 μg of indicated reporter (CD71 3′UTR or mutCD71 3′UTR), 50 ng renilla luciferase construct, and 2 μg of the indicated microRNA expression constructs, all combined with Lipofectamine 2000 (Invitrogen). After 48 hours, the transfected cells were washed and lysed with the passive lysis buffer (Promega). The luciferase activities (both firefly and renilla luciferase) present in the cell lysates were then determined by Luminometer (Berthold Technology, Germany). The relative reporter activities were obtained by normalization of firefly to the renilla luciferase activities determined in the same lysates with the Dual-Glo Luciferase Assay (Promega)

### Example 1

### Functional microRNAs in Human Mature Erythrocytes

### Discovery of Abundant and Diverse MiRNAs in Human Mature Erythrocytes

To test the possibility that mature erythrocytes contain previously undetected RNA species, we first developed a protocol to obtain a pure population of mature erythrocytes without other contaminating cell types. Blood from healthy volunteers went through a PurecellNeo filter to remove most leukocytes and then a Ficoll-hypaque density gradient to separate erythrocytes from leukocytes and platelets. Any remaining leukocytes (CD45+) and reticulocytes (CD71+) in the packed erythrocyte pellets were further removed via magnetic immuno-depletion in an AutoMac machine to yield the mature erythrocytes (FIG. 1A). The cell purity was confirmed by the surface expression of lineage-specific markers and found to be 99.8% positive for the erythrocyte-marker CD235a while negative for leukocyte (CD45, CD16) and reticulocyte (CD71) markers (data not shown). These FACS results correlated well with the absence of the leukocyte marker CD45 found by RT-PCR (data not shown). The successful depletion of reticulocytes was further confirmed with methylene blue staining (data not shown) and by the Duke clinical reticulocyte laboratory, which found the presence of 0.2-0.7% contaminating reticulocytes in the purified HbAA erythrocytes and 0.5-1.3% contaminating reticulocytes in the purified HbSS erythrocytes stained with acridine orange.

To ensure that we recovered erythrocyte RNAs of all sizes, we chose a miRNA purification method capable of capturing RNAs as small as 10 nucleotides (nt). By this means of RNA purification, we obtained a low but reproducible amount of RNA from the mature erythrocytes of all 53 tested erythrocyte samples. The RNA yields ranged from 5-10 μg RNA from 10 ml of whole blood; by back-calculation, considering the cell numbers and cell purification yield, with the assumption of equal RNAs in all erythrocytes, the RNA content was approximately 2-3×10⁻⁴ pg/cell. This RNA content is similar to the level of RNA found in platelets (3×10⁻⁴ pg/cell), but significantly lower than that found in nucleated cells (5-9 pg/cell) (Bahou et al. (2004) Semin. Thromb. Hemost. 30: 473-484). To rule out the possibility that the RNAs were from contaminating reticulocytes, we adopted an alternative approach to separate reticulocytes from erythrocytes with an arabinogalactan density gradient (Zen et al. (2004) Am. J. Hematol. 75: 63-72). With this independent approach of isolating mature erythrocytes, we obtained a similar level of erythrocyte RNA.

We first determined the size distribution of the RNA with an Agilent bioanalyzer. Erythrocyte RNAs (FIG. 1B, lanes 2-4) did not contain the two distinct ribosomal bands (28S and 18S) observed in all nucleated cells (FIG. 1B, lane 5). Instead, erythrocyte RNAs were significantly enriched for small-sized RNAs less than 200 base pairs (FIG. 1B, lanes 2-4), which are also present in the leukocyte RNA at lower relative abundance. A significant fraction of the erythrocyte RNAs were less than 40 nt. Since most small-sized RNA is lost during many RNA isolation procedures, the unique size distribution of erythrocyte RNA may contribute to the previous perception that they do not contain RNA.

The enriched small-sized RNA species prompted us to test for the expression of miRNAs, which are a family of non-coding RNAs of 19-25 nt that play important regulatory roles in different biological processes (Bartel, D. P. (2004) Cell 116: 281). We utilized a miRNA microarray platform containing DNA oligonucleotides representing more than 196 human miRNAs (Shingara et al. (2005) RNA 11: 1461) to interrogate the microRNA contents of mature erythrocytes and K562, an erythroleukemia cell line which expresses many genes typical of an earlier stage of erythroid differentiation. Equal amounts (40 ng) of fractionated RNAs (<40 nt) from three erythrocyte and two K562 samples were labeled for microarray analysis and revealed abundant and diverse miRNAs. As shown in FIG. 1C, the hybridization result revealed abundant and diverse microRNAs within the erythrocyte RNAs and 87 microRNAs whose expression varied between erythrocyte and K562 more than 3 fold in at least two samples.

When compared with K562, mature erythrocytes specifically expressed several microRNAs, including several microRNAs in the let-7 family and several microRNAs (miR-181a, miR-223, miR-15, miR-16) that have been shown to play a role in the lineage differentiation in the hematopoietic system (Chen et al. (2004) Science 303: 83) (FIG. 1C). In addition, we compared our array results with a recent study of miRNA expression in the hematopoietic CD34+ progenitors (Lu et al. (2005) Nature 435: 834). Despite the fact that CD34+ cells in this study did not reach the terminal stage; many of our erythrocyte-specific miRNAs also showed selective accumulation during early stages of erythroid differentiation (Lu et al. (2005) Nature 435: 834) (FIG. 1C). Similar sets of microRNAs were also found in two other studies of microRNA expression with different in vitro erythroid differentiation models (Choong et al. (2007) Exp Hematol 35: 551-564; and Zhan et al. (2007) Exp Hematol 35: 1015-1025). The expression of these miRNAs was further confirmed with Northern blots and found to be mostly in mature form (FIG. 1D). No significant miRNA degradation products were noted in the Northern blot analysis, making these miRNA unlikely to be partially degraded remnants left over from reticulocytes.

The mRNA of whole blood comes mainly from leukocytes since they contain much more mRNA than erythrocytes and platelets (Whitney et al. (2003) Proc. Natl. Acad. Sci. USA 100: 1896-1901). To test the relative contribution to whole blood miRNA expression of erythrocytes and white blood cells, we purified the miRNA from whole blood, white blood cells and erythrocytes. We then compared the miRNA expression and found that the erythrocyte miRNA expression pattern was much closer to whole blood and arranged in the same cluster by hierarchical clustering (FIG. 2A). We also used a continuous density gradient of 1.093-1.108 g/ml to further separate purified erythrocytes into 8 fractions, roughly corresponding with erythrocyte ages (Lutz et al. (1992) Biochim. Biophys. Acta 1116: 1-10). Although contaminating reticulocytes were present only in the lightest fraction, we still detected significant amounts of miRNA in all other fractions composed of erythrocytes of different ages (data not shown), further confirming the existence of miRNA in mature erythrocytes. This result indicated that the miRNAs of erythrocyte miRNA contributed to the majority of miRNA expression in whole blood. Most miRNAs (miR-20, miR-16, miR-15, let-7, miR-93, miR-107) with specific expression in erythrocytes (FIG. 1C) and whole blood were also highly expressed in erythrocytes when compared with K562 cells (FIG. 2A). This result also raised the possibility of using whole blood miRNA expression as a surrogate for erythrocyte miRNA expression.

We speculated that the expression of erythrocyte miRNAs underwent dynamic alterations during various physiological or pathological processes. We first tested the changes in miRNA expression during erythrocyte terminal differentiation by comparing CD71+ reticulocytes and CD71− erythrocytes. Given the limited number of reticulocytes in normal blood and the low level of RNA in normal reticulocytes, we used a multiplexing stem-loop real-time PCR assay (Lao et al. (2006) Biochem. Biophys. Res. Commun. 343: 85) that exhibits a large dynamic range in minute amounts of samples (Tang et al. (2006) Nucleic Acids Res 34: e9) to assay the miRNA expression levels. Importantly, this technology relies on the specific finding of both the 5′ and 3′ ends of microRNA and therefore detects only full length mature microRNAs but not their precursors or partially degraded products. We used this real-time RT-PCR assay to determine the expression level of 192 mature miRNA in ten erythrocyte/two reticulocyte samples to compare the microRNA expression for erythroid cells at these two different differentiation stages. The reticulocytes were further sorted by FACS from the CD71+ fraction obtained during the process of erythrocyte immunodepletion. Out of 192 assayed miRNAs in real-time RT-PCR, we detected 162 miRNAs (with cycle threshold (CT)<35) in more than 75% of the samples and included these miRNAs for further analysis. This result indicated that the reticulocytes and erythrocytes possess abundant and diverse groups of microRNAs, which were full length mature microRNAs instead of partially degraded microRNA remnants. Out of these 162 microRNAs, which were expressed in erythrocytes, 136 of them were represented on our microRNA microarrays and 130 exhibited hybridization signals in the Cy5 channel more than 2 fold above background. MicroRNAs in the let-7 family were among the most abundant transcripts in real-time PCR assays, consistent with our results from microarrays. We normalized the expression levels of the 162 microRNAs against miR-152 expression levels in all 12 samples for further analysis. We used miR-152 as an endogenous normalization control, since its expression was not altered during different stages of erythroid differentiation (Lu et al. (2005) Nature 435: 834-838), and it showed the least amount of variation across 40 different human tissues in a separate microRNA real-time RT-PCR profiling study (Applied Biosystems, unpublished data). The resulting negative normalized Ct (Cycle of Threshold) values (as represented by −ΔCtᵢ=−(Ctᵢ−Ct_{miR152})) of all remaining 161 miRNA were used to indicate the expression level of each microRNA. The miRNA expression profiles were very different between reticulocytes and erythrocytes and easily separated by unsupervised analysis (FIG. 2B). Therefore, mature erythrocytes have a microRNA expression pattern distinct from that of reticulocytes. We have further applied Significant Analysis of Microarrays (SAM) (Tusher et al. (2001) Proc Natl Acad Sci USA 98: 5116-5121) in a supervised analysis and found that of the 161 expressed miRNAs, 83 were expressed at higher levels in reticulocytes than erythrocytes and termed “recticulocyte-specific miRNAs” (FIG. 2B and Table 1). These reticulocyte-specific miRNAs included microRNAs of the let-7 family as well as miR-221 and miR-222, two microRNAs whose downregulation during erythroid differentiation is essential to this process (Felli et al. (2005) Proc Natl Acad Sci USA 102: 18081-18086).

TABLE 1
The 83 microRNAs with preferential expression in reticulocytes.

| | | | |
|---|---|---|---|
| hsa-miR-125b | hsa-miR-321 | hsa-miR-193 | hsa-miR-145 |
| hsa-miR-128b | hsa-miR-221 | hsa-let-7f | hsa-miR-99b |
| hsa-let-7a | hsa-let-7g | hsa-miR-19a | hsa-miR-122a |
| hsa-miR-146 | hsa-miR-129 | hsa-miR-151 | hsa-miR-373 |
| hsa-miR-188 | hsa-miR-197 | hsa-miR-187 | hsa-let-7d |
| hsa-miR-339 | hsa-let-7c | hsa-let-7e | hsa-miR-338 |
| hsa-miR-143 | hsa-let-7b | hsa-miR-196b | hsa-miR-370 |
| hsa-miR-93 | hsa-miR-345 | hsa-miR-103 | hsa-miR-125a |
| hsa-miR-302a | hsa-miR-196-1 | hsa-miR-198 | hsa-miR-216 |
| hsa-miR-199a | hsa-miR-139 | hsa-miR-196 | hsa-miR-373 |
| hsa-miR-330 | hsa-miR-337 | hsa-miR-10b | hsa-miR-324-3p |
| hsa-miR-222 | hsa-miR-184 | hsa-let-7i | hsa-miR-189 |
| hsa-miR-26b | hsa-miR-196a | hsa-miR-19b | hsa-miR-342 |
| hsa-miR-135a | hsa-miR-199a | hsa-miR-200a | hsa-miR-105 |
| hsa-miR-210 | hsa-miR-107 | hsa-miR-147 | hsa-miR-20 |
| hsa-miR-325 | hsa-miR-98 | hsa-miR-138 | hsa-miR-194 |
| hsa-miR-218 | hsa-miR-23b | hsa-miR-135b | hsa-miR-137 |
| hsa-miR-108 | hsa-miR-15b | hsa-miR-211 | hsa-miR-340 |
| hsa-miR-220 | hsa-miR-124b | hsa-miR-195 | hsa-miR-185 |
| hsa-miR-29a | hsa-miR-302b | hsa-miR-95 | hsa-miR-223 |
| hsa-miR-101 | hsa-miR-30c | hsa-miR-27a | |

miRNA Expression Pattern is Different Between Normal And SCD Erythrocytes

Sickle cell disease (SCD) is caused by a point mutation in the β-chain of hemoglobin, but not all phenotypic abnormalities in SCD erythrocytes can be readily explained by this mutation. We wished to investigate whether expression of erythrocyte miRNA might be altered in SCD and contribute to abnormal phenotypes in SCD. We recruited 12 individuals with HbSS (homozygous sickle cell disease) and 7 race-matched individuals with HbAA (normal hemoglobin). We used the three-step purification method based on both density separation and immuno-depletion of the reticulocyte population to isolate erythrocytes for RNA analysis since the density-only approach may suffer potential reticulocyte contamination in “dense” cell fractions from the HbSS patients. Using FACS, methylene blue stain, and clinical laboratory techniques, we confirmed that the purification scheme would allow us to obtain a mature erythrocyte population from HbSS individuals, who typically have higher levels of reticulocytes than normal individuals. The microRNA expression pattern of the purified erythrocytes from these 19 individuals was determined by microRNA microarrays. We found that the miRNA expression pattern in SCD erythrocytes was distinct from normal erythrocytes based on unsupervised hierarchical clustering (FIG. 2C). Several miRNAs (miR-320, let-7s, miR-181a, miR-141) were over-represented in the normal erythrocytes while other miRNAs (miR-29a, miR-144, miR-451, miR-140) were over-represented in the SCD erythrocytes (FIG. 2C). To compare the differentiation status of these two groups of erythrocytes, we extracted expression values of the reticulocyte-specific miRNAs from all samples (FIG. 2D). We found that the expression of reticulocyte-specific miRNAs was much higher in SCD (FIG. 2C, p<0.0001) than in normal erythrocytes. This result suggested that SCD erythrocytes were less differentiated and exhibited a molecular phenotype closer to reticulocytes, consistent with their relative younger age given that SCD erythrocytes have a shorter life span than normal erythrocytes (˜20 vs. 120 days) (Hoffman et al. (2004) Hematology: Basic Principles And Practice: Churchill Livingstone). There were also many other differences in the microRNA expression patterns of HbSS and HbAA erythrocytes that may not be related to differentiation stages or erythrocyte ages.

To rule out the possibility that this difference in miRNA expression is simply caused by varying degrees of contaminating reticulocytes, we removed all probes corresponding to the 83 reticulocyte-specific miRNAs and performed the clustering analysis based on the remaining “reticulocyte-free” miRNA; this led to the same grouping pattern (data not shown).

To determine the influence of contaminating reticulocytes on gene expression, we did a “doping” experiment by adding a defined amount of reticulocytes to purified erythrocytes. We separated the leukocyte-depleted HbAA blood cells into mature erythrocytes (CD71−, 0.5% reticulocyte by Duke clinical lab) and reticulocytes (CD71+), and added the CD71+ cells into the CD71− population to achieve 1%, 3% and 5% reticulocyte contamination of the erythrocytes. We observed an incremental increase in the RNA yield with increasing numbers of reticulocytes—a 30% increase in the RNA yield with a 5% increase in the reticulocytes, thus leading to the estimate that reticulocytes contain approximately five times as much RNA as do erythrocytes. When the miRNA composition of these samples was determined with microarrays, we found that the added reticulocytes did have a detectable but modest influence on miRNA gene expression, separating the pure erythrocyte samples away from the three “doping” samples in an unsupervised analysis based on only 5 miRNAs (mostly let-7 family), which varied more than 2 fold in 25% of the samples (¼) (data not shown). When considering the 0.5% “background” reticulocytes, this change became detectable with the addition of 1% reticulocytes—and therefore 1.5% of reticulocyte contamination. However, the change caused by the doped reticulocytes was much smaller than the HbSS-HbAA differences and could not explain the expression pattern of HbSS erythrocytes. First, when all 4 samples in the doping experiment were included with the original HbSS and HbAA samples, they were grouped together with all the HbAA samples instead of the HbSS samples (data not shown). When the same filtering criteria (2 fold change also in 25% of the samples ( 6/24)) were used for both the reticulocyte doping and HbSS gene expression, we found a much more dramatic difference (193 miRNA vs. 5 miRNA) in the HbSS samples (data not shown). Therefore, we conclude that the differences in miRNA expression in HbSS erythrocytes are likely to reflect intrinsic differences in erythrocyte miRNA expression instead of varying degrees of contaminating reticulocytes. This modest effect of reticulocytes may be due to the relatively smaller portion of reticulocyte RNA that is miRNA given the abundant mRNA and rRNA, in contrast to the apparent enrichment of miRNA in the erythrocyte populations in the absence of mRNA and rRNA.

Erythrocyte miRNA Expression Analysis Identifies Subtypes of SCD with Distinct Clinical Phenotypes

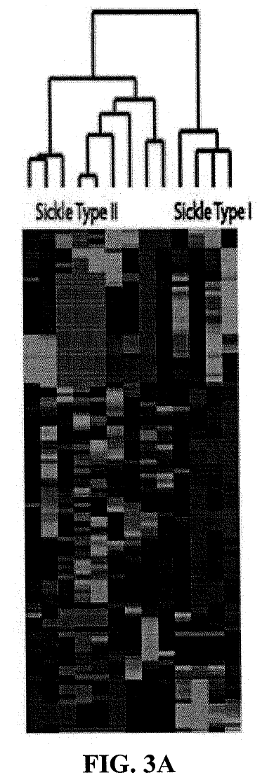

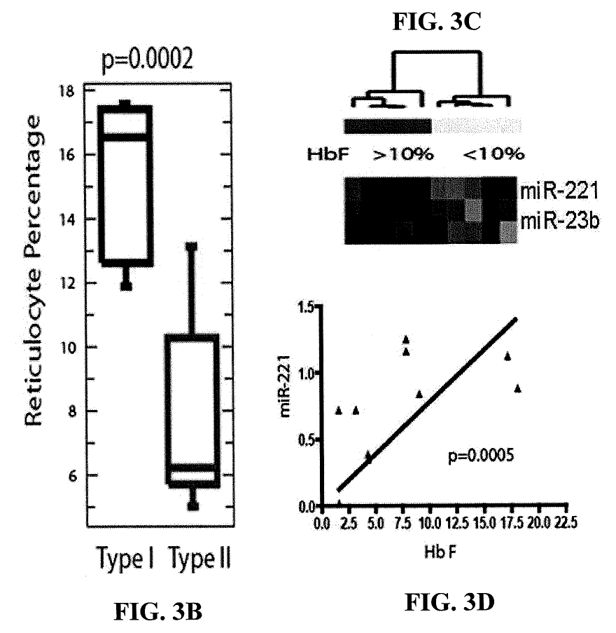

To test whether gene expression patterns will identify subgroups within the HbSS patients with clinical significance, we performed unsupervised analysis of the miRNA expression for class discovery. Two distinct groups were identified based on the 132 miRNAs showing greater than 2-fold variation in at least two samples (FIG. 3A). Four SCD samples were consistently clustered in one branch (termed SCD subtype I) while the rest of the samples were arranged in a separate branch (termed SCD subtype II). This sample separation is very stable with various filtering and selection criteria. When patients in the two groups were compared in terms of their clinical profiles, SCD type I have a much higher reticulocyte percentage than patients with type II expression (15.75% vs. 7.74%, p<0.001) and reticulocyte count (FIG. 3B). Type I patients also have a significantly lower hematocrit and Hb level, suggesting a more severe hemolytic anemia and higher erythropoeisis, possibly consistent with the proposed hemolytic subtypes of SCD (Kato et al. (2006) Blood 107: 2279-2285; and Gladwin, M. T. (2005) Blood 106: 2925). These two groups do not vary significantly by their gender, age, HbF or hydroxyurea use.

Given the importance of HbF in determining the clinical severity of SCD, we applied supervised SAM analysis (Tusher et al. (2001) Proc. Natl. Acad. Sci. USA 98: 5116-5121) to identify miRNA whose expression varied between the two groups, stratified on the basis of a HbF level above or below 10%. Despite the small sample size, we still determined that high levels of miR-23b and miR-221 are associated with a high level of HbF (FIG. 3C). miR-221 was previously shown to inhibit erythrocyte differentiation by downregulating kit receptor (Felli et al. (2005) Proc. Natl. Acad. Sci. USA 102: 18081-18086) and its expression showed a significant correlation with HbF level (FIG. 3D). It is possible that the high miR-221 expression indicates a less differentiated status of the erythrocytes, an observation consistent with their high HbF.

Dysregulated miRNA Expression Leads to Abnormal SCD Phenotypes—Defective Terminal Differentiation

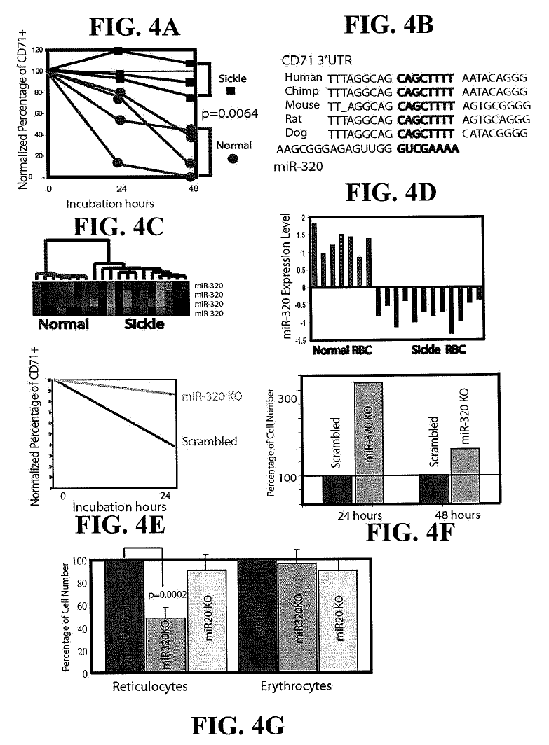

Although reticulocytosis in SCD is thought to be a response to increased erythrocyte turnover, we questioned whether there might also be a defect in reticulocyte maturation in SCD for which microRNAs play a role. CD71 (transferrin receptor) is important both in the capturing of transferrin/iron complexes and the most relevant differentiation marker for the terminal differentiation. Given the importance of RNA-binding proteins in the regulation of CD71 expression at the post-transcriptional level where microRNAs typically function (Eisenstein (2000) Annu Rev Nutr 20: 627-662), we hypothesized that erythrocyte miRNAs might regulate CD71 at the post-transcriptional level. To test this possibility, we set up a terminal differentiation model by placing purified CD71+ reticulocytes in culture with autologous plasma. As previously described, reticulocytes from all four healthy donors underwent terminal differentiation within 48 hours—as evidenced by loss of CD71, disappearance of new methylene blue staining, decrease in mean corpuscular volume (MCV), and an increase in mean corpuscular hemoglobin concentration (MCHC) (FIG. 4A) (Skadberg et al. (2003) Lab. Hematol. 9: 198; and Birney et al. (2004) Nucleic Acids Res. 32 Database issue, D468). In contrast, reticulocytes from all three tested SCD individuals failed to undergo terminal differentiation under the same conditions—they had persistent expression of CD71 (p=0.0064), with no changes in MCV and MCHC during the 48 hours of incubation (FIG. 4A). This result was consistent with our observation that SCD erythrocytes are less differentiated in their miRNA pattern and suggested a potential role for the dysregulated miRNAs in SCD.

miRNAs typically regulate biological processes by forming non-canonical base-pairing with the 3′UTR of target mRNAs (Bartel, D. P. (2004) Cell 116: 281). To identify the miRNAs likely to play a role in CD71 downregulation during maturation, a predictive algorithm was used to search for CD71-targeting miRNAs (Lewis et al. (2005) Cell 120: 15; Krek et al. (2005) Nat Genet. 37: 495-500; and John et al. (2004) PloS Biol 2: e363). MiR-320 was predicted to regulate CD71 by all three predictive algorithms (TargetScans (Lewis et al. (2005) Cell 120: 15), PicTar (Krek et al. (2005) Nat Genet. 37: 495-500), and miRanda (John et al. (2004) PloS Biol 2: e363)) with a perfect match between its “seed” sequence (5′-GUCGAAAA-3′, nucleotides 2-8; SEQ ID NO: 27) and the evolutionarily conserved region in the CD71 3′ UTR (CAGCTTTT; SEQ ID NO: 28; 2693 to 2700 of CD71 mRNA) and additional flanking nucleotides (FIG. 4B). Furthermore, the miR-320 expression level, which was very high in HbAA erythrocytes, was dramatically reduced in SCD (HbSS) erythrocytes (FIG. 4C, D), consistent with a possible role in the defective repression of CD71 seen in HbSS cells. The miR-320 expression level did not change significantly between reticulocyte and erythrocyte samples in our real-time RT-PCR analysis of HbAA cells.

To directly test the functional role of miR-320 during terminal differentiation, we developed a transfection technique that allowed us to elevate and reduce the levels of selected microRNAs in reticulocytes. Using a modified protocol with fluorescently labeled oligonucleotides and lipofectamine, we achieved a transfection efficiency of 40-50% in both erythrocytes and reticulocytes (data not shown. When synthesized mature miR-320 was transfected into reticulocytes, we detected a three-fold increase in miR-320 expression with real-time RT-PCR (data not shown). On the other hand, the transfection of Locked Nucleic Acid (LNA) antisense oligonucleotides against miR-320 led to an 80% reduction in miR-320 levels (data not shown). To test the possibility that miR-320 was essential for CD71 downregulation during terminal differentiation and that low miR-320 in SCD cells might account at least in part for their maturation defect, we inhibited miR-320 function in normal reticulocytes via transfection of LNA against miR-320 before placement in culture for terminal differentiation. The maturation process was intact in the control transfectants with LNA carrying scramble sequences, with the expected down-modulation of CD71. In contrast, miR-320 inhibition led to the persistent expression of CD71 (FIG. 4E) and a higher level of CD71 expression at different time points (FIG. 4F), indicating that miR-320 is indeed essential for CD71 downregulation during terminal differentiation. In addition, miR-320 inhibition caused significant cell death and led to fewer viable cells compared with control LNA or miR-20a inhibition (FIG. 4G). The effect was stage-specific, because mature erythrocytes were not sensitive to the same treatment (FIG. 4G). These results thus revealed the essential role of miR-320 in maintaining erythrocyte homeostasis during terminal differentiation in normal erythroid cells. Phenotypes of miR-320-knockdowns also resembled SCD reticulocytes in their defective maturation and decreased survival. In view of the lower miR-320 expression in sickle erythrocytes, we therefore also concluded that altered miR-320 expression may result in dysregulated maturation and decreased cell survival of erythrocytes in SCD (Hoffman et al. (2004) Hematology: Basic Principles And Practice: Churchill Livingstone).

To evaluate whether CD71 was a direct target of miR-320, we generated CMV-based expression constructs containing the miR-451 and miR-320 genomic sequences and then tested these for their ability to specifically suppress expression from their respective “indicator” reporter constructs containing two copies of identical miR-451 or miR-320 target sequences in a specific manner. A 453 bp region of the CD71 3′UTR (2547-2999 bp of CD71 mRNA) was then amplified and placed downstream of the luciferase reporter gene. When this reporter construct was co-transfected into K562 cells with expression constructs encoding miR-320, miR-451 or empty vectors, we found that only miR-320, but not miR-451 or empty vectors, repressed its activity (data not shown). This miR-320-mediated repression was dependent on the predicted miR-320 target site in the CD71 3′UTR since a three base pair change in this site abolished miR-320-mediated repression (data not shown). Collectively, these results indicated that CD71 was a direct target of miR-320 via the interaction between its 3′UTR and the miR-320 seed sequence.

To further validate the miR-320/CD71 interaction, we overexpressed miR-320 in K562 cells and examined its influence on surface CD71 expression. Overexpression of miR-320 in K562 cells led to a significantly lower level of CD71 when compared with control transfection with empty vectors (data not shown). Taken together, these results showed that miR-320 can directly regulate the expression of CD71 and suggested that the poor expression of miR-320 in HbSS cells is associated with their persistently high CD71 level during terminal differentiation. In addition to CD71, miR-320 was also predicted by TargetScanS (Lewis et al. (2005) Cell 120: 15-20) and PicTar (Krek et al. (2005) Nat Genet. 37: 495-500) to target other mRNAs, including ETS2 (v-ets erythroblastosis virus E26 oncogene homolog 2) and EPB41L5 (erythrocyte membrane protein band 4.1 like 5), two mRNA encoding proteins important for erythrocyte biology. These experiments show that the miRNA composition in erythrocytes can provide a window into the molecular basis of erythrocyte diseased phenotypes seen in SCD.

In this study, we have presented several lines of evidence for the presence of diverse microRNAs in the mature erythrocytes. In gene expression studies of enucleated cells with low RNA content (such as erythrocytes, reticulocytes and platelets) (Goh et al. (2004) Nucleic Acids Res 39: D572-574; Bahou and Ginatenko (2004) Semin Thromb Hemost 30: 473-484; Miller (2004) Blood Cells Mol Dis 32: 341-343; and Gnatenko et al. (2003) Blood 101: 2285-2293), one important concern is that a minority of contaminating nucleated cells with high RNA content (leukocytes) may distort the observed gene expression patterns. We do not believe that such contamination has significantly affected this study, because of the following observations. First, we have employed two different purification schemes, composed of three levels of cell separation based on cell density and surface expression to remove most leukocytes, platelets and reticulocytes, to obtain the mature erythrocytes used for expression analysis. We confirmed the purity of the resulting mature erythrocytes by FACS, methylene blue staining and molecular analysis of isolated RNAs. Second, the size distribution and characteristics of the isolated erythrocyte RNAs were significantly different from that of all nucleated cells—uniquely enriched for small RNA species without prominent bands of ribosomal RNAs. Third, when microRNAs from whole blood were compared with leukocytes and mature erythrocytes, the expression pattern of whole blood, even without leukocyte depletion, was derived mainly from erythrocytes instead of leukocytes. The microRNA expression pattern of mature erythrocytes was also different from reticulocytes using microRNA real-time RT-PCR assays. Taken together, these results indicate that the expression of these microRNAs is indeed from mature erythrocytes and not from other “contaminating” cells. Knowledge of erythrocyte microRNA is also important to investigators who are interested in leukocyte microRNAs, as it is now clear that they must avoid the significant and unexpected bias caused by erythrocyte microRNAs.

The human erythrocyte is one of the most studied cell types and is often used as a model system to understand the general principles of molecular genetics, biochemistry, membrane biology and cell physiology. However, our understanding of erythrocytes and the diseases that affect them is still incomplete. The unexpected discovery of microRNAs in erythrocytes, consistent with a previous study of parasitized red cells (Rathjen et al. (2006) FEBS Lett 580: 5185-5188), adds a new dimension to erythrocyte characterization and has the potential to enhance our understanding of their phenotypic alterations during physiological and pathological adaptations. As a step toward realizing this potential, we used microarrays to analyze erythrocyte microRNA gene expression in SCD to establish a connection between microRNA dysregulation and certain disease phenotypes of SCD. Several recent studies have analyzed microRNA expression on a global scale during in vitro erythroid differentiation (Choong et al. (2007) Exp Hematol 35: 551-564; Zhan et al. (2007) Exp Hematol 35: 1015-1025; and Georgantas et al. (2007) Proc Natl Acad Sci USA 104: 2750-2755). Although these differentiation models do not lead to terminally differentiated erythrocytes analyzed in our study, we note a high degree of similarity in the microRNAs seen during the earlier stages of erythroid differentiation investigated in these studies and the microRNAs reported here in mature erythrocytes. This similarity indicates that the microRNAs present in mature erythrocytes are likely to originate from cells in earlier differentiation stages prior to nuclear exclusion and persist in the mature erythrocyte after terminal differentiation. The selective presence of these microRNAs in these post-mitotic erythrocytes may be due to the longer half-life and slower decay kinetics of specific microRNAs. Our results indicated that microRNAs still exist in the functional form in reticulocytes, consistent with a previous study (Wang et al. (2006) Mol Cell 22: 553-560). The immediate precursors of reticulocytes, orthochromatic normoblasts, still contain nuclei. Since the reticulocyte stage lasts for 40 hours (Hoffman et al. (2004) Hematology: Basic Principles and Practice: Churchill Livingstone), it is conceivable that the reticulocyte microRNAs synthesized in the orthochromatic normoblast become processed, fed into the maturation pathways and persist in a functional form in reticulocytes. These microRNAs may persist in the mature erythrocytes after terminal differentiation.

The main functional mechanism of microRNAs is through the post-transcriptional regulation of their target mRNA via mRNA degradation or translation inhibition (Bartel D P (2004) Cell 116: 281-297). What are the potential roles for microRNAs in mature erythrocytes, a cell type with limited target mRNA and translation activity? What is the clinical relevance for genomic analysis of erythrocyte microRNAs? First, these erythrocyte microRNAs are likely to play important regulatory roles during the earlier stages of erythropoiesis, as late as the reticulocyte. Translational activities are still present in the reticulocyte stage, and our results also clearly demonstrate a functional role of microRNAs in these cells during terminal differentiation of reticulocytes. This observation is consistent with a recent study recapitulating the microRNA-mediated translation repression in reticulocyte cell lysates (Wang et al. (2006) Mol Cell 22: 553-560). Thus, the microRNA composition captured in circulating erythrocytes is the balance between microRNA accumulation from earlier differentiation stages and the subsequent microRNA decay in the remaining erythrocytes exposed to various physiological and pathological conditions. Many other studies have highlighted the functional importance of several microRNAs (e.g. miR-221, -222, -451 and -24) during various stages of erythroid differentiation (Zhan et al. (2007) Exp Hematol 35: 1015-1025; Felli et al. (2005) Proc Natl Acad Sci USA 102: 18086-18086; and Wang et al. (2008) Blood 111: 588-595). Second, erythrocyte microRNAs may play a direct role in the de-adenylation and degradation of mRNA during erythrocyte terminal differentiation as suggested by several studies (Farh et al. (2005) Science 310: 1817-1821; Giraldez et al. (2006) Science 312: 5770; and Wu et al. (2006) Proc Natl Acad Sci USA 103:4034-4039). Third, it is possible that microRNAs play a role in the host-pathogen interaction between erythrocyte and malaria parasites, given that human microRNAs are found in the malaria parasites P. falciparum (Rathjen et al. (2006) FEBS Lett 580: 5185-5188). Similar involvement of microRNAs in the host-pathogen interaction was shown for host microRNAs affecting the growth and propagation of pathogens (Jopling et al. (2005) Science 309: 1577-1581; and Lecellier et al. (2005) Science 308: 557-560) as well as pathogen-encoded microRNAs impacting host cellular physiology (Browne et al. (2005) Genome Biol 6: 238; Pfeffer et al. (2004) Science 304: 734-736; and (Gottwein et al. (2007) Nature 450: 1096-1099). In our analysis, HbSS erythrocytes have a very high level of miR-451, which was found be translocated into P. falciparum. This observation leads to the possibility that microRNA composition may contribute to the malaria resistance noted for sickle cell erythrocytes (Pasvol et al. (1978) Nature 274: 701-703; and Friedman (1978) Proc Natl Acad Sci USA 75: 1994-1997). Finally, MicroRNAs may have additional functional roles in erythrocytes via a mechanism independent of mRNA targeting as suggested in our observation that the blockage of miR-320 leads to decreased erythrocyte survival.

The differential expression of erythrocyte microRNAs may also lead to novel insights into human erythrocyte diseases. We observed a dramatic and significant difference between microRNA expressions in mature HbSS and HbAA erythrocytes; partly because HbSS erythrocytes are younger than HbAA erythrocytes in their microRNA expression pattern, consistent with their shorter life span. These differences are likely to provide additional information about the disease phenotypes, similar to the miR-320::CD71 connection established in our current study. Linkage of particular microRNAs to particular SCD disease phenotypes offers the potential not only to develop useful biomarkers as suggested in a recent review (Zhang and Farwell (2008) J Cell Mol Med 12: 3-21), but also to generate testable biological hypotheses and relevant pathological insights based on these microRNAs and their predicted target mRNAs. This line of research can lead to an enhanced understanding of the relevant pathophysiological mechanisms as well as suggest treatments tailored for individual patients. It is important to expand these studies to a larger cohort of patients with detailed analysis of their biochemical parameters and clinical phenotypes to determine the robustness and stability of the various subtypes and their connection with genetic polymorphisms and other genetic parameters. We expect that similar use of microarrays and advanced bioinformatics will greatly enhance our understanding of pathophysiological mechanisms in various erythroid diseases involving abnormal genetic regulation of erythropoiesis (i.e., aplastic anemia or polycythemia vera), as well as erythrocyte-related dysfunction or hemoglobinopathies (i.e. hemolytic anemia, sickle cell disease).

Without novel mRNA synthesis, post-transcriptional regulation is probably the main mechanism for gene regulation in enucleated cells (reticulocytes, platelets, and erythrocytes). Given the programmed transcriptional arrest with nuclear exclusion in developing erythroid cells, significant gaps sometimes exist between the time of mRNA synthesis and actual translation. RNA stability and translation control are therefore tightly regulated to ensure proper control of RNA stability and protein synthesis (Waggoner and Liebhaber (2003) Experimental Biology and Medicine 228: 387-395), both of which are likely to be regulated by microRNAs. For example, the post-transcriptional control of CD71 is mediated by the actions of iron regulatory proteins (IRP1 and IRP2) in response to iron levels (Eisenstein (2000) Annu Rev Nutr 20: 627-662). The loss of surface expression of CD71 during erythrocyte terminal differentiation was thought to be caused by its release/secretion by exosomes (Johnstone (1989) Blood 74: 1844-1851). Our findings suggest that microRNAs are also involved in the reticulocyte terminal differentiation process. Since miR-320 expression does not change during the transition between reticulocyte and erythrocytes, its upregulation is not likely to be a trigger for the loss of CD71 expression. Instead, miR-320 is likely to fine tune the translational activities of CD71 in reticulocytes and contribute to its loss of CD71 surface expression together with the exosome release. MicroRNA-mediated repression can also be regulated by interactions between miRNA/Argonaute complexes with RNA-binding proteins that relocate from different subcellular compartments during stress or differentiation (Leung and Sharp (2007) Cell 130: 581-585). Many additional mRNAs encode proteins with important regulatory roles in erythropoiesis are also predicted targets of erythrocyte microRNAs (Lewis et al. (2005) Cell 120: 15-20) and their interactions deserve further exploration in the context of various physiological and pathological adaptations of erythrocyte diseases.

### Example 2

### The Role of MiRNAs in the Host-Pathogen Interaction Between Erythrocytes and Plasmodium falciparum

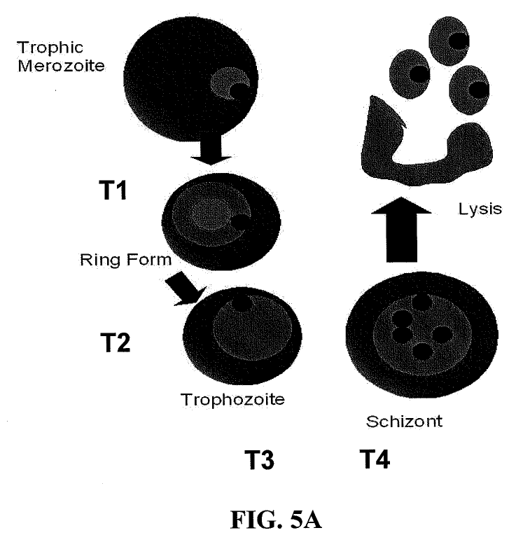

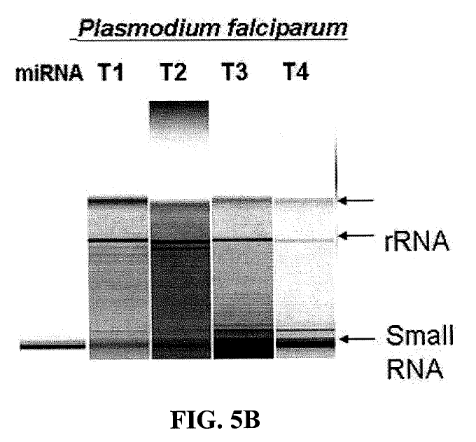

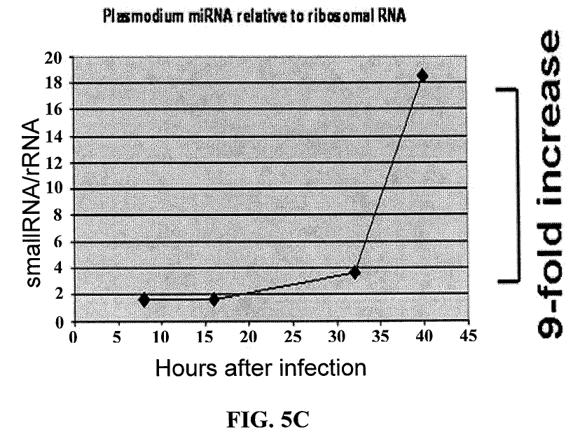

During the intraerythrocytic life cycle of malaria parasite P. falciparum, significant material exchanges occur between the erythrocyte and parasite (Barkan et al. (2000) Int. J. Parasitol. 30: 649-653). To test the possibility that erythrocyte miRNA plays a functional role in erythrocyte-Plasmodium infection, we first determined whether certain erythrocyte miRNAs are found in the parasite. We placed synchronized P. falciparum into washed fresh erythrocytes and maintained them in a hypoxic atmosphere with 5% O₂, 5% CO₂, and 90% N₂. Erythrocyte-free parasites were obtained by selective lysis with 0.15% saponin and the total (including small) RNAs contained in the parasites were collected at 8, 16, 32, and 40 hours, roughly corresponding to early ring, late ring, late trophozoite, and middle schizont stages of intraerythrocytic infection (FIG. 5A). When we analyzed the size distribution of parasite RNAs, we found that small-sized RNAs accumulated (up to 9 fold when normalized against P. falciparum ribosomal RNAs) in the later stages of infection (FIG. 5B, C).

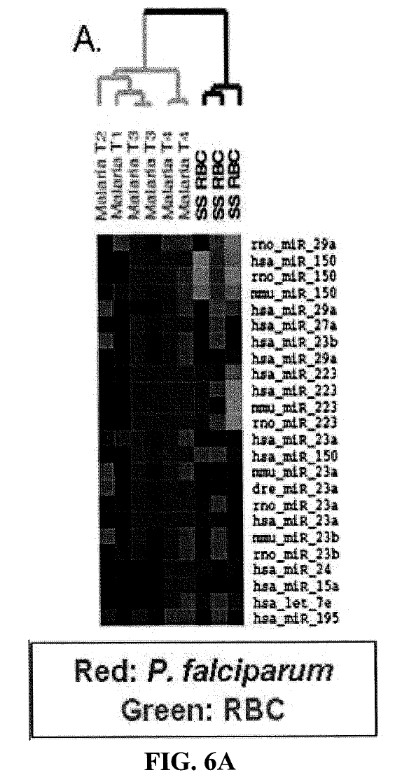

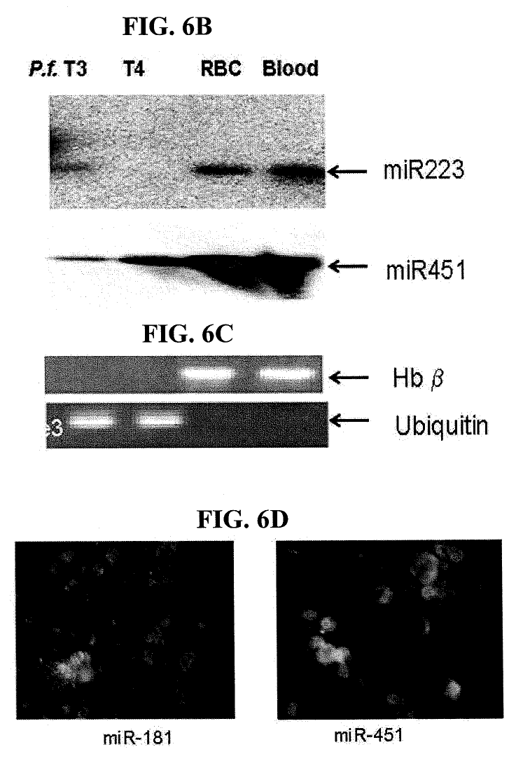

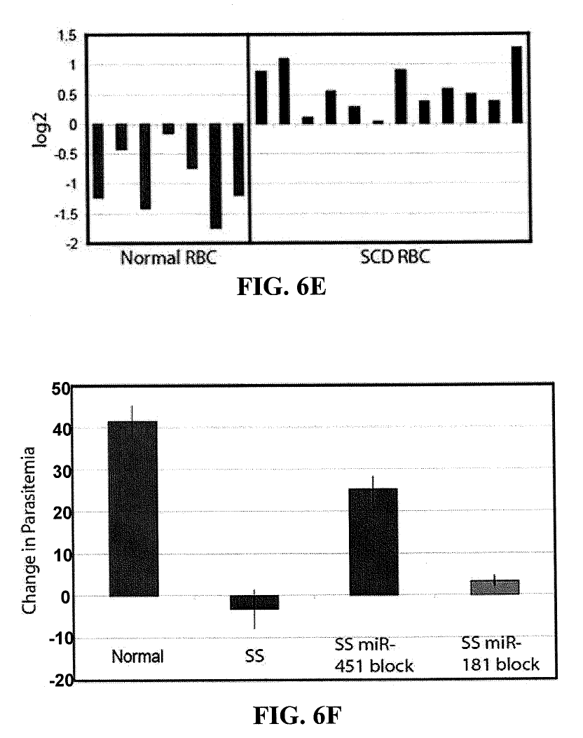

We then used miRNA microarrays for human miRNAs to analyze the fractionated RNAs (<40 nt) from both the infected erythrocytes and parasites to characterize the erythrocyte miRNA found in the parasites. Only a limited set of erythrocyte miRNAs (around 10 based on hybridization signals above 3-fold) were found in the parasites while some very abundant miRNAs (FIG. 6A) and Hb RNAs were not found in the parasites (FIG. 6C). High concentrations of miR-223 and miR-451 were consistently observed in microarray results at late infection stages (T3/T4) (FIG. 6A). Hybridization to miRNA orthologs from multiple species provided further evidence of the consistency of the observation. This translocation is sequence-specific since it only occurs for a small set of miRNAs with no relationship to their abundance. It is also stage-specific since it only occurs at the later stage of infection. This finding is also consistent with one published report showing that the sequencing of miRNA-sized RNAs in P. falciparum led to the identification of miR-451 within parasites (Rathjen et al. (2006) FEBS Lett. 580: 5185-5188). The translocation of miR-451 and miR-223 into the P. falciparum was further confirmed with Northern blots (FIG. 6B), and immunohistochemistry of transfected biotinylated miRNAs (FIG. 6D). To rule out the possibility of erythrocyte contamination, we used a multiplexed TaqMan real-time PCR platform to compare the expression of 332 miRNAs in both uninfected erythrocytes and erythrocyte-free P. falciparum at the indicated time. The relative percentage of both miR-223 and miR-451 in total miRNA in the malaria parasites showed significant enrichment: miR-223 (<1% (in erythrocyte) vs. 13% (in parasite)) and miR-451 (1.6% (in erythrocyte) vs. 12.5% (in parasite)). Although this selective translocation of miRNAs is unexpected, it is consistent with the recent findings of selective nuclear localization of miR-29b in the nuclei (Hwang et al. (2007) Science 315: 97-100).

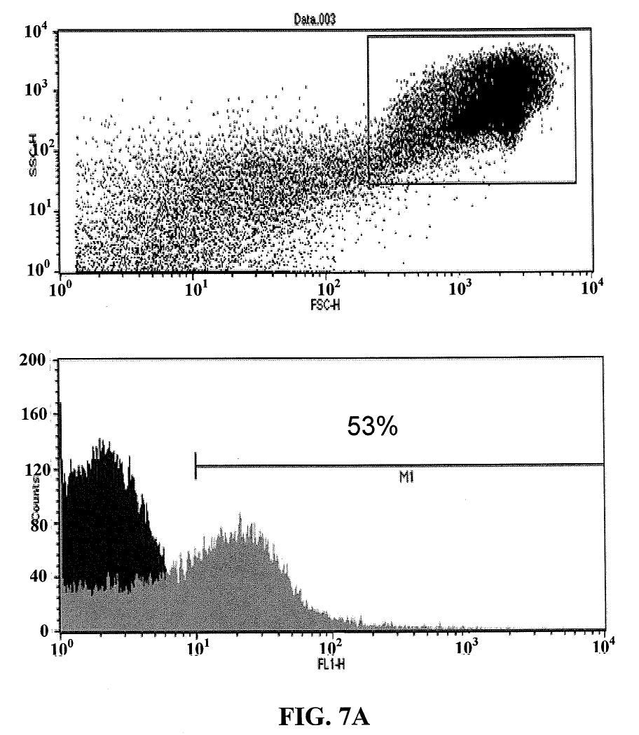

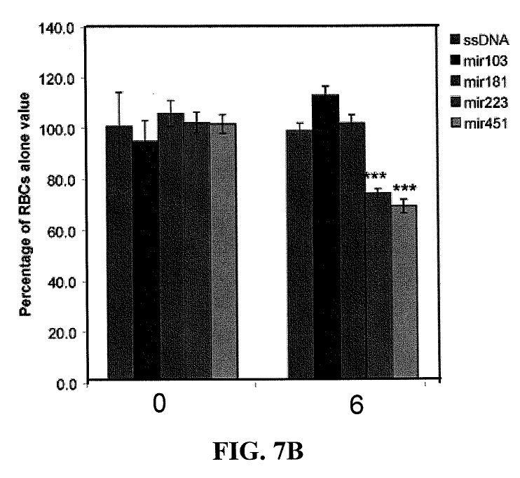

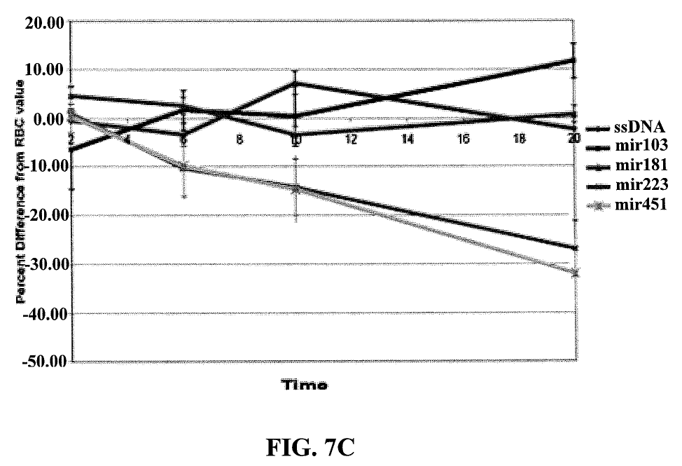

To test for functional effects of these miRNAs on parasite growth, we developed a transfection technique to deliver oligonucleotides into mature erythrocytes with electroporation (van den Hoff et al. (1992) Nucleic Acids Res. 20: 2902). We routinely obtain a transfection efficiency of 50-60% with 200 μl of packed erythrocytes (approximately 1.5×10⁹ cells) (FIG. 7A) that yields enough transfected cells to evaluate the impact on the growth of P. falciparum. The synchronized Plasmodium parasites were grown in the erythrocytes transfected with translocated miRNA (e.g. miR-451, -223) or untranslocated miRNA (e.g. miR-181a, -103) and DNA-oligos. We then evaluated the effects of miRNA overexpression or inhibition by comparing the degrees of parasitemia seen in the transfected erythrocytes. Parasitemia was assessed with fluorescence activated cell sorting (FACS) after the parasites in the permeabilized infected erythrocyte were stained with YoYo-1 (Barkan et al. (2000) Int. J. Parasitol. 30: 649-653). We observed that overexpression of both miR-451 and miR-223 in erythrocytes led to significant decline in parasitemia when compared with control miRNA (miR-181a, -103) or DNA oligonucleotides. This suppression of parasite growth also became more prominent as the parasites were continuously propagated in the transfected erythrocytes (FIG. 7B, C).

It is believed that variations in miRNA expression between normal and sickle erythrocytes lead to their different susceptibility to P. falciparum. From our genomic analysis of erythrocyte miRNAs, we found that miR-451 is over-expressed in all HbSS erythrocytes while miR-223 is expressed in 30% of the HbSS patients (FIG. 6E).

### Example 3

### The Identification of Erythrocyte MiRNAs Distinguishing SCD Subtypes and Other Anemia Disorders

Based on our finding that human mature erythrocytes possess abundant and diverse miRNAs, we aim to bring state-of-the-art genomic tools and advanced bioinformatics to analyze the erythrocytes of SCD and other anemia disorders to gain a deeper understanding of their clinical heterogeneity and erythrocyte phenotypes. We will also identify miRNAs associated with particular phenotypes of SCD.

As noted above, erythrocyte miRNA provides information about the developmental history and turnover kinetics of erythrocytes, we reason that their composition may thus indicate the severity, features and clinical phenotypes of the erythrocyte disorders. In this aim, microarray is used to capture the global miRNA expression and various bioinformatics tools are used to analyze and extract the biological information. HbSS samples are analyzed to determine HbSS subtypes with significant differences in clinical phenotypes based on miRNA expression. The erythrocyte miRNAs of HbSA and other anemia disorders with hemolysis are analyzed to dissect the molecular mechanism underlying miRNA dysregulation in HbSS erythrocytes. Furthermore, we will identify miRNAs which can reliably distinguish between HbSS subtypes as well as other anemia conditions and confirm their expression with TaqMan RT-PCR assays.

The Identification of Erythrocyte miRNA Expression which Predict Phenotypic Subtypes of HbSS Patients

These experiments: 1) identify subtypes among HbSS patients with unsupervised analysis of the global erythrocyte miRNA expression 2) use supervised analysis to identify miRNAs associated with variations in important clinical features of these HbSS patients. We are analyzing 47 additional patients with HbSS from the Duke Comprehensive Sickle Cell Center (CSCC). We are using the miRNA expression of 40 samples as training sets for discovering the HbSS subtypes and building predictive models and identifying specific miRNAs separating these two subtypes.

Patient enrollment—Potential research participants are contacted in person during regular clinic visits, according to available patient rosters, at comprehensive sickle cell clinical programs at Duke University. Informed consent is obtained, and a total of 42 patients are enrolled. Once informed consent is given, patients are interviewed to obtain a medical history and undergo a routine physical examination. A blood sample is collected in order to obtain red blood cells and plasma for further analysis. Laboratory values, including the measurement of reticulocyte count, HbF, and several serum markers indicating hemolysis (e.g. LDH, ASH, and bilirubin) are collected to correlate with their miRNA expression. Finally, available medical records are reviewed by clinical study personnel to obtain specified historical data (e.g. baseline hemoglobin value, the occurrence and the severity of clinical complications).

a) Inclusion and Exclusion Criteria

Patients included in this study are both male and female patients with previously diagnosed HbSS (homozygous) without additional hemoglobin mutations. All patients enrolled are ages 19-50. Patients enrolled are willing to and capable of consenting to participation in the study. Patients are excluded from the study if they received transfusion therapies or experienced clinical crisis during the past three months; are unwilling to comply with the enrollment interview, physical exam, and laboratory studies; do not have on record suitable baseline laboratory data required for inclusion into the study; or if they have other clinical manifestations/complications.

b) Data Collection and Sample Processing Procedures

The mature erythrocytes are purified as described before (FIG. 1A). Total RNA containing small-sized RNA is extracted using the mirVana miRNA Isolation Kit (Ambion), which can capture RNAs as small as 10 nt. For miRNA expression profiling by microarray, the small sized (<40 nt) RNAs are first isolated with FlashPAGE and then labeled using miRCURY™ LNA Array Labeling Kit (Exiqon). The fluorescently labeled sample and common reference miRNAs are then hybridized with printed miRNA microarrays provided by the Duke Microarray Facility. The common reference miRNA used in this study is obtained from one unit of whole blood, allowing the comparison of all the microarray data obtained in this study. The hybridization signals of the arrays are scanned and analyzed using the GenePix software package (Axon). These data are normalized and submitted into the Duke Microarray Database (DMD) found at the website, dmd.duhs.duke.edu/. To access the website, the site should be entered following http://. Data are normalized globally per array, such that the average LogRatio is 0 after normalization. All data used for analysis has an average sum intensity 2.5 fold above either channels, and a regression ratio>0.6 before further filtering based on indicated variations among different samples. Hierarchical clustering is performed by average linkage using uncentered Pearson correlation. This general analytic approach has been extensively used in previous studies (Chang et al. (2002) Proc. Natl. Acad. Sci. USA 99: 12877-12882; Chang et al. (2004) PLoS Biol. 2: E7; Chi et al. (2003) Proc. Natl. Acad. Sci. USA 100: 10623-10628; Chi et al. (2003) Proc. Natl. Acad. Sci. USA 100: 6343-6346; and Chi et al. (2006) PLoS Med. 3: e47) and described in more detailed below.

c) Data Analysis

To analyze the erythrocyte miRNA expression obtained by microarrays, we are first performing unsupervised analysis with hierarchical clustering (Eisen et al. (1998) Proc. Natl. Acad. Sci. USA 95: 14863-14868) for “class discovery”. In addition to its ability to give us a general understanding of the variation and architecture of the miRNA expression in the samples, unsupervised analysis can also identify the heterogeneity of the samples based on the expression data. It has led to the concept of “molecular diagnosis” (Berns, A. (2000) Nature 403: 491-492) and the discovery of important subtypes of B-cell lymphoma (Alizadeh et al. (2000) Nature 403: 503-511), breast cancer (Perou et al. (2000) Nature 406: 747-752), and lung cancers (Garber et al. (2001) Proc. Natl. Acad. Sci. USA 98: 13784-13789) unrecognized with traditional histopathology. When we applied this approach to analyze the erythrocyte miRNA expression from the 12 HbSS patients, we clearly identified two subgroups of the SCD with different amounts of reticulocytes, Hb, Hematocrit, and erythrocyte level (FIG. 3A, B). This observation suggests a different severity of anemia and the possibility of SCD type I corresponding to the hemolytic subtypes of SCD. Here, we are collecting more patients to achieve two goals 1) test whether this classification is robust, stable, and allows us to build a predictive model 2) correlate gene expression-based subtypes with the level of LDH, ASH and other parameters indicating hemolysis (Kato et al. (2006) Blood 107: 2279-2285).

In addition, we are also using supervised analysis for “feature identification”. Supervised analysis allows us to identify miRNAs associated with a particular individual phenotype against all confounding technical or irrelevant biological variables. The identification of miRNA tightly linked to particular phenotypes can lead to the development of diagnostic tools and a better understanding of the underlying pathophysiological mechanisms. Two supervised analyses are used here: SAM (Tusher et al. (2001) Proc. Natl. Acad. Sci. USA 98: 5116-5121) and rank-sum test (Chi et al. (2003) Proc. Natl. Acad. Sci. USA 100: 10623-10628). We are first focusing on the following erythrocyte phenotypes: 1) SCD subtypes found with unsupervised analysis; 2) reticulocyte level; 3) HbF level; 4) hydroxyurea treatment 5) anemia severity (Hb, hematocrit); and 6) serum markers indicating hemolysis (LDH) (Kato et al. (2006) Blood 107: 2279-2285). Furthermore, we are investigating other clinically important complications, such as pulmonary artery hypertension, priapism, and renal damage (evidenced by proteinuria and reduced GFR). Many of these non-erythrocyte complications are also associated with erythrocyte hemolysis and NO scavengering (Kato et al. (2006) Blood 107: 2279-2285; Gladwin (2005) Blood 106: 2925; and Hsu et al. (2007) Blood 109: 3088-3098). Even when these phenotypes are not directly related to erythrocytes, the analysis of erythrocyte miRNA may provide value based on the idea of “blood sensors”—similar to the use of gene expression in circulating blood/PBMCs to inform the disease severity of cardiovascular diseases or cancers (Critchley-Thorne et al. (2007) PLoS Med 4: e176; Osman et al. (2006) Clin. Cancer Res. 12: 3374-3380; Learn et al. (2006) Clin. Cancer Res. 12: 7306-7315; Simon (2005) J. Clin. Oncol. 23: 7332-7341; Stremmel et al. (2005) Int. J. Colorectal Dis. 20: 485-493; Burczynski et al. (2005) Clin. Cancer Res. 11: 1181-1189; Sharma et al. (2005) Breast Cancer Res. 7: R634-644; Xu et al. (2004) Cancer Res. 64: 3661-3667; Lotze and Rees (2004) Cancer Immunol. Immunother. 53: 256-261; Twine et al. (2003) Cancer Res. 63: 6069-6075; Shingyoji et al. (2003) Cancer 97: 1057-1062; DePrimo et al. (2003) BMC Cancer 3: 3; and Stremmel et al. (2002) Int. J. Colorectal Dis. 17: 131-136). Dr. Telen has recently published two studies on genetic polymorphisms associated with pulmonary artery hypertension and priapism (Elliott et al. (2007) Br. J. Haematol. 137: 262-267; and De Castro et al. (2007) Am. J. Hematol.) The erythrocyte miRNA is profiled for many of the same patients for whom we have information about genetic polymorphisms and clinical phenotypes. This supervised approach has led to the identification of miR-221 and miR-23b associated with HbF level (FIG. 3C, D). In fact, the expression levels of both miRNAs show a statistically significant positive correlation with HbF level (FIG. 3D and data not shown). Although the mechanism underlying the link remains to be determined, the role of miR-221 in downregulating kit receptor and inhibiting erythropoeisis might be relevant (Felli et al. (2005) Proc. Natl. Acad. Sci. USA 102: 18081-18086). This is an example of using the supervised approach to identify specific miRNAs associated with the investigated phenotypes of SCD. Similar approaches are used to analyze the miRNAs associated with the listed phenotypes as a starting point to develop hypotheses about the molecular mechanisms underlying the linkages.

To rule out the possibility that our microarray analysis is affected by contaminating reticulocytes, we are also removing all the 83 reticulocyte-specific miRNAs from the microarray results before analysis. By doing this, we can further test whether our biological conclusions made by the analysis of all miRNAs are still present when all reticulocyte miRNAs are removed. The selection of non-reticulocyte specific miRNAs can be easily done in the Duke Microarray Database as has been done in several previously published studies (Chang et al. (2002) Proc. Natl. Acad. Sci. USA 99: 12877-12882; Chang et al. (2004) PLoS Biol. 2: E7; Chi et al. (2003) Proc. Natl. Acad. Sci. USA 100: 10623-10628; Chi et al. (2003) Proc. Natl. Acad. Sci. USA 100: 6343-6346; Chi et al. (2006) PLoS Med 3: e47; and Chi et al. (2007) PLoS Genet. 3: 1770-1784). For example, we have found that the separation between HbSS vs. HbAA and two HbSS subtypes persisted when only non-reticulocyte miRNAs were analyzed.

We anticipate that ˜30% of patients belong to the type I SCD given our data in the pilot samples. Therefore, we are enrolling a total of 60 patients into the study, with 18 type I and 42 type II SCD patients. In the training dataset, we should have 12 type I sample and 28 type II sample, which should give us enough statistical power to identify candidate miRNAs with the ability to distinguish different subtypes.

The Identification of Erythrocyte MiRNAs which Distinguish Patients with HbSS, Sickle Cell Traits (HbSA,) and Other Hemolytic Conditions not Caused by HbS

Two potential sources of abnormal miRNA expression in HbSS are: 1) HbS; and 2) the increased erythropoeisis in response to hemolysis. We are identifying the contribution of these two factors by comparing the HbSS samples with two additional groups of patients—patients with sickle cell trait (HbSA) and patients with increased erythropoeisis due to hemolytic conditions not caused by HbS, such as PNH. This way of decomposing individual factors via integrative genomic analysis has been applied in cancer biology (Chang et al. (2004) PLoS Biol. 2: E7; Chi et al. (2006) PLoS Med. 3: e47; and Bild et al. (2006) Nature 439: 353-357) and used in understanding the differentiation status of the HbSS erythrocytes (FIG. 2D).

We are first collecting the erythrocyte miRNA from patients with sickle cell trait (HbSA). Sickle cell trait (HbSA) usually is not regarded as a disease state because the complications are either uncommon or mild. Nevertheless, patients with HbSA still have the problem of polymerization of deoxy-Hb S since serious morbidity or mortality can result from complications with processes that cause hypoxia, acidosis, dehydration, hyperosmolality, hypothermia, or elevated erythrocyte 2,3-DPG. These conditions can transform a silent sickle cell trait into a syndrome resembling sickle cell disease with vaso-occlusion due to rigid erythrocytes (Embury et al. (1994) Sickle Cell Anemia: Basic Principles and Clinical Practice: Lippincott Williams & Wilkins. 901 p). This association of miRNA expression with HbS is likely due to the HbS polymerization or the physical linkage with particular HbS haplotypes (Embury et al. (1994) Sickle Cell Anemia: Basic Principles and Clinical Practice: Lippincott Williams & Wilkins. 901 p). We are collecting 20 individuals who have been tested to be HbSA by Hb electrophoresis from the families of HbSS patients in the Duke CSCC. We are using 15 HbSA for array analysis and 5 samples for validation purposes. Since these patients are likely to be genetically related to the HbSS patients recruited for our study, it is important to document this relationship so we can identify the miRNAs associated with HbSS vs. HbSA instead of with a particular family.

We also anticipate that a significant portion of SCD miRNA expression is caused by the increased erythropoeisis caused by hemolysis due to several reasons. These miRNAs may be involved in regulating erythropoeisis (either enhancing or inhibiting) (Felli et al. (2005) Proc. Natl. Acad. Sci. USA 102: 18081-18086) and are upregulated during increased erythropoeisis. This will lead to elevated levels of these miRNAs in mature erythrocytes. It is also possible that erythrocyte miRNA may influence the tendency of erythrocytes to undergo hemolysis. In this aim, we are collecting the RNAs from mature erythrocytes from 10 patients of Paroxysmal nocturnal Hemoglobinuria (PNH) and 10 patients of other anemias characterized by hemolysis (such as autoimmune hemolytic anemia). These anemia disorders all share the hemolytic feature with SCD, but the basis for the hemolysis are distinct from SCD. PNH is caused by a mutation in GPI-anchor protein (Takeda (1993) Cell 73: 703-711) and this mutation leads to hemolysis and marrow failure (Parker et al. (2005) Blood 106: 3699-3709). Dr. Carlos DeCastro, the current director of the Duke PNH center (Nishimura et al. (2004) Medicine (Baltimore) 83: 193-207; Heeney et al. (2003) Mol. Genet. Metab. 78: 291-294; and Ware et al. (2001) Exp. Hematol. 29: 1403-1409), is assisting with the recruitment of appropriate PNH patients entering this study. We are obtaining the patients with other anemias from different hematologists in Duke Medical Center, including Drs. Telen and DeCastro. The patient recruitment procedures, sample handling and the array analytic approaches will be similar to the handling of the SCD erythrocyte samples.

When the erythrocyte miRNA expression of 7 PNH patients was included in an analysis of all samples, the original separation of HbAA and HbSS into two distinct branches persisted (data not shown). Interestingly, the 7 PNH samples were split into two branches, one branch (four samples) arranged together with HbAA (termed “normal-like” group) and another branch (three samples) arranged with HbSS (termed “HbSS-like”) individuals (data not shown). Patients with the “normal-like” gene expression had a low level of clinical hemolysis when compared with patients with the “HbSS-like” gene expression. In fact, three patients in the “normal-like” group were receiving eculizumab treatment with a corresponding decline in hemolysis. The fourth patient had very mild clinical hemolysis. In contrast, the three PNH patients in the “HbSS-like” group exhibited significant clinical hemolysis. This result showed that the erythrocyte miRNA gene expression pattern could separate the patients with PNH into groups with distinct clinical phenotypes (data not shown). It also indicated that one important component of dysregulated gene expression seen in HbSS erythrocytes was hemolysis, a clinical feature shared by all HbSS and a subset of PNH patients.

Supervised SAM analysis allowed the identification of 256 erythrocyte miRNAs associated with one of the three groups (either HbAA, HbSS, or PNH) with a false discovery rate (FDR) of 4.3% (data not shown) (Tusher et al. (2001) Proc. Natl. Acad. Sci. USA 98: 5116-5121). The PNH-specific genes included miR-17 and miR-130a. The HbSS-specific genes included miR-223 and miR-451, both of which are found in Plasmodium falciparum parasites. The normal-specific genes included miR-320, whose defective expression in HbSS reticulocytes was linked to the failure of terminal differentiation (FIG. 4). Although miR-320 expression in PNH erythrocytes was also reduced compared with normal, its expression was much lower in the HbSS erythrocytes (data not shown).

We are first using unsupervised analysis to analyze all the samples to see how they will be grouped together. It is possible that all HbSS samples are grouped either with the HbSA samples or the PNH/hemolytic anemia patients based on their gene expression. We are also defining gene signatures of HbS (based on HbSA vs. normal) and hemolytic anemia (PNH/hemolytic anemia vs. normal). We are examining their relative contribution at two levels. First, we are determining what genes are induced/repressed in these two conditions and are examining their expression in the HbSS erythrocytes. Second, we are determining the quantitative scores reflecting these two processes in the normal vs. HbSS erythrocytes, similar to the hypoxia response (Chi et al. (2006) PLoS Med. 3: e47), wound score (Chang et al. (2004) PLoS Biol. 2: E7) and reticulocyte score we have defined elsewhere herein. We are also performing array analysis based only on non-reticulocyte miRNA after the removal of the 83 reticulocyte-specific miRNA to rule out the potential complicating factors caused by contaminating reticulocytes.

Confirmation of the Expression Pattern of Anemia-Specific MiRNAs with TaqMan Assays

We are using real-time assays to independently confirm the differential expression of miRNAs discovered with microarrays. We are also validating the expression of these miRNAs in an independent set of samples to test the possibility of using erythrocyte miRNA for diagnostic and predictive purposes.

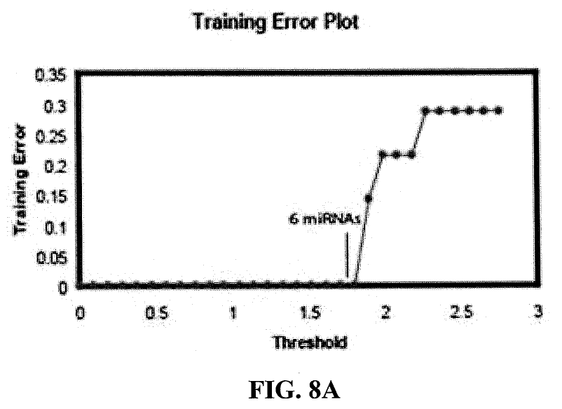

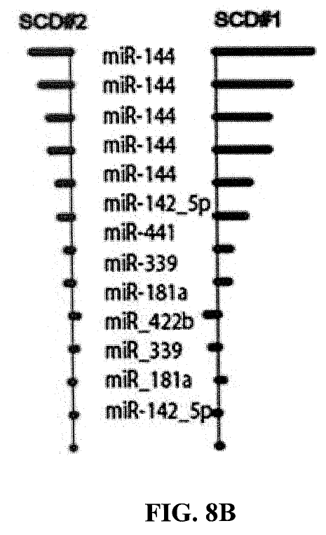

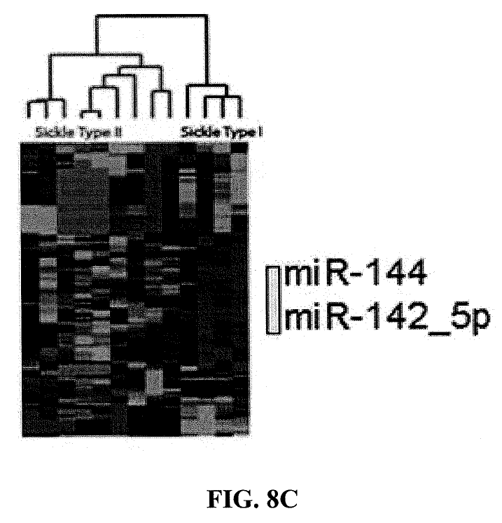

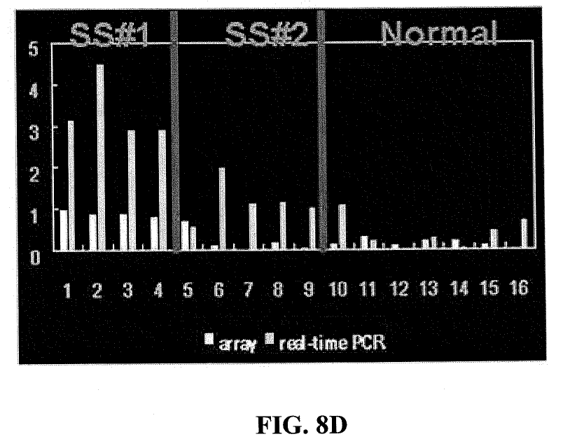

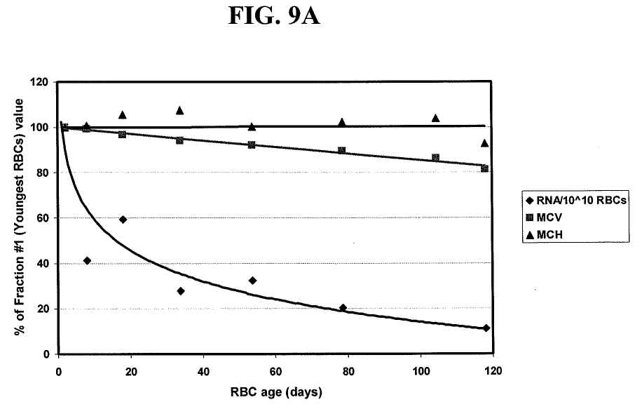

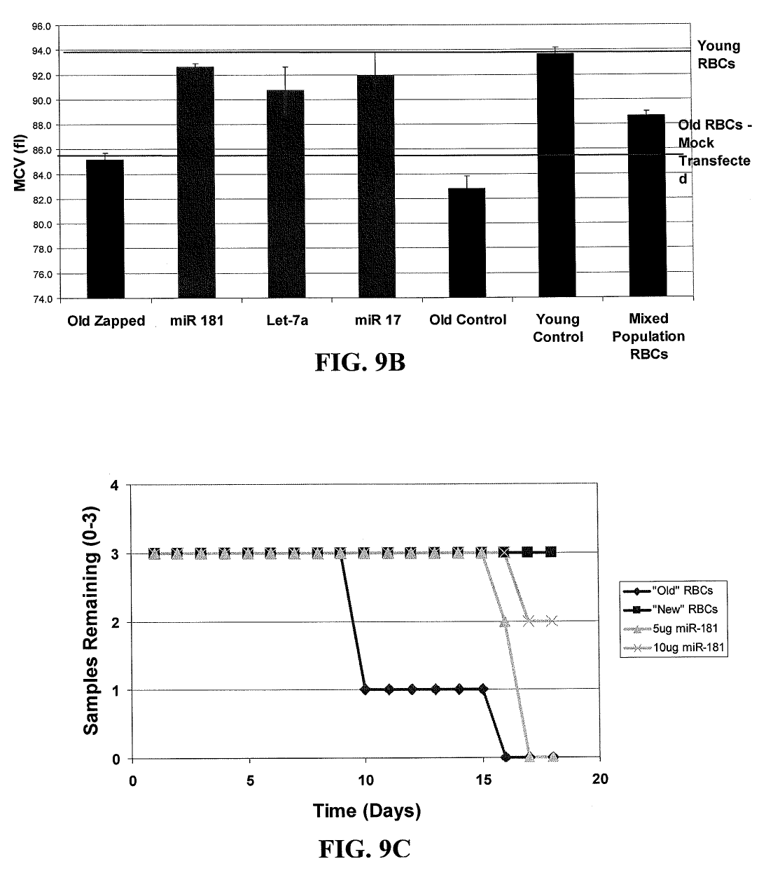

Although microarrays are wonderful tools for discovery, various issues have precluded their general applicability in the clinical setting. It is important to translate the discoveries from genomic studies into diagnostic tests using well-established methods. One way to achieve this transition is to first use microarrays to identify small sets of genes associated with investigated phenotypes. This will then allow the measurement of the expression level of these genes by the use of quantitative RT-PCR TaqMan assay, which is reproducible, reliable and compliant with current regulatory policy (Lossos et al. (2004) N. Engl. J. Med. 350: 1828-183). But, one challenge is how to select a limited number of genes from microarray studies while retaining the distinguishing power contained in the analysis of all genes. PAM (Prediction Analysis of Microarray) is the appropriate analytic tool for this purpose (Tibshirani et al. (2002) Proc. Natl. Acad. Sci. USA 99: 6567-6572). PAM is a class prediction tool based on the shrunken centroids of gene expression to identify subsets of genes that best characterize each assigned class. Briefly, the method computes a standardized centroid for each class. This is the average gene expression for each gene in each class divided by the within-class standard deviation for that gene. Nearest centroid classification takes the gene expression profile of a new sample, and compares it to each of these class centroids. The class whose centroid that it is closest to, in squared distance, is the predicted class for that new sample. After shrinking the centroids, the new sample is classified by the usual nearest centroid rule, but using the shrunken class centroids. Any genes that are shrunk to zero for all classes are eliminated from the prediction rule. PAM can thus select a number of genes with the ability to characterize each assigned class. Importantly, we can apply different shrinkage criteria to obtain different numbers of predictor genes and evaluate their performance in terms of error rate of the prediction (FIG. 8A). This will allow us to select an optimal number of genes in our predictor model to be used in TaqMan assays. Recently ABI has also developed TaqMan assays for miRNAs which are sensitive, accurate and reproducible (Chen et al. (2005) Nucleic Acids Res. 33: e179) (details below). When we applied PAM to analyze the best miRNA predicting the two HbSS subtypes, we found that we only need 6 miRNA probes to achieve 100% accuracy of class prediction. Five top probes are for miR-144 (FIG. 8B, C). Real-time RT-PCR assay for miR-144 shows that it is highest expressed in type I SCD, expressed at lowest level in normal erythrocytes with intermediate expression in type I SCD (FIG. 8D). One of the predicted target mRNAs for miR-144 is ETS-1, a transcription factor inhibiting erythropoeisis (Lulli et al. (2006) Cell Death Differ. 13: 1064-1074; Marziali et al. (2002) Blood Cells Mol. Dis. 29: 553-561; and Marziali et al. (2002) Oncogene 21: 7933-7944). The high level of miR-144 may be associated with low ETS-1 and increased erythropoeisis.

We are using PAM in two sets of samples. First, we are identifying miRNA whose expression can predict the genotypes normal vs. HbSA vs. HbSS. Since the HbS genotype is easily established, this analysis provides the proof-of-principle experiments to test whether miRNA expression, assayed with RT-PCR, can indeed reflect these known genotypes. We are then identifying the clinically relevant HbSS subtypes, such as those we have discovered in the previous steps (FIG. 3A). The current candidates are miR-144 and miR-142-5p. The methods for the stem-loop RT-PCR assays for one and multiple miRNAs have been described (Chen et al. (2005) Nucleic Acids Res. 33: e179; and Lao et al. (2006) Biochem. Biophys. Res. Commun. 343: 85-89). This assay only detects the mature form of miRNA, requires as little as 100 ng of RNA, and has the dynamic range of 10⁷ (Chen et al. (2005) Nucleic Acids Res. 33: e179). Briefly, step 1 is a reverse transcription reaction which reverse transcribes targeted miRNAs into cDNAs in a single reaction using miRNA-specific stem-loop RT primers; step 2 is a multiplexed PCR reaction with miRNA-specific forward primers and a universal reverse primer that amplifies the cDNA products to provide enough samples for step 3. Linear amplification is achieved with 14 PCR cycles. Step 3 is done as simultaneous, individual and multiplex TaqMan real-time PCR reactions to monitor the abundance of each miRNA after the RT-PCRs in an AB 7900 HT Sequence Detection System in a 384-well plate format. The abundance of the miRNA will be reflected by the number of PCR cycles required to reach detection threshold (Ct) when normalized against the endogenous control genes (−ΔCt). We are using the U6 snoRNA and miR-152 as an endogenous control for all erythrocyte samples given their constant and abundant expression (FIG. 1D).

### The Investigation of the Functional Role of MiRNAs in Determining Erythrocyte Phenotypes in SCD

The identification of particular miRNAs associated with SCD phenotypes leads to testable biological hypotheses for the relevant pathophysiological mechanisms. These molecular markers may also identify HbSS individuals with varying degrees of pathogenic processes and who are most likely to benefit from treatments targeting such processes. This strategy of patient stratification is the goal of “personalized medicine” and has been instrumental in the success of trastuzumab as a targeted therapy for patients with HER-2 over-expressing breast cancers (Slamon et al. (2001) N. Engl. J. Med. 344: 783-792). Our approach may offer similar benefit for SCD patients.

There are two possible mechanisms by which high miR-144/miR-142-5p might lead to high reticulocytosis/severe anemia.

One of the best-predicted targets for miR-144 and miR-142-5p, the two top miRNAs that distinguish subtype I from subtype II SCD, is NR-F2 (NFE2L2). NR-F2 is a transcription activator important for the coordinated up-regulation of genes defending oxidative stress (Lee et al. (2005) Faseb J. 19: 1061-1066; and Kensler et al. (2007) Annu. Rev. Pharmacol. Toxicol. 47: 89-116). In mice with disrupted NR-F2, a severe hemolytic anemia phenotype is noted due to increased sensitivity to oxidative stress (H2O2) (Lee et al. (2004) Proc. Natl. Acad. Sci. USA 101: 9751-9756). Oxidative stress is an important pathological factor in SCD pathogenesis since it changes the membrane stiffness and microrheological properties of erythrocyte, among other effects (Steinberg and Brugnara (2003) Annu. Rev. Med. 54: 89-112; and Hebbel, Leung and Mohandas (1990) Blood 76: 1015-1020). The intense oxidant nature of SCD is also reflected in the global transcriptional analysis of circulating leukocytes (Jison et al. (2004) Blood 104: 270-280). Altered free radical levels are also associated with different sensitivities to hemolysis and osmotic stress of sickle erythrocytes (Tatum and Chow (1996) Free Radic. Res. 25: 133-139). In addition to a higher level of oxidative stresses, SCD cells are also known to have lower glutathione (GSH) content and many NR-F2-regulated anti-oxidative proteins and thus more susceptibility to oxidative stresses (Tatum and Chow (1996) Free Radic. Res. 25: 133-139; Chiu and Lubin (1979) J. Lab. Clin. Med. 94: 542-548; Schacter et al. (1988) Faseb J. 2: 237-243; Hebbel et al. (1982) J. Clin. Invest. 70: 1253-1259; and Amer et al. (2006) Br. J. Haematol. 132: 108-113). But the mechanism underlying this susceptibility is unknown. Exposure to antioxidants, such as N-acetyl-cysteine, vitamin C and vitamin E, alleviates SCD symptoms by decreasing oxidative stress and reducing oxidative damage (Amer et al. (2006) Br. J. Haematol. 132: 108-113; and Steinberg and Brugnara (2003) Annu. Rev. Med. 54: 89-112). Thus, one mechanism by which high levels of miR-144/miR-142-5p lead to severe anemia is through the targeting of the NR-F2 transcription factor, leading to an enhanced susceptibility to oxidative stress.

The second mechanism by which miR-144/miR-142-5p affects sickle cell phenotypes is through decreased cell volume. When compared with other erythrocyte miRNAs or control DNA, both miR-144/miR-142-5p led to a significantly decreased MCV and increased MCHC when over-expressed in both normal and HbSS transfected erythrocytes (data not shown). This observation has significant pathophysiological relevance, since the rate of HbS polymerization and red cell sickling depends highly on the MCV and HbS concentration (Joiner (1993) Am. J. Physiol. 264: C251-270). The decrease in MCV caused by dehydration with increased Gardos channel activity increases HbS polymerization and erythrocyte sickling (Lew and Bookchin (2005) Physiol. Rev. 85: 179-200). Gardos channel inhibitors can thus improve the hydration status of sickle cells, increase MCV, and inhibit hemolysis (Stocker et al. (2003) Blood 101: 2412-2418). The decreased MCV induced by high levels of miR-144/miR-142-5p in HbSS subtype I may lead to erythrocyte sickling and hence more hemolysis. We are determining whether decreased MCV/increased MCHC induced by miR-144/miR-42-5p leads to increased sickling, decreased membrane deformability and changes in viscosity, characteristics leading to increased erythrocyte sickling and more hemolysis. To test these biophysical and rheological effects caused by miR-144/miR-142-5p, we have established a cooperation with Dr. Herbert Meiselman in the CSCC in the Medical School of the University of Southern California, who is an expert in measuring the biophysical and rheological properties of sickle cells (Lee et al. (2007) Biorheology 44: 29-35; Jing et al. (2005) Cell 120: 623-634; Alexy et al. (2006) Transfusion 46: 912-918; Leaman et al. (2005) Cell 121: 1097-1108; Malik et al. (1998) Blood 91: 2664-2671; Walters et al. (1996) N. Engl. J. Med. 335: 369-376; and Hofstra et al. (1996) Blood 87: 4440-4447). This effect can last for more than 4 days at 4° C., making it possible to ship the transfected erythrocytes overnight to California for measurement. We are also testing whether miRNA inhibition can alter the MCV, MCHC and cell sickling. This sequence-specific inhibition is usually based on blocking base-pairing essential for miRNA function and may not be directly applicable here. However, an aptamer binding to coagulation factor IX can be reversibly blocked by its complementary sequence as a result of the change in conformation caused by the formation of double-strand pairing (Rusconi et al. (2002) Nature 419: 90-94). This provides the rationale to test whether miRNA over-expression or inhibition can affect the MCV, MCHC, and, in turn, the tendency of cells to undergo sickling under hypoxia and other stresses.

### Example 4

### The Determination of the Role of Erythrocyte miRNA in the Different In Vitro Susceptibility to Plasmodium falciparum Seen in SCD

The malaria susceptibility of erythrocytes is altered in several hemoglobinopathies and this relative resistance to malaria is thought to be responsible for the positive selection of these mutant alleles (Nagel et al. (1989) Blood 74: 1213-1221). The present invention recognizes that the different miRNA expression pattern in these erythrocytes contributes to their resistance to malaria. This is consistent with our preliminary data and one published study (Rathjen et al. (2006) FEBS Lett. 580: 5185-5188), indicating that small subsets of erythrocyte miRNAs are found in P. falciparum and can impact the parasite replication and growth (FIGS. 5 and 6). Here, we show that the differing miRNA expression in normal, HbSA and HbSS erythrocytes contributes to the varying in vitro susceptibility to P. falciparum. We are identifying miRNAs associated with differing susceptibility to P. falciparum. We are then testing the functions of these miRNAs by examining how overexpression and blockage of these miRNAs in erythrocytes will affect the growth and survival of P. falciparum.

The Determination of the In Vitro Susceptibility to P. falciparum of Normal, HbSA and HbSS Erythrocytes

We have shown that much lower parasitemia was achieved when P. falciparum was propagated within the HbSS erythrocytes (FIG. 6F), consistent with several published studies (Friedman, M. J. (1978) Proc. Natl. Acad. Sci. USA 75: 1994-1997; and Pasvol et al. (1978) Nature 274: 701-703). Since the HbSA is the main population selected by malaria (Aidoo et al. (2002) Lancet 359: 1311-1312), we are expanding our studies to include more normal, HbSS and HbSA samples and are assaying their in vitro susceptibility simultaneously in parallel. Furthermore, we are also obtaining the global miRNA expression with miRNA arrays on the same erythrocytes. The availability of phenotypes (P. falciparum susceptibility) and genotypes (HbS status and miRNA gene expression) in the same set of samples allow us to derive important phenotype-genotype correlation. We are hosting a blood drive event in the Duke CSCC and obtain erythrocyte samples from 10 HbSS, 10 HbSA and 10 normal healthy African American individuals. We are then diluting the same number of synchronized parasites into an equal number of erythrocytes (normalized by hematocrit and cell count). We are assessing the degree of parasitemia with Giemsa stain and FACS after the parasites in the permeabilized infected erythrocyte are stained with YoYo-1 at 2, 4, 6, and 8 days (Barkan et al. (2000) Int. J. Parasitol. 30: 649-653). Alternatively, we are assessing the miRNAs' influence on in vitro Plasmodium DNA replication by assessing [³H]hypoxanthine incorporation (Chulay et al. (1983) Exp. Parasitol. 55: 138-146).

We expect that the normal erythrocytes will be most susceptible to malaria and the HbSS erythrocyte will be most resistant to malaria growth with the correspondingly highest and lowest level of parasitemia (FIG. 6F). The HbSA erythrocyte is likely to be intermediate in its susceptibility. But, there are also potential significant variations in each of the three groups. In the subsequent analysis of finding miRNA correlated with susceptibility, we are analyzing the erythrocytes based on the known three groups of HbSS, HbSA and normal status as well as purely based on their determined in vitro susceptibility. We are then using SAM and rank-sum test to analyze miRNA expression data to identify miRNAs associated with normal vs. HbSA vs. HbSS as well as their high vs. low measured in vitro susceptibility.

The Identification of the Erythrocyte MiRNAs which are Translocated into P. falciparum During Infection within the Normal, HbSA and HbSS Erythrocytes

We have identified several miRNAs which are translocated into parasites during their growth in normal erythrocytes (FIG. 6). Since the miRNA composition and other cellular properties are different in the HbSA and HbSS erythrocytes, quantitative and/or qualitative differences in the translocated miRNA are analyzed.

We are expanding the synchronized culture of P. falciparum in 3 samples of normal, HbSA and HbSS erythrocytes used for determining the susceptibility in SA2a. We are then isolating RNAs from the uninfected erythrocytes cultured in parallel and erythrocyte-free parasites at 12, 24, 36 and 48 hour. Given the limited number of cells and low amount of RNA we obtain, we are using the multiplex ABI real-time assays to determine the composition and relative abundance of miRNAs. We are using the expression level of P. falciparum 18S RNA to serve as an internal control to calculate −ΔCt as a reflection of the miRNA abundance. We are then ranking all miRNAs found in the parasites based on their −ΔCt value to prioritize their likelihood of parasite translocation. We are also focusing on the quantitative comparison between the amount of miR-223 and miR-451 found in the HbSA and HbSS erythrocytes compared with normal erythrocytes as well as identifying the miRNAs which are found only when grown in HbSA and HbSS erythrocytes. We are further validating the actual miRNA translocation with immunohistochemistry assays.

The Identification of the MiRNAs Whose Expression is Likely to Associate with Different Host Susceptibility to P. falciparum

We are identifying several (4-6) miRNAs which are likely to play a role in erythrocyte-P. falciparum interaction for further functional characterization. We are integrating several lines of information to identify candidate miRNAs: 1) the presence in the parasites grown in either normal, HbSA or HbSS erythrocytes; 2) the correlation with an increase or decrease in host susceptibility; and 3) the altered expression in the HbSA and/or HbSS erythrocytes. We are using supervised analysis SAM to identify miRNAs with specific expression in normal, HbSA and HbSS erythrocytes or associated with in vitro susceptibility. This information leads to a gene list containing all the translocated miRNAs and their relative expression levels in normal vs. HbSA vs. HbSS erythrocytes are determined. For example, we have found that miR-451 is highly expressed in all and miR-223 is overexpressed in approximately 30% of HbSS erythrocytes (FIG. 6). Although both miR-223 and miR-451 seem to be inhibitory for parasite growth, it is possible that other miRNAs may suppress or enhance the parasite growth. Therefore, this work is identifying miRNAs both positively and negatively correlated with in vitro susceptibility.

The Determination of the Functional Roles of Erythrocyte MiRNAs in Regulating the Growth and Survival of P. falciparum in Erythrocytes.

We are testing the functional effects of individual miRNAs on the growth and replication of P. falciparum. Several recent reports have shown the use of RNA interference (RNAi) in gene silencing in Plasmodium parasites (Malhotra et al. (2002) Mol. Microbiol. 45: 1245-1254; and Mohmmed et al. (2003) Biochem. Biophys. Res. Commun. 309: 506-511), suggesting the existence of RNAi machinery and the probable functional role of translocated miRNAs in Plasmodium.

Given the phenotypic alterations of P. falciparum caused by increased levels of erythrocytic miR-451, we decided to set up a P. falciparum reporter system to directly assess the effect of the translocated microRNAs. To achieve this purpose, we modified a pHLH1 luciferase reporter plasmid constructed for determining gene regulation in P. falciparum. This modified pHLH1 construct contains an ampicillin selectable marker, as well as histidine rich protein three (hrp3) 5′ and 3′ untranslated regions (UTRs) flanking the Firefly luciferase (fluc) gene (Wu et al. (1995) Proc. Natl. Acad. Sci. USA 92: 973-977). With the hypothesis that these translocated microRNA act in a similar fashion as either microRNA or anti-sense RNA in mammalian cells, we placed two repeat copies of the miR-451 target sequences in both orientations (sense (complementary to miR451) and anti-sense) into the HindIII restriction site between the luciferase ORF and the hrp3 3′ UTR behind the luciferase sequence (Gottwein et al. (2007) Nature 450: 1096-1099; AND Wu et al. (1995) Proc. Natl. Acad. Sci. USA 92: 973-977). These reporter plasmids were electroporated into erythrocytes and subsequently infected with P. falciparum for 48 hrs according to previously published protocols (Crabb et al. (2004) Methods Mol. Biol. 270: 263-276) before the parasites were isolated by saponin treatment and lysed for the reporter assay. The specificity of this microRNA-mediated biological effect was then determined by the observed luciferase activity after the transfection of either control reporter without any additional inserts or reporter constructs with inserts in the 3′UTR in a “sense” vs. “anti-sense” orientation (data not shown). Results from the luciferase assay showed that compared to the control plasmids containing none or anti-sense strands, the construct with the sense strand demonstrated an 80% reduction in reporter activity (data not shown). This difference was presumably caused by the translocated miR-451 and was reproducible in two independent experiments. We are improving the assays with more testing and the inclusion of second luciferase (Renilla) as an internal control in a dual luciferase system.

The establishment of such a reporter system allows us to address the level of gene regulation mediated by translocated microRNAs. When we obtain potential candidate mRNA targets from our experimental and bioinformatics efforts, we will be able to test their potential as direct regulatory targets for miR-451 and other microRNAs. This reporter assay will allow us to further enhance our understanding of the molecular mechanisms by which translocated erythrocyte microRNAs impact the gene regulation of Plasmodium falciparum and contribute to the scientific success of the proposed experiments.

We are first overexpressing the investigated miRNAs in normal erythrocytes and assaying the impact on the growth and differentiation of synchronized P. falciparum. We are using three different assays 1) Giemsa stain to evaluate the progression to ring, trophozoite, and schizont stage of infection as well as the percentage of infected erythrocytes; 2) FACS analysis of YoYo-1 staining to assay the level of parasitemia; and 3) [³H]hypoxanthine incorporation to assess the miRNAs' influence on in vitro Plasmodium DNA replication (Chulay et al. (1983) Exp. Parasitol. 55: 138-146). We are also assaying the effect of the blockage of particular miRNAs in normal erythrocytes using one of two frequently used assays, based either on LNA (Fazi et al. (2005) Cell 123: 819-831) or 2′-O-methyl oligonucleotides (Hutvagner et al. (2004) PLoS Biol. 2: E98) complementary to miRNA sequences to block the miRNA functions.

Furthermore, we are normalizing the miRNA expression in HbSA and HbSS erythrocytes and assaying the change in the malaria susceptibility afterwards. For the translocated miRNAs whose expression is upregulated in sickle cells (which is the case for both miR-451 & miR-223), the malaria susceptibility is compared after selected miRNAs have either been inhibited by transfecting 2′-O-methyl oligonucleotides complementary to its sequence (Hutvagner er al (2004) PLoS Biol. 2: E98) or control miRNA or GFP (Meister et al. (2004) RNA 10: 544-550). Alternatively, if the miRNAs are poorly expressed in the sickle cell erythrocytes, we test the effect of miRNA overexpression on parasite growth. We have observed a reproducible increase in parasitemia when we block the miR-451 in three HbSS erythrocytes (FIG. 6F), suggesting that high miR-451 expression does contribute to the malaria resistance of HbSS erythrocytes. This result is very important since it shows that blocking the endogenous miRNAs can lead to enhanced infection and increased susceptibility. We are extending this analysis to other miRNAs with potential roles in affecting the intraerythrocytic biology of P. falciparum to determine their functional role in vivo. These series of experiments allow us to formally determine the relative contribution of erythrocyte miRNAs to the varying susceptibility of different erythrocytes.

### Example 5

### “Rejuvenation” of Old Erythrocytes with microRNA Replacement

After reticulocytes undergo terminal differentiation to become mature erythrocytes, these “young” erythrocytes have a life-span of 120 days in the peripheral circulation until they become “old” erythrocytes and are removed from the peripheral circulation by the immune system. The young and old red cells are very different in many biological properties. The young cells are larger (as measured in Mean Corpuscular Volume MCV) and of lower density. Young cells also survive for much longer both in vivo and in vitro. It is generally assumed that a similar aging process is also responsible for the limited preservation of blood products during in vitro storage in blood banks. Although these differences are well established, the molecular mechanisms underlying these differences are unknown.

The different cell density of young vs. old erythrocytes allows them to be separated by a density gradient. In contrast to most measured physiological parameters which are largely similar between young vs. old erythrocytes, the RNA content per erythrocytes differs by 10 fold (FIG. 10A). This difference is expected since there is no novel RNA synthesis and the lower RNA content reflects the gradual RNA decay in the absence of novel RNA synthesis. This finding caused us to examine whether replenishing the RNA content of old erythrocytes would rejuvenate them to assume a younger phenotype. Indeed, transfection of several miRNAs (miR-181a, Let-7a-1, and miR-17) into old erythrocytes leads to an increase in MCV of older red blood cells to similar MCV observed in the youngest red blood cells (FIG. 10B). To test whether microRNA replacement will lead to longer survival of old erythrocytes, we transfected miR-181a into old erythrocytes and measured their in vitro survival compared with mock transfected erythrocytes. We have found that over-expression of miR-181a in old erythrocytes can lead to an increase in their survival during in vitro culture (FIG. 10C). Collectively, we have found that microRNA replacement in the old erythrocyte can “rejuvenate” them to assume a younger phenotype—with higher cell volume and longer survival in vitro.

Table 2 provides the sequences of many of the erythrocyte microRNAs referred to throughout the application.

TABLE 2
Erythrocyte microRNA sequences

| | | |
|---|---|---|
| MicroRNA | Sequence | SEQ ID NO: |

| | | |
|---|---|---|
| hsa-miR-181a | 5′-AACAUUCAACGCUGUCGGUGAGU-3′ | 7 |
| hsa-Let-7a-1 | 5′-UGAGGUAGUAGGUUGUAUAGUU-3′ | 8 |
| hsa-miR-17 | 5′-CAAAGUGCUUACAGUGCAGGUAG-3′ | 9 |
| hsa-miR-320 | 5′-AAAAGCUGGGUUGAGAGGGCGA-3′ | 10 |
| hsa-miR-144 | 5′-UACAGUAUAGAUGAUGUACU-3′ | 11 |
| hsa-miR-142-5-p | 5′-CAUAAAGUAGAAAGCACUACU-3′ | 12 |
| hsa-miR-451 | 5′-AAACCGUUACCAUUACUGAGUU-3′ | 13 |
| hsa-miR-223 | 5′-UGUCAGUUUGUCAAAUACCCCA-3′ | 14 |
| hsa-miR-23b | 5′-AUCACAUUGCCAGGGAUUACC-3′ | 27 |
| hsa-miR-221 | 5′-AGCUACAUUGUCUGCUGGGUUUC-3′ | 28 |

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for determining the susceptibility to an erythrocyte disease in a subject or for determining the severity of an erythrocyte disease in a subject, said method comprising obtaining a sample of erythrocytes from said subject, determining the composition of miRNA present in said erythrocytes wherein the composition of miRNA is predictive of susceptibility to or severity of an erythrocyte disease.

2. The method of claim 1 wherein said erythrocyte disease is anemia or malaria.

3. The method of claim 2, wherein said erythrocyte disease is anemia, wherein said composition of miRNA comprises at least one of miR-144 and miR-142-5p, and wherein an increase in the level of at least one of miR-144 and miR-142-5p compared to a control indicates an enhanced susceptibility of said subject to said erythrocyte disease or a more severe erythrocyte disease relative to said control.

4. The method of claim 3, wherein said subject has a sickle-cell disease.

5. The method of claim 2, wherein said erythrocyte disease is malaria, wherein said composition of miRNA comprises a miRNA capable of translocating into a malaria parasite and inhibiting the growth or survival of said malaria parasite, and wherein an increase in the level of said miRNA compared to a control indicates an enhanced susceptibility of said subject to said erythrocyte disease or a more severe erythrocyte disease relative to said control.

6. The method of claim 5, wherein said miRNA capable of translocating into a malaria parasite comprises at least one of miR-451 and miR-223.

7. The method of claim 5, wherein said subject has a sickle-cell disease or sickle-cell trait.

8. The method of claim 1, wherein said erythrocyte disease is a sickle-cell disease.

9. The method of claim 8, wherein said composition of miRNA comprises a reticulocyte-specific miRNA, and wherein an increase in the level of said reticulocyte-specific miRNA compared to a control indicates a more severe sickle-cell disease relative to said control.

10. The method of claim 8, wherein said composition of miRNA comprises at least one of miR-144 and miR-142-5p, and wherein an increase in the level of at least one of miR-144 and miR-142-5p compared to a control indicates a more severe sickle-cell disease relative to said control.

11. The method of claim 8, wherein said composition of miRNA comprises at least one of miR-320, miR-23b, and miR-221, and wherein a decrease in the level of at least one of miR-320, miR-23b, and miR-221 indicates a more severe sickle-cell disease relative to said control.

12. A method for treating a subject with anemia, said method comprising administering to said subject a composition selected from the group consisting of:
a) a polynucleotide comprising or encoding a miRNA or a precursor thereof, wherein said miRNA has a nucleotide sequence having at least 90% sequence identity to SEQ ID NO: 10, and wherein said polynucleotide when expressed or introduced into a red blood cell enhances the survival of said red blood cell;
b) a polynucleotide comprising or encoding a miRNA or a precursor thereof, wherein said miRNA has a nucleotide sequence having at least 90% sequence identity to SEQ ID NO: 7, 8 or 9, and wherein said polynucleotide when expressed or introduced into an erythrocyte increases the life-span of said erythrocyte; and
c) a compound that inhibits the activity of at least one of miR-144 and miR-142-5p.

13. The method of claim 12, wherein said subject has a sickle cell disease.

14. The method of claim 12, wherein said compound comprises a polynucleotide comprising or encoding a nucleotide sequence that is complementary to a miRNA, wherein said miRNA has a nucleotide sequence having at least 90% sequence identity to SEQ ID NO: 11 or 12.

15. A method for treating a subject with malaria or for reducing the susceptibility of a subject to malaria, said method comprising administering to said subject a polynucleotide comprising or encoding a miRNA or a precursor thereof, wherein said miRNA has a nucleotide sequence having at least 90% sequence identity to SEQ ID NO: 13 or 14, and wherein said miRNA has the ability to be translocated into a malaria parasite infecting a red blood cell and to inhibit the growth or survival of said malaria parasite.

16. A method for the identification of an erythrocyte miRNA that is associated with an erythrocyte disease, said method comprising obtaining erythrocytes from a population of subjects, wherein said population comprises subjects having said erythrocyte disease, determining the composition of miRNA present in said erythrocytes from each subject within the population, and performing an analysis of the miRNA composition from each subject within the population to identify an erythrocyte miRNA that is predictive of the susceptibility to or severity of an erythrocyte disease, is indicative of the presence of the erythrocyte disease, or that distinguishes subtypes of the erythrocyte disease.


SEQUENCE LISTING (28 sequences)

SEQ ID NO: 1 - DNA, 26 bp, Homo sapiens
tttaggcagcagcttttaatacaggg

SEQ ID NO: 2 - DNA, 26 bp, Pan troglodytes
tttaggcagcagcttttaatacaggg

SEQ ID NO: 3 - DNA, 25 bp, Mus musculus
ttaggcagcagcttttagtgcgggg

SEQ ID NO: 4 - DNA, 26 bp, Rattus norvegicus
tttaggcagcagcttttagtgcaggg

SEQ ID NO: 5 - DNA, 26 bp, Canis lupus
tttaggcagcagcttttcatacgggg

SEQ ID NO: 6 - RNA, 23 nt, Homo sapiens
aagcgggagaguugggucgaaaa

SEQ ID NO: 7 - RNA, 23 nt, Homo sapiens
aacauucaacgcugucggugagu

SEQ ID NO: 8 - RNA, 22 nt, Homo sapiens
ugagguaguagguuguauaguu

SEQ ID NO: 9 - RNA, 23 nt, Homo sapiens
caaagugcuuacagugcagguag

SEQ ID NO: 10 - RNA, 22 nt, Homo sapiens
aaaagcuggguugagagggcga

SEQ ID NO: 11 - RNA, 20 nt, Homo sapiens
uacaguauagaugauguacu

SEQ ID NO: 12 - RNA, 21 nt, Homo sapiens
cauaaaguagaaagcacuacu

SEQ ID NO: 13 - RNA, 22 nt, Homo sapiens
aaaccguuaccauuacugaguu

SEQ ID NO: 14 - RNA, 22 nt, Homo sapiens
ugucaguuugucaaauacccca

SEQ ID NO: 15 - DNA, 20 bp, Artificial Sequence
  oligonucleotide primer
gcaacctcaaacagacacca

SEQ ID NO: 16 - DNA, 20 bp, Artificial Sequence
  oligonucleotide primer
agctcactcagtgtggcaaa

SEQ ID NO: 17 - DNA, 22 bp, Artificial Sequence
  oligonucleotide primer
acgtaatggaagtgctgcaatg

SEQ ID NO: 18 - DNA, 29 bp, Artificial Sequence
  oligonucleotide primer
gcatactattatctgatgtcatggagaca

SEQ ID NO: 19 - DNA, 20 bp, Artificial Sequence
  oligonucleotide primer
atgtgatacccatagcttcc

SEQ ID NO: 20 - DNA, 20 bp, Artificial Sequence
  oligonucleotide primer
ggcttagatctcatttggag

SEQ ID NO: 21 - DNA, 28 bp, Artificial Sequence
  oligonucleotide primer
ctagatgtctttaggcaggatcctttaa

SEQ ID NO: 22 - DNA, 28 bp, Artificial Sequence
  oligonucleotide primer
ctagatgtctttaggcagcagcttttaa

SEQ ID NO: 23 - DNA, 28 bp, Artificial Sequence
  oligonucleotide primer
ccgaattccaggaaccagacagggacgc

SEQ ID NO: 24 - DNA, 28 bp, Artificial Sequence
  oligonucleotide primer
ccctcgagccgactcttaagtccaggtc

SEQ ID NO: 25 - DNA, 29 bp, Artificial Sequence
  oligonucleotide primer
ccgaattcacagtgcttttcaagccatgc

SEQ ID NO: 26 - DNA, 29 bp, Artificial Sequence
  oligonucleotide primer
ccctcgagatcctcctgccttggcctctg

SEQ ID NO: 27 - RNA, 21 nt, Homo sapiens
aucacauugccagggauuacc

SEQ ID NO: 28 - RNA, 23 nt, Homo sapiens
agcuacauugucugcuggguuuc